(19) United States
(12) Patent Application Publication
(10) Pub. No.: US 2025/0262173 A1
(43) Pub. Date: Aug. 21, 2025

(54) INTRANASAL EPINEPHRINE FORMULATIONS AND METHODS OF USE

(71) Applicant: ARS Pharmaceuticals Operations, Inc., San Diego, CA (US)

(72) Inventor: Richard LOWENTHAL, San Diego, CA (US)

(21) Appl. No.: 19/056,061

(22) Filed: Feb. 18, 2025

Related U.S. Application Data
(60) Provisional application No. 63/555,207, filed on Feb. 19, 2024.

(51) Int. Cl. A61K 31/137; A61K 9/00; A61K 9/08; A61K 47/02; A61K 47/10; A61K 47/12; A61K 47/18; A61K 47/22; A61K 47/26; A61P 11/02

(52) U.S. Cl. CPC A61K 31/137; A61K 9/0043; A61K 9/08; A61K 47/02; A61K 47/10; A61K 47/12; A61K 47/183; A61K 47/186; A61K 47/22; A61K 47/26; A61P 11/02

(57) ABSTRACT

Drug products adapted for nasal delivery comprising formulations with epinephrine and devices comprising such formulations are provided. Methods of treating anaphylaxis with epinephrine products are also provided.

### CROSS-REFERENCE TO RELATED APPLICATIONS

This application claims the benefit of U.S. Provisional Patent Application No. 63/555,207, filed Feb. 19, 2024, which is incorporated herein by reference in its entirety.

### FIELD OF THE INVENTION

Described herein are intranasal (IN) epinephrine formulations and methods of using such formulations in the treatment of conditions or diseases.

### BACKGROUND OF THE INVENTION

Anaphylaxis is a medical emergency that may require resuscitation measures such as airway management, supplemental oxygen, large volumes of intravenous fluids, and close monitoring. Administration of epinephrine is the treatment of choice. A need exists for needle-free and non-invasive methods of dosing epinephrine. Provided herein are methods, formulations, and devices for the treatment of anaphylaxis and other conditions.

### SUMMARY OF THE INVENTION

Disclosed herein are methods, pharmaceutical formulations of epinephrine and methods of use thereof in the treatment of conditions such as type-1 hypersensitivity reactions (systemic allergic reaction), asthma, and cardiac arrest.

Anaphylaxis is a severe, potentially life-threatening type-1 hypersensitivity reaction (systemic allergic reaction) that affects many body systems, with rapid onset typically averaging between about 5 to 30 minutes after exposure to an antigen and about 2 hours after oral exposure.

Anaphylaxis results from the release of inflammatory mediators and cytokines from mast cells and basophils, typically due to an immunologic reaction, but sometimes due to non-immunologic mechanisms. The most common areas of the body affected include: skin (80-90%), respiratory (70%), gastrointestinal (30-45%), heart and vasculature (10-45%), and central nervous system (10-15%) with usually two or more being involved in a single episode.

Anaphylaxis is a medical emergency that may require resuscitation measures such as airway management, supplemental oxygen, large volumes of intravenous fluids, and close monitoring.

Administration of epinephrine is the treatment of choice with antihistamines and steroids (for example, dexamethasone) often used as adjuncts. Due to concerns of biphasic anaphylaxis, a period of in-hospital observation for between 2 and 24 hours is often required for people once they have returned to normal.

Epinephrine (adrenaline, (R)-4-(1-Hydroxy-2-(methylamino)ethyl)benzene-1,2-diol) is the primary treatment for anaphylaxis with no absolute contraindication to its use. Currently epinephrine is administered as a solution given by injection, preferably into the mid anterolateral thigh as soon as anaphylaxis is suspected. The injection may be repeated every 5 to 15 minutes if there is insufficient response. A second dose is needed in 16-35% of episodes, but more than two doses are rarely required. The intramuscular route is preferred over subcutaneous administration because the latter may have delayed epinephrine absorption. However, while only minor adverse effects from epinephrine are reported (tremors, anxiety, headaches, and palpitations) there have been numerous reports of the highly variable exposures from injection products depending on the location of the injection (intramuscular or subcutaneous), and other factors such as body mass index (BMI).

There is a significant need in the medical community to develop products that will help improve the clinical management of anaphylaxis in an out-of-hospital setting. While epinephrine is effective when delivered by intramuscular injection, there is published evidence that the pharmacokinetics are highly variable depending on the site of the injection, whether intramuscular or subcutaneous. There have also been significant product quality problems with approved auto-injectors that utilize complex technologies, resulting in many recalls for these products by the FDA in the United States. Epinephrine auto-injectors, such as EpiPen®, are also cumbersome to carry, and require training and time to properly administer in a potentially life-threatening situation.

The need for alternative, needle-free and non-invasive methods for dosing epinephrine are well-documented, as many patients have a fear of injection and, as a result, are reluctant to use an auto-injector of any kind. Further, the auto-injectors are large and burdensome, so many patients in need do not have an epinephrine injector in their presence at all times. There is also a well-documented reluctance to self-administer a dose in public settings.

Thus, there is a need for improved or alternative methods of dosing epinephrine in an emergency situation, as well as improved or alternative formulations and devices. Desirable improvements include: individually and in combinations, convenience (intranasal versus intramuscular), more rapid administration, more reliable, more consistent dosing, needleless, more discrete to dose in public, and administrable by an untrained individual or non-professional.

Accordingly, provided herein are methods, formulations, and devices for the treatment of anaphylaxis and other conditions comprising administering an intranasal formulation of epinephrine using a small compact unit dose sprayer device.

In one aspect, described herein is a method of treating a type-1 hypersensitivity reaction in a human comprising intranasally administering to the human with the type-1 hypersensitivity reaction two or more doses of a pharmaceutical formulation of epinephrine, or a salt thereof; wherein each dose comprises between about 1.0 mg and about 2.0 mg of epinephrine, or a pharmaceutically acceptable salt thereof, and dodecyl maltoside; wherein the intranasal administration of two doses provides epinephrine pharmacokinetics in the human that is at least the same as the intramuscular injection of two 0.3 mg epinephrine doses in the anterolateral thigh; wherein the intranasal administration of each dose after the first dose provides dose proportional epinephrine pharmacokinetics; wherein each dose is intranasally administered in the same nostril; or wherein each dose is intranasally administered in opposite or alternating nostrils.

In some embodiments, each intranasally administered dose of the pharmaceutical formulation provides plasma epinephrine concentrations in the human that are efficacious for the elimination of, and/or for the prevention of, the further progression of, one, more than one, or all of the symptoms of the type-1 hypersensitivity reaction in the human; or the intranasal administration of the first dose of the pharmaceutical formulation to the human with the type-1 hypersensitivity reaction provides inadequate plasma epinephrine concentrations in the human that are efficacious for the elimination of, and/or for the prevention of, the further progression of, one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the elimination of, and/or for the prevention of the further progression of, one, more than one, or all of the symptoms of the type-1 hypersensitivity reaction in the human.

In some embodiments, the human with the type-1 hypersensitivity reaction has symptoms of rhinitis; wherein the symptoms of rhinitis comprise nasal edema, congestion, or both. In some embodiments, the type-1 hypersensitivity reaction comprises allergic asthma, allergic conjunctivitis, allergic rhinitis, anaphylaxis, angioedema, urticaria, eosinophilia, or combinations thereof. In some embodiments, the type-1 hypersensitivity reaction comprises allergic asthma, allergic conjunctivitis, allergic rhinitis, anaphylaxis, angioedema, urticaria, eosinophilia, drug allergy or a food allergy. In some embodiments, the type-1 hypersensitivity reaction comprises an antibiotic allergy. In some embodiments, the type-1 hypersensitivity reaction comprises anaphylaxis. In some embodiments, the type-1 hypersensitivity reaction comprises an allergic reaction to an insect sting or bite, venom, allergen immunotherapy, foods, drugs, diagnostic testing substances or other allergens, or combinations thereof. In some embodiments, the type-1 hypersensitivity reaction comprises idiopathic anaphylaxis or exercise-induced anaphylaxis. In some embodiments, the type-1 hypersensitivity reaction comprises anaphylaxis; and the symptoms of anaphylaxis are selected from the group consisting of hives, generalized itching, nasal congestion, wheezing, difficulty breathing, cough, cyanosis, lightheadedness, dizziness, confusion, slurred speech, rapid pulse, palpitations, nausea or vomiting, abdominal pain or cramping, skin redness or skin inflammation, nasal flaring, and intercostal retractions. In some embodiments, the type-1 hypersensitivity reaction comprises urticaria, and the symptoms of the type-1 hypersensitivity reaction comprise pruritis, flushing, or a burning sensation of the skin.

In some embodiments, the intranasal administration of two intranasal doses provides pharmacokinetics that are greater than intramuscular injection of two 0.3 mg doses in the anterolateral thigh. In some embodiments, the intranasal administration of the two intranasal doses provides plasma epinephrine concentrations with a Cₘₐₓ of at least 100 μg/mL, at least 200 μg/mL, or at least 300 μg/mL.

In some embodiments, the intranasal administration of the two intranasal doses provides plasma epinephrine concentrations with a time to maximum epinephrine plasma concentrations (Tmax) of less than 45 minutes, less than 35 minutes, less than 25 minutes, or less than 15 minutes.

In some embodiments, the pharmaceutical formulation is a nasal spray pharmaceutical formulation, and wherein the pharmaceutical formulation is a nasal spray pharmaceutical formulation comprising between about 1 mg/mL and about 40 mg/mL of epinephrine, or a salt thereof, in a volume between about 25 μL and about 250 μL per dose.

In some embodiments, each dose of the pharmaceutical formulation comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL, of epinephrine, or a salt thereof, in a volume between about 25 μL and about 250 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

In some embodiments, each dose of the pharmaceutical formulation comprises about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL of epinephrine, or a salt thereof, in a volume between about 50 L and about 200 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

In some embodiments, each dose of the pharmaceutical formulation comprises about 5 mg/mL, about 6 mg/mL, about 7 mg/mL, about 8 mg/mL, about 9 mg/mL, or about 10 mg/mL of epinephrine, or a salt thereof, in a volume of about 200 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

In some embodiments, each dose of the pharmaceutical formulation comprises about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof, in a volume of about 100 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

In some embodiments, each dose of the pharmaceutical formulation comprises about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL of epinephrine, or a salt thereof, in a volume of about 50 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

In some embodiments, each dose of the pharmaceutical formulation comprises about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg of epinephrine, or a salt thereof, in a volume between about 50 L and about 250 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

In some embodiments, the isotonicity agents are selected from the group consisting of dextrose, glycerin, mannitol, potassium chloride, and sodium chloride; wherein the stabilizing agent is ethylenediaminetetraacetic acid (EDTA) or disodium salt thereof; wherein the antioxidants are selected from the group consisting of alpha tocopherol, D-α-tocopherol polyethylene glycol 1000 succinate, ascorbic acid, isoascorbic acid, butylated hydroxyanisole, citric acid monohydrate, potassium metabisulfite, sodium bisulfite, sodium metabisulfite, and sodium sulfite; wherein the preservative is benzalkonium chloride; and wherein the pH adjustment agents are selected from the group consisting of adipic acid, ammonium chloride, citric acid, acetic acid, hydrochloric acid, lactic acid, phosphoric acid, propionic acid, sulfuric acid, tartaric acid, sodium hydroxide, sodium citrate, sodium bicarbonate, and sodium carbonate.

In some embodiments, the isotonicity agents are selected from the group consisting of dextrose, glycerin, mannitol, potassium chloride, and sodium chloride; wherein the stabilizing agent is ethylenediaminetetraacetic acid (EDTA) or disodium salt thereof; wherein the antioxidants are selected from the group consisting of alpha tocopherol, ascorbic acid, isoascorbic acid, potassium metabisulfite, sodium bisulfite, and sodium metabisulfite, sodium sulfite; wherein the preservative is benzalkonium chloride; and wherein the pH adjustment agents are selected from the group consisting of hydrochloric acid and sodium hydroxide.

In some embodiments, each intranasally delivered dose of the pharmaceutical formulation comprises: about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof; dodecyl maltoside; sodium chloride; optionally sodium metabisulfite; optionally ethylenediaminetetraacetic acid (EDTA) or disodium salt thereof; optionally benzalkonium chloride; hydrochloric acid, sodium hydroxide, or both, in amounts to adjust the pH to a final pH between about 3.0 and about 5.5; and water; in a volume between about 100 μL and about 140 μL.

In some embodiments, each intranasally delivered dose of the pharmaceutical formulation comprises: about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof; about 2.0 mg/mL to about 3.0 mg/mL of dodecyl maltoside; about 5 to about 10 mg/mL of sodium chloride as an excipient; hydrochloric acid, sodium hydroxide, or both, in amounts to adjust the pH to a final pH between about 3.0 and about 5.5; optionally about 0.1 mg/mL to about 4.0 mg/mL of ethylenediaminetetraacetic acid (EDTA) or disodium salt thereof; optionally about 0.1 mg/mL to about 0.8 mg/mL of benzalkonium chloride as an excipient; optionally about 0.01 mg/mL to about 0.1 mg/mL of sodium metabisulfite as an excipient; and water; in a volume between about 100 μL and about 140 μL.

In some embodiments, each intranasally delivered dose of the pharmaceutical formulation comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; dodecyl maltoside; sodium chloride; optionally sodium metabisulfite; optionally ethylenediaminetetraacetic acid (EDTA) or disodium salt thereof; optionally benzalkonium chloride; hydrochloric acid, sodium hydroxide, or both, in amounts to adjust the pH to a final pH between about 3.0 and about 5.5; and water; in a volume between about 100 μL and about 140 μL.

In some embodiments, each intranasally delivered dose of the pharmaceutical formulation comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; about 2.0 mg/mL to about 3.0 mg/mL of dodecyl maltoside; about 5 to about 10 mg/mL of sodium chloride as an excipient; hydrochloric acid, sodium hydroxide, or both, in amounts to adjust the pH to a final pH between about 3.0 and about 5.5; optionally about 0.1 mg/mL to about 4.0 mg/mL of ethylenediaminetetraacetic acid (EDTA) or disodium salt thereof; optionally about 0.1 mg/mL to about 0.8 mg/mL of benzalkonium chloride as an excipient; optionally about 0.01 mg/mL to about 0.1 mg/mL of sodium metabisulfite as an excipient; and water; in a volume between about 100 μL and about 140 μL.

In some embodiments, each dose of the the nasal spray is intranasally administered with a nasal spray device; and one actuation of the nasal spray device delivers about 100 μL of the pharmaceutical formulation.

In some embodiments, each intranasally administered dose comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; dodecyl maltoside; sodium chloride; optionally sodium metabisulfite; optionally ethylenediaminetetraacetic acid (EDTA) or disodium salt thereof; optionally benzalkonium chloride; hydrochloric acid, sodium hydroxide, or both, in amounts to adjust the pH to a final pH between about 3.0 and about 5.5; and water; in a volume between about 100 μL.

In some embodiments, each intranasally administered dose comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; about 2.0 mg/mL to about 3.0 mg/mL of dodecyl maltoside; about 5 to about 10 mg/mL of sodium chloride as an excipient; hydrochloric acid, sodium hydroxide, or both, in amounts to adjust the pH to a final pH between about 3.0 and about 5.5; optionally about 0.1 mg/mL to about 4.0 mg/mL of ethylenediaminetetraacetic acid (EDTA) or disodium salt thereof; optionally about 0.1 mg/mL to about 0.8 mg/mL of benzalkonium chloride as an excipient; optionally about 0.01 mg/mL to about 0.1 mg/mL of sodium metabisulfite as an excipient; and water; in a volume between about 100 μL.

In another aspect, described herein is a nasal spray pharmaceutical formulation comprising between about 0.10 mg and about 5.0 mg of epinephrine, or a salt thereof. In another aspect, described herein is a nasal spray pharmaceutical formulation comprising between about 0.40 mg and about 2.4 mg of epinephrine, or a salt thereof, in a single dose of the nasal spray pharmaceutical formulation. In another aspect, described herein is a nasal spray pharmaceutical formulation comprising between about 0.10 mg and about 5.0 mg of epinephrine, or a salt thereof, in a single dose nasal spray pharmaceutical formulation. In some embodiments, the nasal spray pharmaceutical formulation comprises between about 0.40 mg and about 2.0 mg of epinephrine, or a salt thereof. In some embodiments, the nasal spray pharmaceutical formulation comprises between about 0.40 mg and about 1.8 mg of epinephrine, or a salt thereof. In some embodiments, a single dose of the nasal spray pharmaceutical formulation comprises between about 0.5 mg and about 2.0 mg of epinephrine, or a salt thereof. In some embodiments, a single dose of the nasal spray pharmaceutical formulation comprises between about 0.5 mg and about 1.5 mg of epinephrine, or a salt thereof. In some embodiments, a single dose of the nasal spray pharmaceutical formulation comprises between about 0.5 mg and about 0.7 mg of epinephrine, or a salt thereof. In some embodiments, a single dose of the nasal spray pharmaceutical formulation comprises about 1.0 mg of epinephrine, or a salt thereof. In some embodiments, a single dose of the nasal spray pharmaceutical formulation comprises between about 1.3 mg and about 1.5 mg of epinephrine, or a salt thereof. In some embodiments, intranasal administration of a single dose of the nasal spray pharmaceutical formulation to a subject provides a plasma epinephrine concentration that are efficacious for the treatment of an acute hypersensitivity reaction. In some embodiments, the nasal spray pharmaceutical formulation is an aqueous solution, aqueous suspension, aqueous emulsion, non-aqueous solution, non-aqueous suspensions, non-aqueous emulsion, or dry powder.

In one aspect, described herein is a nasal spray formulation comprising between about 0.40 mg and about 2.4 mg per dose of epinephrine, or a salt thereof, dispensed from the device. In some embodiments, described herein is a nasal spray formulation comprising between about 0.5 mg and about 2.0 mg of epinephrine, or a salt thereof, per dose dispensed from the device; between about 0.5 mg and about 1.5 mg of epinephrine, or a salt thereof, per dose dispensed from the device; between about 0.5 mg and about 0.7 mg of epinephrine, or a salt thereof, per dose dispensed from the device; about 1.0 mg of epinephrine, or a salt thereof, per dose dispensed from the device; or between about 1.3 mg and about 1.5 mg of epinephrine, or a salt thereof, per dose dispensed from the device. In some embodiments, a single dose of the nasal spray formulation when administered intranasally provides plasma epinephrine concentrations that are efficacious for the treatment of an acute hypersensitivity reaction. In some embodiments, the epinephrine or salt thereof is present in the pharmaceutical formulation in an amount efficacious for the treatment of an acute hypersensitivity reaction. In some embodiments, the nasal spray formulation is an aqueous solution, aqueous suspension, aqueous emulsion, non-aqueous solution, non-aqueous suspension or non-aqueous emulsion.

In some embodiments, the nasal spray formulation comprises between about 1 mg/mL and about 40 mg/mL of epinephrine, or a salt thereof, per dose. In some embodiments, the nasal spray formulation comprises between about 5 mg/mL and about 40 mg/mL of epinephrine, or a salt thereof, per dose. In some embodiments, the nasal spray formulation comprises between about 1 mg/mL and about 20 mg/mL of epinephrine, or a salt thereof, per dose. In some embodiments, the nasal spray formulation comprises between about 3 mg/mL and about 20 mg/mL of epinephrine, or a salt thereof, per dose. In some embodiments, the nasal spray formulation comprises between about 3 mg/mL and about 15 mg/mL of epinephrine, or a salt thereof, per dose. In some embodiments, the nasal spray formulation comprises about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof, per dose. In some embodiments, a dose of the nasal spray formulation comprises about 100 μL of the nasal spray epinephrine formulation described herein.

In some embodiments, a nasal spray formulation described herein comprises about 1 mg/mL to about 40 mg/mL of epinephrine, or a salt thereof. In some embodiments, a nasal spray formulation described herein comprises about 1 mg/mL to about 20 mg/mL of epinephrine, or a salt thereof. In some embodiments, a nasal spray formulation described herein comprises about 1 mg/mL to about 18 mg/mL of epinephrine, or a salt thereof. In some embodiments, a nasal spray formulation described herein comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof. In some embodiments, a nasal spray formulation described herein comprises about 3 mg/mL, about 5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, about 15 mg/mL, about 15.5 mg/mL, about 16 mg/mL, about 16.5 mg/mL, about 17 mg/mL, about 17.5 mg/mL, about 18 mg/mL, about 18.5 mg/mL, about 19 mg/mL, about 19.5 mg/mL, about 20 mg/mL, about 20.5 mg/mL, about 21 mg/mL, about 21.5 mg/mL, about 22 mg/mL, about 22.5 mg/mL, about 23 mg/mL, about 23.5 mg/mL, about 24 mg/mL, about 24.5 mg/mL, about 25 mg/mL, about 25.5 mg/mL, about 26 mg/mL, about 26.5 mg/mL, about 27 mg/mL, about 27.5 mg/mL, about 28 mg/mL, about 28.5 mg/mL, about 29 mg/mL, about 29.5 mg/mL, about 30 mg/mL, about 30.5 mg/mL, about 31 mg/mL, about 31.5 mg/mL, about 32 mg/mL, about 32.5 mg/mL, about 33 mg/mL, about 33.5 mg/mL, about 34 mg/mL, about 34.5 mg/mL, about 35 mg/mL, about 35.5 mg/mL, about 36 mg/mL, about 36.5 mg/mL, about 37 mg/mL, about 37.5 mg/mL, about 38 mg/mL, about 38.5 mg/mL, about 39 mg/mL, about 39.5 mg/mL, about 40 mg/mL, about 40.5 mg/mL, about 41 mg/mL, about 41.5 mg/mL, about 42 mg/mL, about 42.5 mg/mL, about 43 mg/mL, about 43.5 mg/mL, about 44 mg/mL, about 44.5 mg/mL, about 45 mg/mL, about 45.5 mg/mL, about 46 mg/mL, about 46.5 mg/mL, about 47 mg/mL, about 47.5 mg/mL, about 48 mg/mL, about 48.5 mg/mL, about 49 mg/mL, about 49.5 mg/mL, or about 50 mg/mL, of epinephrine, or a salt thereof. In some embodiments, a nasal spray formulation described herein comprises about 10 mg/mL of epinephrine, or a salt thereof. In some embodiments, a nasal spray formulation described herein comprises about 20 mg/mL of epinephrine, or a salt thereof. In some embodiments, a nasal spray formulation described herein comprises about 15 mg/mL, about 15.5 mg/mL, about 16 mg/mL, about 16.5 mg/mL, about 17 mg/mL, about 17.5 mg/mL, about 18 mg/mL, about 18.5 mg/mL, about 19 mg/mL, about 19.5 mg/mL, about 20 mg/mL, about 20.5 mg/mL, about 21 mg/mL, about 21.5 mg/mL, about 22 mg/mL, about 22.5 mg/mL, about 23 mg/mL, about 23.5 mg/mL, about 24 mg/mL, about 24.5 mg/mL, or about 25 mg/mL of epinephrine, or a salt thereof. In some embodiments, a nasal spray formulation described herein comprises about 6 mg/mL to about 8 mg/mL of epinephrine, or a salt thereof. In some embodiments, a nasal spray formulation described herein comprises about 13 mg/mL to about 15 mg/mL of epinephrine, or a salt thereof. In some embodiments, a dose of the nasal spray formulation comprises about 100 μL of the nasal spray epinephrine formulation described herein. In some embodiments, about 100 μL of the nasal spray epinephrine formulation described herein with one actuation of a nasal spray device.

In some embodiments, a dose of about 100 μL of the nasal spray formulation described herein comprises 1 mg/mL to about 40 mg/mL of epinephrine, or a salt thereof. In some embodiments, a dose of about 100 μL of the nasal spray formulation described herein comprises about 1 mg/mL to about 20 mg/mL of epinephrine, or a salt thereof.

In some embodiments, the nasal spray formulation comprises one or more absorption enhancers.

In some embodiments, the nasal spray formulation provides intramuscular (IM)-injection-like pharmacokinetics when IM-injection is dosed in the lateral thigh, or subcutaneous (SC)-like absorption or in between.

In some embodiments, the nasal spray formulation provides intramuscular (IM)-injection-like absorption.

In some embodiments, the nasal spray formulation provides subcutaneous (SC)-like absorption and the SC pharmacokinetic profile has a Cₘₐₓ of at least 100 μg/mL and AUC₀₋₂₄₀ₘᵢₙ of 150 h*pg/mL.

In some embodiments, intranasal administration of a single dose of the nasal spray pharmaceutical formulation to a subject provides intramuscular (IM)-injection-like absorption.

In some embodiments, the nasal spray formulation comprises between about 0.5 and about 1.1 molar equivalents of acid to each mole of epinephrine. In some embodiments, the acid is adipic acid, ammonium chloride, citric acid, acetic acid, hydrochloric acid, lactic acid, phosphoric acid, propionic acid, sulfuric acid or tartaric acid. In some embodiments, the acid is hydrochloric acid.

In some embodiments, no base is added to the nasal spray formulation during its preparation. In some embodiments, the nasal spray formulation has a pH between about 2.0 and about 6.0. In some embodiments, the nasal spray formulation has a pH of about 4.0.

In some embodiments, the nasal spray formulation comprises between about 5 mg/mL and about 40 mg/mL per dose epinephrine, or a salt thereof. In some embodiments, the nasal spray formulation comprises between about 0.9 mg and about 2.40 mg per dose dispensed from the device of epinephrine, or a salt thereof. In some embodiments, the nasal spray formulation comprises between about 0.5 mg and about 2.0 mg per dose dispensed from the device of epinephrine, or a salt thereof. In some embodiments, the nasal spray formulation comprises between about 0.9 mg and about 1.5 mg per dose dispensed from the device of epinephrine, or a salt thereof. In some embodiments, the nasal spray formulation comprises between about 0.75 mg and about 1.5 mg per dose dispensed from the device of epinephrine, or a salt thereof. In some embodiments, the nasal spray formulation comprises between about 0.45 mg and about 1.15 mg per dose dispensed from the device of epinephrine, or a salt thereof. In some embodiments, the nasal spray formulation comprises between about 1.0 mg and about 2.0 mg per dose dispensed from the device of epinephrine, or a salt thereof. In some embodiments, the nasal spray formulation comprises between about 0.5 mg and about 2.0 mg of epinephrine, or a salt thereof, per dose dispensed from the device. In some embodiments, the nasal spray formulation comprises between about 0.5 mg and about 1.5 mg of epinephrine, or a salt thereof, per dose dispensed from the device. In some embodiments, the nasal spray formulation comprises between about 0.5 mg and about 0.7 mg of epinephrine, or a salt thereof, per dose dispensed from the device. In some embodiments, the nasal spray formulation comprises about 1.0 mg of epinephrine, or a salt thereof, per dose dispensed from the device. In some embodiments, the nasal spray formulation comprises between about 1.3 mg and about 1.5 mg of epinephrine, or a salt thereof, per dose dispensed from the device.

In some embodiments, the nasal spray pharmaceutical formulation comprises one or more absorption enhancement agents; and optionally one or more agents selected from isotonicity agents; stabilizing agents; preservatives; taste-masking agents; viscosity modifiers; antioxidants; buffers and pH adjustment agents; wherein the pH of the nasal spray pharmaceutical formulation is between about 2.0 and about 6.0.

In some embodiments, the nasal spray pharmaceutical formulation has a pH between about 3.0 and about 5.0. In some embodiments, the nasal spray pharmaceutical formulation has a pH of about 4.0. In some embodiments, the nasal spray pharmaceutical formulation comprises pH adjustment agents. In some embodiments, the pH adjustment agent is an acid, a base, a buffer, or a combination thereof. In some embodiments, the acid is adipic acid, ammonium chloride, citric acid, acetic acid, hydrochloric acid, lactic acid, phosphoric acid, propionic acid, sulfuric acid or tartaric acid; the base is sodium hydroxide, sodium citrate, sodium bicarbonate, sodium carbonate; and the buffer is a phosphate buffer, acetate buffer, or citrate buffer. In some embodiments, the nasal spray pharmaceutical formulation comprises between about 0.5 and about 1.1 molar equivalents of acid to each mole of epinephrine. In some embodiments, the acid is hydrochloric acid.

In some embodiments, the nasal spray formulation comprises one or more absorption enhancers selected from dodecyl maltoside, benzalkonium chloride, oleic acid, or salt thereof, polysorbate 20, polysorbate 80, and sodium lauryl sulfate.

In some embodiments, the formulation comprises one or more absorption enhancers selected from alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl camitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, or salt thereof, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol β-D-glucoside, sodium lauryl sulfate, sucrose cocoate, taurocholic acid, taurodeoxycholic acid, taurodihydrofusidic acid, thymol, tricaprylin, triolein, and alkylsaccharides.

In some embodiments, the formulation comprises one or more absorption enhancers selected from dodecyl maltoside, benzalkonium chloride, oleic acid, or salt thereof, polysorbate 20, polysorbate 80, and sodium lauryl sulfate.

In some embodiments, the formulation comprises a preservative. In some embodiments, the preservative is benzalkonium chloride.

In some embodiments, the nasal spray pharmaceutical formulation comprises an isotonicity agent. In some embodiments, the isotonicity agent is dextrose, glycerin, mannitol, potassium chloride, or sodium chloride. In some embodiments, the isotonicity agent is sodium chloride.

In some embodiments, the nasal spray formulation additionally comprises a stabilizing agent. In some embodiments, the stabilizing agent is ethylenediaminetetraacetic acid (EDTA) or a salt thereof. In some embodiments, the EDTA is disodium EDTA. In some embodiments, the nasal spray formulation comprises from about 0.001% (w/v) to about 1% (w/v) of disodium EDTA.

In some embodiments, the nasal spray formulation additionally comprises a preservative. In some embodiments, the preservative is benzalkonium chloride.

In some embodiments, the nasal spray formulation comprises one or more absorption enhancers selected from alkylglycosides, benzalkonium chloride, oleic acid, or salt thereof, polysorbate 20, polysorbate 80, sodium lauryl sulfate, cyclodextrins, medium and long chain fatty acids, or salts thereof, saturated and unsaturated fatty acids, or salts thereof, alcohol, glycerin, propylene glycol, PEG 300/400, and benzyl alcohol.

In some embodiments, the nasal spray formulation further comprises an antioxidant. In some embodiments, the nasal spray formulation further comprises an antioxidant selected from alpha tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof.

In some embodiments, the nasal spray formulation further comprises synergists with the antioxidants selected from citric acid monohydrate, tartaric acid, thymol, tocopherol (alpha tocopherol), tocopherasol, vitamin E and vitamin E polyethylene glycol succinate and a combination thereof.

In some embodiments, the nasal spray formulation further comprises permeation enhancers selected from alcohol, arachidonic acid, benzethonium chloride, benzethonium bromide, benzalkonium chloride, capric acid, caproic acid, carvone, cetylpyridium chloride, chitosans, citric acid, 6-cyclohexyl-1-hexyl-β-D-maltopyranoside, n-decyl-β-D-maltopyranoside, dimethyl sulfoxide, dodecyl dimethyl aminopropionate, 1-O-n-Dodecyl-β-D-maltopyranoside, dodecylpolyethyleneglycolether, edetate disodium dihydrate, enanthic acid, glyceryl monooleate, glyceryl monostearate, glycofurol, isopropyl myristate, isopropyl palmitate, pelargonic acid, lanolin, lauric acid, light mineral oil, limonene, linoleic acid, lysine, menthol, myristic acid, myristyl alcohol, oleic acid, oleyl alcohol, palmitic acid, peppermint oil, polyoxyethylene alkyl ethers, polyoxylglycerides, polysorbates, pyrrolidone, sodium caprate, sodium desoxycholate, sodium deoxyglycolate, sodium glycocholate, sodium hydroxybenzoyal amino caprylate, sodium lauryl sulfate, sodium taurocholate, stearic acid, thymol, tricaprylin, triolein, undecylenic acid, and a combination thereof.

In some embodiments, the nasal spray formulation comprises: about 0.0010% to 1% of any one of the antioxidants described herein, or a combination of any one of the antioxidants described herein.

In some embodiments, the nasal spray formulation comprises a buffering agent. Buffering agents include, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodiumhydroxide, sodium lactate, and triethanolamine.

In one aspect, provided herein is a method of treatment of a condition mediated by adrenergic receptors comprising the intranasal administration of any one of the formulations as described herein. In some embodiments, the condition is chosen from a type-1 hypersensitivity reaction (systemic allergic reaction), an acute asthmatic attack, cardiac arrest, and Stokes-Adams Syndrome. In some embodiments, the condition is a type-1 hypersensitivity reaction (systemic allergic reaction). In some embodiments, the type 1 hypersensitivity reaction is chosen from allergic asthma, allergic conjunctivitis, allergic rhinitis, anaphylaxis, angioedema, urticaria, eosinophilia, drug allergy and food allergy. In some embodiments, the drug allergy is an antibiotic allergy.

In some embodiments, the nasal spray formulation is a pharmaceutical formulation.

In some embodiments, the epinephrine or salt thereof is present in the nasal spray formulation in an amount efficacious for the treatment of an acute hypersensitivity reaction.

In some embodiments, intranasal administration of a single dose of the nasal spray pharmaceutical formulation to a subject provides a plasma epinephrine concentration that is efficacious for the treatment of an acute hypersensitivity reaction. In some embodiments, a single dose of the nasal spray pharmaceutical formulation comprises between about 0.5 mg and about 2.0 mg of epinephrine, or a salt thereof. In some embodiments, a single dose of the nasal spray pharmaceutical formulation comprises between about 0.5 mg and about 1.5 mg of epinephrine, or a salt thereof. In some embodiments, a single dose of the nasal spray pharmaceutical formulation comprises between about 0.5 mg and about 0.7 mg of epinephrine, or a salt thereof. In some embodiments, a single dose of the nasal spray pharmaceutical formulation comprises about 1.0 mg of epinephrine, or a salt thereof. In some embodiments, a single dose of the nasal spray pharmaceutical formulation comprises between about 1.3 mg and about 1.5 mg of epinephrine, or a salt thereof. In some embodiments, the formulation is an aqueous solution, aqueous suspension, aqueous emulsion, non-aqueous solution, non-aqueous suspension, non-aqueous emulsion, pressurized metered-dose inhalers or dry powder.

In some embodiments, the nasal spray formulation is an aqueous solution, aqueous suspensions, aqueous emulsion, non-aqueous solution, non-aqueous suspension or non-aqueous emulsion.

In some embodiments, the nasal spray formulation has intramuscular (IM)-injection-like pharmacokinetics when IM-injection is dosed in the lateral thigh, or subcutaneous (SC)-like absorption or in between.

In some embodiments, the nasal spray formulation has subcutaneous (SC)-like absorption and the SC pharmacokinetic profile has a Cmax of at least 100 μg/mL and AUC0-240 min of 150 h*pg/mL.

In some embodiments, the nasal spray formulation has intramuscular (IM)-injection-like absorption.

In some embodiments, the nasal spray formulation comprises an absorption enhancer.

In some embodiments, the pH of the nasal spray pharmaceutical formulation is between about 2.0 and about 6.0. In some embodiments, the nasal spray pharmaceutical formulation has a pH between about 3.0 and about 5.0. In some embodiments, the nasal spray pharmaceutical formulation has a pH of about 4.0.

In some embodiments, the nasal spray pharmaceutical formulation comprises pH adjustment agents. In some embodiments, the pH adjustment agent is an acid, a base, a buffer, or a combination thereof. In some embodiments, the acid is adipic acid, ammonium chloride, citric acid, acetic acid, hydrochloric acid, lactic acid, phosphoric acid, propionic acid, sulfuric acid or tartaric acid; the base is sodium hydroxide, sodium citrate, sodium bicarbonate, sodium carbonate; and the buffer is a phosphate buffer, acetate buffer, or citrate buffer.

In some embodiments, the nasal spray formulation comprises between about 0.5 and about 1.1 molar equivalents of acid to each mole of epinephrine. In some embodiments, the acid is adipic acid, ammonium chloride, citric acid, acetic acid, hydrochloric acid, lactic acid, phosphoric acid, propionic acid, sulfuric acid or tartaric acid. In some embodiments, the acid is hydrochloric acid. In some embodiments, no base is added to the formulation during its preparation. In some embodiments, the nasal spray formulation has a pH between about 2.0 and about 6.0. In some embodiments, the nasal spray formulation has a pH of about 4.0.

In some embodiments, the nasal spray formulation comprises between about 5 mg/mL and about 40 mg/mL per dose epinephrine, or a salt thereof. In some embodiments, the nasal spray formulation comprises between about 0.40 mg and about 2.40 mg per dose dispensed from the device of epinephrine, or a salt thereof. In some embodiments, the nasal spray formulation comprises between about 0.9 mg and about 2.40 mg per dose dispensed from the device of epinephrine, or a salt thereof. In some embodiments, the nasal spray formulation comprises between about 0.5 mg and about 2.0 mg per dose dispensed from the device of epinephrine, or a salt thereof. In some embodiments, the nasal spray formulation comprises between about 0.9 mg and about 1.5 mg per dose dispensed from the device of epinephrine, or a salt thereof. In some embodiments, the nasal spray formulation comprises between about 0.75 mg and about 1.5 mg per dose dispensed from the device of epinephrine, or a salt thereof. In some embodiments, the nasal spray formulation comprises between about 0.45 mg and about 1.15 mg per dose dispensed from the device of epinephrine, or a salt thereof. In some embodiments, the nasal spray formulation comprises between about 1.0 mg and about 2.0 mg per dose dispensed from the device of epinephrine, or a salt thereof.

In some embodiments, the nasal spray formulation comprises one or more absorption enhancers selected from alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl camitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol β-D-glucoside, sodium lauryl sulfate, sucrose cocoate, taurocholic acid, taurodeoxycholic acid, taurodihydrofusidic acid, thymol, tricaprylin, triolein, and alkylsaccharides.

In some embodiments, the nasal spray formulation comprises one or more absorption enhancers selected from dodecyl maltoside, benzalkonium chloride, oleic acid, or salt thereof, polysorbate 20, polysorbate 80, and sodium lauryl sulfate.

In some embodiments, the nasal spray formulation additionally comprises a stabilizing agent. In some embodiments, the stabilizing agent is ethylenediaminetetraacetic acid (EDTA) or a salt thereof. In some embodiments, the EDTA is disodium EDTA. In some embodiments, the EDTA is present in an amount that is from about 0.0010% to about 10%.

In some embodiments, the nasal spray formulation additionally comprises a preservative. In some embodiments, the preservative is benzalkonium chloride.

In one aspect, described herein is a method of treatment of a condition mediated by adrenergic receptors comprising the intranasal administration of any one of the formulation described herein. In some embodiments, the condition is chosen from a type-1 hypersensitivity reaction (systemic allergic reaction), an acute asthmatic attack, cardiac arrest, and Stokes-Adams Syndrome. In some embodiments, the condition is a type-1 hypersensitivity reaction (systemic allergic reaction). In some embodiments, the type 1 hypersensitivity reaction is chosen from allergic asthma, allergic conjunctivitis, allergic rhinitis, anaphylaxis, angioedema, urticaria, eosinophilia, drug allergy and food allergy. In some embodiments, the drug allergy is an antibiotic allergy.

In another aspect, described herein is a method of treatment of anaphylaxis comprising the intranasal administration of an intranasal formulation of epinephrine in an amount of about 2.0 mg. In some embodiments, the nasal pharmaceutical formulation comprises between about 0.5 mg and about 1.5 mg of epinephrine, or a salt thereof. In some embodiments, the nasal pharmaceutical formulation comprises between about 0.5 mg and about 0.7 mg of epinephrine, or a salt thereof. In some embodiments, the nasal pharmaceutical formulation comprises about 1.0 mg of epinephrine, or a salt thereof. In some embodiments, the nasal pharmaceutical formulation comprises about 2.0 mg of epinephrine, or a salt thereof. In some embodiments, the nasal pharmaceutical formulation comprises between about 1.3 mg and about 1.5 mg of epinephrine, or a salt thereof.

Articles of manufacture, which include packaging material, a nasal spray formulation described herein within the packaging material, and a label that indicates that the nasal spray formulation is used for the treatment of any of the conditions described herein (e.g. anaphylaxis) are provided.

Other objects, features and advantages of the compositions and methods described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF FIGURES

FIG. 1: Mean Change from Baseline in Systolic Blood Pressure (mm Hg)

FIG. 2: Mean Change from Baseline in Heart Rate (bpm)

FIG. 3: Mean Change from Baseline in Diastolic Blood Pressure (mmHg)

FIG. 4: Mean epinephrine plasma concentrations of repeat doses of 2 mg neffy compared to repeat doses 0.3 mg IM injection with and without nasal allergen challenge (NAC).

### DETAILED DESCRIPTION

Disclosed herein are methods and formulations useful for the treatment of anaphylaxis and other conditions, comprising administering an intranasal formulation of epinephrine. Also provided are devices adapted for nasal delivery of a pharmaceutical formulation to a patient, including single, bi- and multidose delivery comprising a therapeutically effective amount of epinephrine and pharmaceutically acceptable salts thereof.

### Epinephrine and Type-I Hypersensitivity Reactions, Including Anaphylaxis

Anaphylaxis is a systemic and life-threatening allergic reaction characterized by anaphylactic shock associated with a critical decrease in blood pressure and deterioration in consciousness. It is the most severe form of allergic reaction and is almost always unexpected. Epinephrine is the medication of first choice for the treatment of anaphylaxis because it is the only medication that reduces hospitalization and death. Death can occur rapidly and unpredictably in anaphylaxis, and therefore epinephrine should be administered promptly to achieve peak concentrations rapidly in plasma and tissues. Delay in treatment may result in death by airway obstruction or vascular collapse.

Epinephrine has long been used in the treatment of type I hypersensitivity reactions, including anaphylaxis. Epinephrine auto injectors have been approved by the U.S. Food and Drug Administration (FDA) for the treatment of Type 1 hypersensitivity reactions, including anaphylaxis, as early as 1987 and include EpiPen® (IM & SC routes; 0.15 & 0.3 mg), Twinject® (IM & SC routes; 0.15 & 0.3 mg), Adrenaclick® (IM & SC routes; 0.15 & 0.3 mg), Auvi-Q® (IM & SC routes; 0.1, 0.15 & 0.3 mg), Symjepi® (IM & SC routes; 0.15 & 0.3 mg), Teva EpiPen® (IM & SC routes; 0.15 & 0.3 mg). All injection products demonstrate efficacy despite different PK.

TABLE A
Epinephrine Injection Products

| | | Mean Study | | |
|---|---|---|---|---|
| | | Cmax | Median or Mean | Study Tmax |
| Treatment | N | (pg/mL) | Study Tmax(min) | Range (min) |
| EpiPen 0.3 mg | 507 | 288-869 | 5-40 | 1-240 |
| IM 0.3 mg | 381 | 209-489 | 30 to 60 | 3-360 |
| Auvi-Q 0.3 mg | 67 | 486 | 20 | 5-60 |
| Symjepi 0.3 mg | 88 | 337-438 | 30 | 4-240 |
| SC 0.3 mg | 36 | 246 | 45 | 4-180 |
| Total Range | | 209 to 869 | 5 to 60 | 1 to 360 |

Despite the long-standing use of epinephrine auto injectors in the management of anaphylaxis, epinephrine is known to have a narrow therapeutic window (see Simons, et al., J. Aller. & Clin. Immunol., 2006, Vol. 117, No. 2, pp 367-377). Accordingly, the life-saving pharmacologic effects of epinephrine, including vasoconstriction, decreased mucosal edema, bronchodilation, and decreased release of histamine, tryptase, and other mediators of inflammation, cannot be divorced from pharmacologic effects such as pallor, anxiety, tremor, and palpitations, which are perceived as adverse effects.

On the other hand, serious adverse effects are usually liked to epinephrine overdose, e.g., through accidental intravenous injection.

Furthermore, epinephrine auto injectors are considered inconvenient and cumbersome, particularly for paediatric patients. The patient themselves, or a caregiver, must use the product to intramuscularly or subcutaneously inject epinephrine in an emergency setting. Due to this route of administration, the act of injection is one of the common reasons cited for the lack of use of epinephrine auto injectors.

Intranasal epinephrine has a long history of use in low doses as a decongestant and as a vasoconstrictor, often formulated combination with anaesthetic, in sinus and nasal surgery.

Epinephrine's powerful vasoconstrictor effects are important in its ability to relieve hypotensive shock which is a significant symptom of anaphylaxis. These vasoconstriction properties were also reported to be a key factor in the poor absorption of intranasally delivered epinephrine.

Historically, epinephrine has been difficult to formulate as an intranasal solution for systemic delivery. See, e.g., Srisawat C et al., “A preliminary study of intranasal epinephrine administration as a potential route for anaphylaxis treatment,” Asian Pac J Allergy Immunol, 2016 Mar; 34(1):38-43. Srisawat showed that significant systemic absorption of epinephrine via the IN route was observed only at 5 mg and the pharmacokinetic parameters of IN epinephrine even at 5 mg were also not significantly different from those of the IM epinephrine group (see Table B, below).

TABLE B
Intramuscular and Intranasal Administraton
of Epinephrine (from Srisawat (2016).

| | Intramuscular (IM) | Intranasal (IN) |
|---|---|---|
| Mean ± SD | Epinephrine 0.3 mg | Epinephrine 5 mg |
| C_{baseline} (pg/mL) | 35 ± 23 | 8 ± 6 |
| Cₘₐₓ (pg/mL) | 309 ± 88 | 386 ± 152 |
| Tₘₐₓ (min) | 67 ± 43 | 70 ± 17 |
| AUC_{0-120 min} (ng*min/mL) | 18.3 ± 9.3 | 19.4 ± 12.1 |

Srisawat C et al. demonstrated that no blood level of epinephrine at the intranasal dose level of 2.5 mg and below (i.e., 0.3 mg, 0.6 mg, 1.25 mg, or 2.5 mg).

Furthermore, even at a dose of 5 mg, Srisawat was not able to make an intranasal formulation that could achieve a higher plasma concentration than intramuscular epinephrine delivered by auto injector at any time point before about 60 minutes, thus absorption during the critical early time points was delayed when rapid absorption is needed to stop the systemic allergic reaction (anaphylaxis). This is potentially detrimental in serious conditions such as anaphylaxis where immediate treatment, and thus injection-like pharmacokinetics, are desirable. The PK profile of IM injection into the thigh is considered the optimal dosing method by literature given that the higher vascularity of the leg muscle allows for more rapid absorption and distribution of the epinephrine providing a rapid increase in plasma levels to stop the anaphylaxis reaction much sooner than other routes of administration. Srisawat's 5 mg formulation, in contrast to intramuscular epinephrine delivered by auto injector, cleared from the plasma almost entirely in about two hours. Finally, epinephrine is known to be associated with dose-related cardiac side effects including myocardial infarction, at doses as low as 0.3 to 0.5 mg intramuscularly; accordingly, doses as high as 5 mg would likely be risky in the general population if nasal conditions existed that may allow excessive absorption. Thus, lower dose preparations that would avoid such risks are preferred as a safer nasal preparation.

Srisawat notes that epinephrine's vasoconstriction effect prevents its own systemic absorption when administered intranasally, and suggests overcoming this effect by coformualting with drugs possessing vasodilatation effect, e.g. alpha-adrenergic receptor antagonists such as phentolamine, to counter the vasoconstriction effect of epinephrine. Coadminitering or coformulating intranasal epinephrine formulations with a vasodilator (phentolamine, to stop inhibition of mucosal transport by gross vasodilation) and/or a reversible topically-acting COMT inhibitor (to prevent epinephrine enzymatic degradation at the nasal mucosa), have been attempted with little success.

The IN epinephrine formulations described herein achieve systemic absorption via the intranasal route, thereby overcoming the major technical challenge that was required to overcome in order to achieve systemic absorption via the intranasal route.

The IN epinephrine formulations described herein represent a safe, effective, and more convenient alternative to the current standard of care for anaphylaxis, i.e., epinephrine injection.

More specifically, the IN epinephrine formulations described herein avoid both the substantial risk of intravenous (IV) bolus injection associated with epinephrine injection products and substantial risk of accidental epinephrine overdose associated with higher dosage IN formulations.

The advantage of the IN epinephrine formulations described herein is that the intranasal absorption of epinephrine is improved, such that a suitable pharmacokinetic (PK) profile for treatment of anaphylaxis is obtained (i.e., providing a rapid Tₘₐₓ while maintaining Cₘₐₓ and AUC (exposure) levels that are tolerable and safe) with a safer low dose of epinephrine (to reduce the risk of accidental epinephrine overdose, e.g., on repeat administration).

### More Severe Cases

If the response to the first epinephrine injection is inadequate, it can be repeated once or twice at 5- to 15-minute intervals. A third dose is needed infrequently. Subsequent epinephrine doses are needed for severe or rapidly progressive anaphylaxis and for failure to respond to the initial injection because of delayed injection of the initial dose, inadequate initial dose, or administration through a suboptimal route (Simons K J, Simons F E R. Epinephrine and its use in anaphylaxis: current issues. Curr Opin Allergy Clin Immunol. 2010; 10(4):354-361). Subsequent doses also might be needed in biphasic anaphylaxis, defined as recurrence of symptoms hours after resolution of initial symptoms despite no further exposure to the trigger, which is reported in up to 110% of pediatric patients. Food-induced anaphylaxis is associated with biphasic anaphylaxis less often than is venom- or drug-induced anaphylaxis (Mehr S, Liew W K, Tey D, Tang M L. Clinical predictors for biphasic reactions in children presenting with anaphylaxis. Clin Exp Allergy. 2009; 39(9):1390-1396; ee S, Bellolio M F, Hess E P, Erwin P, Murad M H, Campbell R L. Time of onset and predictors of biphasic anaphylactic reactions: a systematic review and meta-analysis. J Allergy Clin Immunol Pract. 2015; 3(3):408-16.e1, e2)

Hypotension is a greater concern in severe cases or when treatment is delayed and disease progression occurs (Blazowski L, et al., A severity grading system of food-induced acute allergic reactions to avoid the delay of epinephrine administration. Ann Allergy Asthma Immunol. 2021 Oct; 127(4):462-470.e2; Ko B S, et al., Should adrenaline be used in patients with hemodynamically stable anaphylaxis?Incident case control study nested within a retrospective cohort study. Sci Rep. 2016 Feb. 3; 6:20168).

### Nasal Congestion

Although IN administration of epinephrine offers several advantages over IM injection, alterations in the nasal environment may influence drug absorption and delivery to target areas. Nasal congestion has been reported during anaphylactic episodes, and has the potential to interfere with IN epinephrine in the treatment of anaphylaxis (Bayat R, et al., Allergy Asthma C/in Immunol. 2014; 10:6; Soller L, et al, Allergy Asthma C/in Immunol. 2018; 14:64 20, 21). During an anaphylactic or allergic event, the release of inflammatory mediators, such as histamine, contribute to vasodilation (Naclerio R M, et al., Pathophysiology of nasal congestion. Int J Gen Med. 2010; 3:47-57). Increased vasodilation can result in edema and swelling of the nasal mucosa, impeding air flow and increasing nasal secretions. Alpha-adrenergic receptor agonists like epinephrine have known decongestant activity. During an allergic response, histamine release may counteract the vasoconstrictive effects of epinephrine. As a result, rhinitis symptoms may present additional barriers on the absorption of epinephrine from an intranasally administered IN formulation of epinephrine, wherein epinephrine is the only pharmaceutically active agent in the IN formulation. In some embodiments, some or all of an intranasally administered IN dose of epinephrine may be needed to counteract the local edema, congestion, rhinnohea, and/or epistaxis in the nasal cavity, thereby resulting in a fraction of the IN dose of epinephrine available to be systematically absorbed for treatment of the type 1 hypersensitivity reaction.

As shown herein, edema and congestion does not interfere with the absorption in IN epinephrine. Administration of a second IN dose in the same nostril provides a significantly enhanced epinephrine exposure over the first IN dose in patients with rhinitis symptoms. In some embodiments, the enhanced exposure of the second IN dose in the same nostril is due to reversal of the symptoms of rhinitis by the first dose.

Disclosed herein are intranasal formulations of epinephrine, and nasal spray devices comprising the formulations, that solve the problems of past attempts for intranasally administering epinephrine for the treatment of type 1 hypersensitivity reactions. Various aspects may contribute to the success of the formulations, devices, and methods of use disclosed herein.

For example, in certain embodiments, formulating epinephrine in an aqueous solution with the appropriate addition of molar equivalents of acid to each mole of said epinephrine helps to solubilize and stabilize the epinephrine. This allows the formulation to avoid the use of buffering agents commonly used in aqueous pharmaceutical compositions for injection, including phosphate, acetate, and citrate buffers, which are sometimes avoided in the nasal formulations disclosed herein. Other salts of epinephrine, such as epinephrine acetate, epinephrine hydrochloride, epinephrine tartrate, epinephrine bitartrate, epinephrine hydrogen tartrate and epinephrine borate can also be used to formulate aqueous solutions of epinephrine.

Certain embodiments of the formulations, devices, and methods of use disclosed herein offer advantages over epinephrine formulated in other ways. Epinephrine is considered a narrow therapeutic index drug. As a sympathomimetic catecholamine, epinephrine has a narrow therapeutic index and serious adverse reactions including cardiovascular and cerebrovascular reactions can be associated with its use. Nevertheless, the use epinephrine for this indication is lif e saving and the benefits of using it outweigh the potential safety risks. Intranasal delivery and formulation are suited for the safe, painless delivery of drugs such as epinephrine by consistent content uniformity, delivery amount and absorption, thereby minimizing serious adverse reactions including cardiovascular and cerebrovascular reactions that can be associated with its use via injection mechanisms. Shot weights have low variability and consistently deliver the labeled dose.

In one aspect, described herein is a pharmaceutical composition comprising: a) epinephrine; and b) an alkylglycoside; wherein the pharmaceutical composition is formulated for administration into the circulatory system of a subject via the intranasal, inhalation, or pulmonary, administration route. In some embodiments, described herein is a pharmaceutical composition comprising: a) epinephrine; and b) an alkylglycoside; wherein the pharmaceutical composition is a liquid formulated for intranasal delivery.

In some embodiments, the alkylglycoside has an alkyl chain including between 8 to 20 carbons. In some embodiments, the alkylglycoside is selected from the group consisting of undecyl maltoside, dodecyl maltoside, tridecyl maltoside, tetradecyl maltoside, sucrose mono-dodecanoate, sucrose mono-tridecanoate, and sucrose mono-tetradecanoate. In some embodiments, the alkylglycoside is dodecyl-beta-D-maltoside. In some embodiments, the alkylglycoside concentration is between about 0.001% and 10.0% (w/v). In some embodiments, the alkylglycoside concentration is between about 0.05% and 0.5% (w/v).

In some embodiments, the composition further comprises a membrane penetration-enhancing agent. In some embodiments, the membrane penetration-enhancing agent is a surfactant, a bile salt, a phospholipid, an alcohol, an enamine, a long-chain amphipathic molecule, a small hydrophobic molecule, sodium or a salicylic acid derivative, a glycerol ester of acetoacetic acid, a cyclodextrin, a medium-chain or long chain fatty acids, a chelating agent, an amino acid or salt thereof, an enzyme or combination thereof. In some embodiments, the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, hydroxyquinolone, sodiumhydroxide, and combinations thereof.

In some embodiments, the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, sodium hydroxide, and combinations thereof. In some embodiments, membrane penetration-enhancing agent is benzalkonium chloride, EDTA, or a combination thereof.

In some embodiments, the composition provides a Cmax for the epinephrine in a subject that is about 2 fold or greater as compared to administration without alkylglycoside.

In some embodiments, the composition provides a Tmax for the epinephrine in a subject that is about 2 fold or less as compared to administration without alkylglycoside.

In some embodiments, the composition provides a Tmax for the epinephrine of about 0.3 hours or less in a subject.

In some embodiments, the composition has a pH of about 2.0 to about 6.0. In some embodiments, the composition has a pH of about 2.0 to about 5.0. In some embodiments, the composition has a pH of about 3.0 to about 5.5. In some embodiments, the composition has a pH of about 2.0 to about 5.5. In some embodiments, the composition has a pH of about 2.5 to about 5.5.

In some embodiments, the composition has a pH of about 3.5 to about 5.5. In some embodiments, the composition has a pH of about 4.0 to about 5.5. In some embodiments, the composition has a pH of about 4.5 to about 5.5. In some embodiments, the composition has a pH of about 2.5 to about 5.0. In some embodiments, the composition has a pH of about 3.5 to about 5.0. In some embodiments, the composition has a pH of about 4.0 to about 5.0. In some embodiments, the composition has a pH of about 4.5 to about 5.0. In some embodiments, the composition has a pH of about 2.0 to about 5.5. In some embodiments, the composition has a pH of about 2.5 to about 4.5. In some embodiments, the composition has a pH of about 3.5 to about 4.5. In some embodiments, the composition has a pH of about 4.0 to about 4.5. In some embodiments, the composition has a pH of about 3.0 to about 4.5.

In another aspect, described herein is a method of increasing the bioavailability of epinephrine in a subject comprising administering to a subject a pharmaceutical composition comprising epinephrine and an alkylglycoside, thereby increasing the bioavailability of the epinephrine in the subject; wherein the pharmaceutical composition is formulated for administration into the circulatory system of a subject via the intranasal, inhalation, or pulmonary, administration route. In some embodiments, described herein is a method of increasing the bioavailability of epinephrine in a subject comprising administering to a subject a pharmaceutical composition comprising epinephrine and an alkylglycoside, thereby increasing the bioavailability of the epinephrine in the subject; wherein the pharmaceutical composition is a liquid formulated for intranasal delivery.

In some embodiments, increasing the bioavailability of epinephrine permits for lower dose amounts of epinephrine to be delivered intranasally and be efficacious for treating anaphylaxis. In some embodiments, exposure to larger doses of epinephrine can result in an epinephrine overdose.

There is increased interest and need in developing alternative non-invasive epinephrine dosage forms that provide epinephrine plasma concentrations equivalent to those obtained by epinephrine auto-injectors, available in a range of doses, have a long shelf-life, and be free from needle anxiety, the possibility of administration error, unintentional injection and injury. Epinephrine nasal dosage forms described herein offer the potential of being user-friendly, non-invasive alternatives for the first-aid emergency treatment of anaphylaxis in community settings.

In some embodiments, the alkylglycoside has an alkyl chain including between 8 to 20 carbons. In some embodiments, the alkylglycoside is selected from the group consisting of undecyl maltoside, dodecyl maltoside, tridecyl maltoside, tetradecyl maltoside, sucrose mono-dodecanoate, sucrose mono-tridecanoate, and sucrose mono-tetradecanoate. In some embodiments, the alkylglycoside is dodecyl-beta-D-maltoside. In some embodiments, the alkylglycoside concentration is between about 0.001% and 10.0% (w/v). In some embodiments, the alkylglycoside concentration is between about 0.05% and 0.5% (w/v).

In some embodiments, the composition further comprises a membrane penetration-enhancing agent. In some embodiments, the membrane penetration-enhancing agent is a surfactant, a bile salt, a phospholipid, an alcohol, an enamine, a medium and/or long-chain amphipathic molecules, a small hydrophobic molecule, sodium or a salicylic acid derivative, a glycerol ester of acetoacetic acid, a cyclodextrin, a medium-chain or long chain fatty acids, a chelating agent, an amino acid or salt thereof, an enzyme or combination thereof. In some embodiments, the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, sodium hydroxide, and combinations thereof. In some embodiments, the membrane penetration-enhancing agent is benzalkonium chloride, EDTA, or a combination thereof.

In some embodiments, the composition provides a Cmax for the epinephrine in the subject that is about 2 fold or greater as compared to administration without alkylglycoside.

In some embodiments, the composition provides a Tmax for the epinephrine in the subject that is about 2 fold or less as compared to administration without alkylglycoside.

In some embodiments, the composition provides a Tmax for the epinephrine of about 0.3 hours or less in the subject.

In some embodiments, the composition has a pH of about 2.0 to 6.0. In some embodiments, the composition has a pH of about 2.0 to 5.5. In some embodiments, the composition has a pH of about 2.0 to 5.0. In some embodiments, the composition has a pH of about 3.0 to 5.0. In some embodiments, the composition has a pH of about 3.0 to 5.5.

In some embodiments, compositions described here are liquid compositions suitable for intranasal administration.

In one aspect, the invention provides a method of increasing absorption of epinephrine into the circulatory system of a subject by administering, via the nasal, inhalation or pulmonary delivery route a composition comprising: (a) epinephrine; (b) an absorption increasing amount of a suitable nontoxic, nonionic alkylglycoside having a hydrophobic alkyl group joined by a linkage to a hydrophilic saccharide; and (c) a mucosal delivery-enhancing agent.

The term, “mucosal delivery-enhancing agent” includes agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a compound(s) (e.g., biologically active compound). Enhancement of mucosal delivery can occur by any of a variety of mechanisms, including, for example, by increasing the diffusion, transport, persistence or stability of the compound, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junction physiology, reducing the viscosity of mucus overlying the mucosal epithelium, reducing mucociliary clearance rates, and other mechanisms.

Exemplary mucosal delivery enhancing agents include the following agents and any combinations thereof:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from:
   (i) a surfactant;
   (ii) a bile salt;
   (ii) a phospholipid additive, mixed micelle, liposome, or carrier;
   (iii) an alcohol;
   (iv) an enamine;
   (v) an NO donor compound;
   (vi) a long-chain amphipathic molecule;
   (vii) a small hydrophobic penetration enhancer;
   (viii) sodium or a salicylic acid derivative;
   (ix) a glycerol ester of acetoacetic acid;
   (x) a cyclodextrin or beta-cyclodextrin derivative;
   (xi) a medium-chain fatty acid;
   (xii) a chelating agent;
   (xiii) an amino acid or salt thereof;
   (xiv) an N-acetylamino acid or salt thereof;
   (xv) an enzyme degradative to a selected membrane component;
   (ix) an inhibitor of fatty acid synthesis;
   (x) an inhibitor of cholesterol synthesis; and
   (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, mucoadhesive, support or complex-forming species with which the compound is effectively combined, associated, contained, encapsulate d or bound resulting in stabilization of the compound for enhanced nasal mucosal delivery, wherein the formulation of the compound with the intranasal delivery-enhancing agents provides for increased bioavailability of the compound in a blood plasma of a subject.

Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. For example, EDTA or its salts (e.g., sodium or potassium) are employed in amounts ranging from about 0.01% to 2% by weight of the composition containing alkylsaccharide preservative.

In yet another aspect, described herein is a pharmaceutical composition having a suitable nontoxic, nonionic alkylglycoside having a hydrophobic alkyl group joined by a linkage to a hydrophilic saccharide in combination with a mucosal delivery-enhancing agent selected from:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from:
   (i) a surfactant;
   (ii) a bile salt;
   (ii) a phospholipid additive, mixed micelle, liposome, or carrier;
   (iii) an alcohol;
   (iv) an enamine;
   (v) an NO donor compound;
   (vi) a long-chain amphipathic molecule;
   (vii) a small hydrophobic penetration enhancer;
   (viii) sodium or a salicylic acid derivative;
   (ix) a glycerol ester of acetoacetic acid;
   (x) a cyclodextrin or beta-cyclodextrin derivative;
   (xi) a medium-chain or long chain fatty acid;
   (xii) a chelating agent;
   (xiii) an amino acid or salt thereof;
   (xiv) an N-acetylamino acid or salt thereof;
   (xv) an enzyme degradative to a selected membrane component;
   (ix) an inhibitor of fatty acid synthesis;
   (x) an inhibitor of cholesterol synthesis; and
   (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

In another embodiment, described herein is a method of administering an alkylglycoside composition by administering a therapeutically effective amount of at least one alkylglycoside having an alkyl chain length from about 12 to about 14 carbon atoms, at least one saccharide with an antibacterial activity, and epinephrine.

In one aspect, provided herein is an antibacterial alkylsaccharide composition, which includes n-dodecyl-4-O-α-D-glucopyranosyl-β-D-glucopyranoside or n-tetradecyl-4-O-α-D-glucopyranosyl-β-D-glucopyranoside.

Accordingly, provided herein is Embodiment A1, a nasal spray formulation comprising between about 0.40 mg and about 2.40 mg per dose dispensed from the device of epinephrine, or a salt thereof. Alternative Embodiment A1, a nasal spray formulation comprising between about 0.40 mg and about 2.0 mg per dose dispensed from the device of epinephrine, or a salt thereof.

Embodiment A2. The nasal spray formulation as recited in Embodiment A1, wherein the formulation is a pharmaceutical formulation.

Embodiment A3. The nasal spray formulation as recited in Embodiment A2, wherein the epinephrine or salt thereof is present in the pharmaceutical formulation in an amount efficacious for the treatment of an acute hypersensitivity reaction.

Embodiment A4. The nasal spray formulation as recited in any of Embodiments A1-A3, wherein the formulation is aqueous.

Embodiment A5 The nasal spray formulation as recited in any of Embodiments A1 to A4, wherein the formulation comprises an absorption enhancer. In alternative Embodiment A5, the nasal spray formulation as recited in any of Embodiments A1-A4, wherein the formulation comprises one or more absorption enhancers.

Embodiment A6. The nasal spray formulation as recited in any of Embodiments A1-A5, wherein the formulation has intramuscular (IM)-injection-like or subcutaneous (SQ)-like absorption, or in between.

Embodiment A7. The nasal spray formulation as recited in Embodiment A6, wherein has intramuscular (IM)-injection-like absorption.

Embodiment A8. The nasal spray formulation as recited in Embodiment A6, wherein the formulation has subcutaneous (SC)-like absorption.

Embodiment A9. The nasal spray formulation as recited in Embodiment A8 where the SC pharmacokinetic profile has a Cₘₐₓ of at least 100 μg/mL and AUC₀₋₂₄₀ₘᵢₙ of 150 h*pg/mL.

Embodiment A10. The nasal spray formulation as recited in any of Embodiments A1-A9, wherein the formulation, when administered to a subject, yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.3 mg intramuscular injection yields;
a mean Cₘₐₓ that is at least 80% of the Cₘₐₓ and no more than 150% of the Cₘₐₓ that a 0.3 mg intramuscular injection yields;
a mean tₘₐₓ of less than 45 minutes; and
IM-injection like absorption under optimal dosing conditions in the thigh.

Embodiment A11. The nasal spray formulation as recited in any of Embodiments A1-A9, wherein the formulation, when administered to a subject, yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.15 mg intramuscular injection yields;
a mean Cₘₐₓ that is at least 80% of the Cₘₐₓ and no more than 150% of the Cₘₐₓ that a 0.15 mg intramuscular injection yields;
a mean tmax of less than 45 minutes; and
IM-injection like absorption under optimal dosing conditions in the thigh.

Embodiment A12. The nasal spray formulation as recited in any of Embodiments A1-A9, wherein the formulation, when administered to a subject, yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.5 mg intramuscular injection yields;
a mean Cₘₐₓ that is at least 80% of the Cmax and no more than 150% of the Cmax that a 0.5 mg intramuscular injection yields;
a mean tmax of less than 45 minutes; and
IM-injection like absorption under optimal dosing conditions in the thigh.

Embodiment A13. The nasal spray formulation as recited in Embodiment A1-A12, wherein the formulation, when administered to a subject, yields a tₘₐₓ of less than 40 minutes, a tₘₐₓ of less than 35 minutes, a tₘₐₓ of between 30 and 45 minutes, a tₘₐₓ of between 30 and 40 minutes, or a tₘₐₓ of between 30 and 35 minutes. Alternative Embodiment A13. The nasal spray formulation as recited in Embodiment A1-A12, wherein the formulation, when administered to a subject, yields a tₘₐₓ of less than 40 minutes, a tₘₐₓ of less than 35 minutes, a tₘₐₓ of between 15 and 45 minutes, a tₘₐₓ of between 20 and 45 minutes, a tₘₐₓ of between 25 and 45 minutes, a tₘₐₓ of between 30 and 45 minutes, a tₘₐₓ of between 30 and 40 minutes, a tₘₐₓ of between 30 and 35 minutes, a tₘₐₓ of between 15 and 20 minutes, a tₘₐₓ of between 15 and 25 minutes, or a tₘₐₓ of between 15 and 30 minutes.

Embodiment A14. The nasal spray formulation as recited in any of Embodiments A1-A13, wherein the formulation comprises less than one molar equivalents of acid to each mole of epinephrine.

Embodiment A15. The nasal spray formulation as recited in any of Embodiments A1-A13, wherein the formulation comprises between about 0.5 and about 1.1 molar equivalents of acid to each mole of epinephrine.

Embodiment A16. The nasal spray formulation as recited in either of Embodiments A14 and A15, wherein the acid is hydrochloric acid. Alternative Embodiment A16, The nasal spray formulation as recited in either of Embodiments A14 and A15, wherein the acid is acetic acid, adipic acid, ammonium chloride, boric acid, citric acid, hydrochloric acid, lactic acid, phosphoric acid, propionic acid, sulfuric acid, or tartaric acid.

Embodiment A17. The nasal spray formulation as recited in any of Embodiments A1-A16, wherein the formulation has a pH between about 3.0 and about 6.0. Alternative Embodiment A17. The nasal spray formulation as recited in any of Embodiments A1-A16, wherein the formulation has a pH between about 2.0 and about 6.0.

Embodiment A18. The nasal spray formulation as recited in Embodiment A17, wherein the formulation has a pH between about 3.5 and about 5.0.

Embodiment A19. The nasal spray formulation as recited in Embodiment A17, wherein the formulation has a pH between about 4.0 and about 4.5.

Embodiment A20. The nasal spray formulation as recited in Embodiment A17, wherein the formulation has a pH of about 4.5.

Embodiment A21. The nasal spray formulation as recited in Embodiment A17, wherein the formulation has a pH of about 4.0.

Embodiment A22. The nasal spray formulation as recited in any of Embodiments A1-A21, wherein the formulation comprises between about 5 mg/mL and about 40 mg/mL epinephrine, or a salt thereof. Alternative Embodiment A22. The nasal spray formulation as recited in any of Embodiments A1-A21, wherein the formulation comprises between about 3 mg/mL and about 40 mg/mL epinephrine, or a salt thereof.

Embodiment A23. The nasal spray formulation as recited in any of Embodiments A1-A22, wherein the formulation comprises between about 0.9 mg and about 2.4 mg per dose dispensed from the device of epinephrine, or a salt thereof.

Embodiment A24. The nasal spray formulation as recited in any of Embodiments A1-A22, wherein the formulation comprises between about 0.5 mg and about 2.0 mg per dose dispensed from the device of epinephrine, or a salt thereof.

Embodiment A25. The nasal spray formulation as recited in any of Embodiments A1-A22, wherein, the formulation comprises between about 0.75 mg and about 1.5 mg per dose dispensed from the device of epinephrine, or a salt thereof.

Embodiment A26. The nasal spray formulation as recited in any of Embodiments A1-A22, wherein the formulation comprises between about 0.9 mg and about 1.15 mg per dose dispensed from the device of epinephrine, or a salt thereof.

Embodiment A27. The nasal spray formulation as recited in any of Embodiments A1-A22, wherein the formulation comprises about 1.0 mg per dose dispensed from the device of epinephrine, or a salt thereof.

Embodiment A28. The nasal spray formulation as recited in any of Embodiments A1-A22, wherein the formulation comprises between about 0.45 mg and about 1.15 mg per dose dispensed from the device of epinephrine, or a salt thereof.

Embodiment A29. The nasal spray formulation as recited in any of Embodiments A1-A22, wherein the formulation comprises between about 1.0 mg and about 2.0 mg per dose dispensed from the device of epinephrine, or a salt thereof.

Embodiment A30. The nasal spray formulation as recited in any of Embodiments A1-A29, wherein the formulation additionally comprises a stabilizing agent.

Embodiment A31. The nasal spray formulation as recited in Embodiment A30, wherein the stabilizing agent is ethylenediaminetetraacetic acid (EDTA) or a salt thereof.

Embodiment A32. The nasal spray formulation as recited in Embodiment A31, wherein the EDTA is disodium EDTA.

Embodiment A33. The nasal spray formulation as recited in Embodiment A31 or A32, wherein the EDTA is present in an amount that is from 5% to 15% of the amount of the epinephrine, both measured in mmol. Alternative Embodiment A33. The nasal spray formulation as recited in Embodiment A31 or A32, wherein the EDTA is present in an amount that is from 0.001% (w/v) to 1% (w/v).

Embodiment A34. The nasal spray formulation as recited in Embodiment A31 or A32, wherein the mmol of EDTA is about 10% of the mmol of the epinephrine.

Embodiment A35. The nasal spray formulation as recited in any of Embodiments A1-A34, wherein the formulation additionally comprises a preservative.

Embodiment A36. The nasal spray formulation as recited in Embodiment A35, wherein the preservative is benzalkonium chloride.

Embodiment A37. The nasal spray formulation as recited in any of Embodiments A1-A34, wherein the formulation additionally comprises an absorption enhancer.

Embodiment A38. The nasal spray formulation as recited in Embodiment A37, wherein the absorption enhancer is an alkylsaccharide.

Embodiment A39. The nasal spray formulation as recited in Embodiment A38, wherein the absorption enhancer is dodecyl maltoside.

Embodiment A40. The nasal spray formulation as recited in Embodiment A39, wherein the formulation comprises about 0.005% (w/v) to about 2.5% (w/v) dodecyl maltoside.

Embodiment A41. The nasal spray formulation as recited in Embodiment A40, wherein the formulation comprises about 0.1% (w/v) to about 0.5% (w/v) dodecyl maltoside.

Embodiment A42. The nasal spray formulation as recited in Embodiment A41, wherein the formulation comprises about 0.25% (w/v) dodecyl maltoside. Alternative Embodiment A42. The nasal spray formulation as recited in Embodiment A41, wherein the formulation comprises about 0.25% (w/v) dodecyl maltoside and about 0.001 (w/v) to about 1% (w/v) benzalkonium chloride. Alternative Embodiment A42. The nasal spray formulation as recited in Embodiment A41, wherein the formulation comprises about 0.25% (w/v) dodecyl maltoside and about 0.001 (w/v) to about 1% (w/v) Oleic acid, or salt thereof. Alternative Embodiment A42. The nasal spray formulation as recited in Embodiment A41, wherein the formulation comprises about 0.25% (w/v) dodecyl maltoside, about 0.001 to about 1% (w/v) benzalkonium chloride and about 0.001 to about 1% (w/v) oleic acid, or salt thereof.

Embodiment A43a. In certain embodiments, the formulation comprises between about 0.75 mg and about 1.5 mg per dose dispensed from the device of epinephrine, or a salt thereof, and when administered as a nasal spray to a subject yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.3 mg intramuscular injection yields;
a mean Cₘₐₓ that is at least 80% of the Cₘₐₓ and no more than 150% of the Cₘₐₓ that a 0.3 mg intramuscular injection yields;
a mean tmax of less than 45 minutes; and
IM-injection like absorption under optimal dosing conditions in the thigh.

Embodiment A43b. In certain embodiments, the formulation comprises between about 0.5 mg and about 1.15 mg per dose dispensed from the device of epinephrine, or a salt thereof, and when administered as a nasal spray to a subject yields one or more of the following pharmacokinetic features:
Both the mean AUC₀₋₂₀ₘᵢₙ and AUC₀₋ₜ is at least 80% of the AUC₀₋₂₀ₘᵢₙ and AUC₀₋ₜ that a 0.15 mg intramuscular injection yields;
A mean Cₘₐ that is at least 80% of the Cₘₐₓ and no more than 150% of the Cₘₐₓ that a 0.15 mg intramuscular injection yields;
A mean tmax of less than 45 minutes
IM-injection like absorption under optimal dosing conditions in the thigh.

Embodiment A43c. In certain embodiments, the formulation comprises between about 1.0 mg and about 2.0 mg per dose dispensed from the device of epinephrine, or a salt thereof, and when administered as a nasal spray to a subject yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.5 mg intramuscular injection yields;
a mean Cₘₐₓ that is at least 80% of the Cmax and no more than 150% of the Cmax that a 0.5 mg intramuscular injection yields;
a mean tmax of less than 45 minutes; and
IM-injection like absorption under optimal dosing conditions in the thigh.

Also provided are embodiments wherein any of Embodiments A43a, A43b, and A43c comprise one or more of the limitations recited above in Embodiments A2-A22 and A30-A42.

Also provided herein is Embodiment A44, a nasal spray formulation comprising epinephrine, or a salt thereof, which when administered to a subject, yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.3 mg intramuscular injection yields;
a mean Cₘₐₓ that is at least 80% of the Cₘₐₓ and no more than 150% of the Cmax that a 0.3 mg intramuscular injection yields;
a mean tₘₐₓ of less than 45 minutes; and
IM-injection like absorption under optimal dosing conditions in the thigh.

Also provided herein is Embodiment A45, a nasal spray formulation comprising epinephrine, or a salt thereof, which when administered to a subject, yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.15 mg intramuscular injection yields;
a mean Cₘₐₓ that is at least 80% of the Cₘₐₓ and no more than 150% of the Cₘₐₓ that a 0.15 mg intramuscular injection yields;
a mean tₘₐₓ of less than 45 minutes; and
IM-injection like absorption under optimal dosing conditions in the thigh,

Also provided herein is Embodiment A46, a nasal spray formulation comprising epinephrine, or a salt thereof, which when administered to a subject, yields one or more of the following pharmacokinetic features:
both the mean AUC₀₋₂₀ₘᵢₙ and AUC₀₋ₜ are at least 80% of the AUC₀₋₂₀ₘᵢₙ and AUC₀₋ₜ that a 0.5 mg intramuscular injection yields;
a mean Cₘₐₓ that is at least 80% of the Cₘₐₓ and no more than 150% of the Cₘₐₓ that a 0.5 mg intramuscular injection yields;
a mean tₘₐₓ of less than 45 minutes; and
IM-injection like absorption under optimal dosing conditions in the thigh.

Embodiment A47. The nasal spray formulation as recited in any of Embodiments A45-A46, comprising between about 0.4 mg and about 2.40 mg per dose dispensed from the device of epinephrine, or a salt thereof.

Embodiment A48. The nasal spray formulation as recited in Embodiment A47, wherein the formulation is a pharmaceutical formulation.

Embodiment A49. The nasal spray formulation as recited in Embodiment A48, wherein the epinephrine or salt thereof is present in the pharmaceutical formulation in an amount efficacious for the treatment of an acute hypersensitivity reaction.

Embodiment A50. The nasal spray formulation as recited in any of Embodiments A44-A49, wherein the formulation is aqueous.

Embodiment A51. The nasal spray formulation as recited in any of Embodiments A44-A50, wherein the formulation has intramuscular (IM)-injection-like or subcutaneous (SQ)-like absorption.

Embodiment A52. The nasal spray formulation as recited in Embodiment A51, wherein the formulation has intramuscular (IM)-injection-like absorption.

Embodiment A53. The nasal spray formulation as recited in Embodiment A51, wherein the formulation has subcutaneous (SQ)-like absorption.

Embodiment A54. The nasal spray formulation as recited in Embodiment A1-A51 where the SC pharmacokinetic profile has a Cₘₐₓ of at least 100 μg/mL and AUC₀₋₂₄₀ₘᵢₙ of 150 h*pg/mL.

Embodiment A55. The nasal spray formulation as recited in any of Embodiments A44-A54, wherein the formulation comprises between about 5 mg/mL and about 40 mg/mL of epinephrine, or a salt thereof.

Embodiment A56. The nasal spray formulation as recited in any of Embodiments A44-A55, wherein the formulation comprises between about 0.9 mg and about 2.4 mg per dose dispensed from the device of epinephrine, or a salt thereof.

Embodiment A57. The nasal spray formulation as recited in any of Embodiments A44-A55, wherein the formulation comprises between about 0.5 mg and about 2.0 mg per dose dispensed from the device of epinephrine, or a salt thereof.

Embodiment A58. The nasal spray formulation as recited in any of Embodiments A44-A55, wherein, the formulation comprises between about 0.75 mg and about 1.5 mg per dose dispensed from the device of epinephrine, or a salt thereof.

Embodiment A59. The nasal spray formulation as recited in any of Embodiments A44-A55, wherein the formulation comprises between about 0.9 mg and about 1.15 mg per dose dispensed from the device of epinephrine, or a salt thereof.

Embodiment A60. The nasal spray formulation as recited in any of Embodiments A44-A55, wherein the formulation comprises about 1.0 mg per dose dispensed from the device of epinephrine, or a salt thereof.

Embodiment A61. The nasal spray formulation as recited in any of Embodiments A44-A55, wherein the formulation comprises between about 0.45 mg and about 1.15 mg per dose dispensed from the device of epinephrine, or a salt thereof.

Embodiment A62. The nasal spray formulation as recited in any of Embodiments A44-A55, wherein the formulation comprises between about 0.5 mg and about 2.0 mg per dose dispensed from the device of epinephrine, or a salt thereof.

Embodiment A63. The nasal spray formulation as recited in any of Embodiments A44-55, wherein the formulation comprises about 0.5 mg per dose dispensed from the device of epinephrine, or a salt thereof.

Embodiment A64. The nasal spray formulation as recited in any of Embodiments A44-A55, wherein the formulation comprises about 0.75 mg per dose dispensed from the device of epinephrine, or a salt thereof.

Embodiment A65. The nasal spray formulation as recited in any of Embodiments A44-A55, wherein the formulation comprises about 1.0 mg per dose dispensed from the device of epinephrine, or a salt thereof.

Embodiment A66. The nasal spray formulation as recited in any of Embodiments A44-A55, wherein the formulation comprises about 1.5 mg per dose dispensed from the device of epinephrine, or a salt thereof.

Embodiment A67. The nasal spray formulation as recited in any of Embodiments A44-A66, wherein the formulation comprises less than one molar equivalents of acid to each mole of epinephrine.

Embodiment A68. The nasal spray formulation as recited in any of Embodiments A44-A66, wherein the formulation comprises between about 0.5 and about 1.1 molar equivalents of acid to each mole of epinephrine.

Embodiment A69. The nasal spray formulation as recited in either of Embodiments A66 and A67, wherein the acid is a strong acid. Strong acids include hydrochloric acid, phosphoric acid, and sulfuric acid.

Embodiment A70. The nasal spray formulation as recited in Embodiment A69, wherein the acid is hydrochloric acid.

Embodiment A71. The nasal spray formulation as recited in any of Embodiments A44-A70, wherein no base is added to the formulation during its preparation.

Embodiment A72. The nasal spray formulation as recited in any of Embodiments A44-A71, wherein the formulation has a pH between about 3.0 and about 6.0. Alternative embodiment Embodiment A72. The nasal spray formulation as recited in any of Embodiments A44-A71, wherein the formulation has a pH between about 2.0 and about 6.0.

Embodiment A73. The nasal spray formulation as recited in Embodiment A72, wherein the formulation has a pH between about 3.5 and about 5.0.

Embodiment A74. The nasal spray formulation as recited in Embodiment A72, wherein the formulation has a pH between about 4.0 and about 4.5.

Embodiment A75. The nasal spray formulation as recited in Embodiment A72, wherein the formulation has a pH of about 4.5.

Embodiment A76. The nasal spray formulation as recited in Embodiment A72, wherein the formulation has a pH of about 4.0.

Embodiment A77. The nasal spray formulation as recited in any of Embodiments A44-A76, wherein the formulation additionally comprises a stabilizing agent.

Embodiment A78. The nasal spray formulation as recited in Embodiment A77, wherein the stabilizing agent is ethylenediaminetetraacetic acid (EDTA) or a salt thereof.

Embodiment A79. The nasal spray formulation as recited in Embodiment A78, wherein the EDTA is disodium EDTA.

Embodiment A80. The nasal spray formulation as recited in Embodiment A78, wherein the EDTA is present in an amount that is from 5% to 15% of the amount of the epinephrine, both measured in mmol. Alternative Embodiment A80. The nasal spray formulation as recited in Embodiment A78, wherein the EDTA is present in an amount that is from about 0.0010% (w/v) to about 1% (w/v).

Embodiment A81. The nasal spray formulation as recited in Embodiment A79, wherein the mmol of EDTA is about 10% of the mmol of the epinephrine.

Embodiment A82. The nasal spray formulation as recited in any of Embodiments A44-A81, wherein the formulation additionally comprises a preservative.

Embodiment A83. The nasal spray formulation as recited in Embodiment A82, wherein the preservative is benzalkonium chloride.

Embodiment A84. The nasal spray formulation as recited in any of Embodiments A44-A83, wherein the formulation additionally comprises an absorption enhancer. In alternative Embodiment A84, The nasal spray formulation as recited in any of Embodiments A44-A83, wherein the formulation additionally comprises one or more absorption enhancers.

Embodiment A85. The nasal spray formulation as recited in Embodiment A84, wherein the absorption enhancer is an alkylsaccharide. In alternative Embodiment A85, The nasal spray formulation as recited in Embodiment A84, wherein the absorption enhancer is an alkylsaccharide and/or benzalkonium chloride.

Embodiment A86. The nasal spray formulation as recited in Embodiment A85, wherein the absorption enhancer is dodecyl maltoside. In alternative Embodiment A86, The nasal spray formulation as recited in Embodiment A85, wherein the absorption enhancer is dodecyl maltoside, benzalkonium chloride, or a combination of dodecyl maltoside and benzylalkonium chloride.

Embodiment A87. The nasal spray formulation as recited in Embodiment A86, wherein the formulation comprises about 0.005% (w/v) to about 2.5% (w/v) dodecyl maltoside.

Embodiment A88. The nasal spray formulation as recited in Embodiment A87, wherein the formulation comprises about 0.1% (w/v) to about 0.5% (w/v) dodecyl maltoside.

Embodiment A89. The nasal spray formulation as recited in Embodiment A88, wherein the formulation comprises about 0.25% (w/v) dodecyl maltoside. Also provided is Embodiment A90, a method of treatment of a condition mediated by adrenergic receptors comprising the intranasal administration of the formulation as recited in any of Embodiments A1-A89 above.

Embodiment A91. The method as recited in Embodiment A90, wherein the condition is chosen from a type-1 hypersensitivity reaction (systemic allergic reaction), an acute asthmatic attack, cardiac arrest, and Stokes-Adams Syndrome.

Embodiment A92. The method as recited in Embodiment A91, wherein the condition is a type-1 hypersensitivity reaction (systemic allergic reaction).

Embodiment A93. The method as recited in Embodiment A92, wherein the type-1 hypersensitivity reaction (systemic allergic reaction) is chosen from allergic asthma, allergic conjunctivitis, allergic rhinitis, anaphylaxis, angioedema, urticaria, eosinophilia, drug allergy and food allergy.

Embodiment A94. The method as recited in Embodiment A93, wherein the drug allergy is an antibiotic allergy.

Embodiment A95. Also provided herein is a method of treatment of a systemic allergic reaction and anaphylaxis comprising the intranasal administration of an unbuffered intranasal formulation of epinephrine in an amount less than about 2.0 mg.

Also provided are embodiments wherein Embodiment A95 comprises one or more of the limitations recited above in Embodiments A2-A22, A25-A28, A30-A55, A58-A61, and A63-A89.

Embodiment A96. A pharmaceutical composition comprising: a) epinephrine; and b) an alkylglycoside; wherein the composition is formulated for administration into the circulatory system of a subject via the intranasal, inhalation, or pulmonary administration route. Alternative Embodiment A96. A pharmaceutical composition comprising: a) epinephrine; and b) an alkylglycoside; wherein the composition is a liquid formulated for intranasal delivery.

Embodiment A97. The pharmaceutical composition of Embodiment A96, wherein the alkylglycoside has an alkyl chain including between 8 to 20 carbons.

Embodiment A98. The pharmaceutical composition of Embodiment A97, wherein the alkylglycoside is selected from the group consisting of undecyl maltoside, dodecyl maltoside, tridecyl maltoside, tetradecyl maltoside, sucrose mono-dodecanoate, sucrose mono-tridecanoate, and sucrose mono-tetradecanoate.

Embodiment A99. The pharmaceutical composition of Embodiment A98, wherein the alkylglycoside is dodecyl-beta-D-maltoside.

Embodiment A100. The pharmaceutical composition of Embodiment A96, wherein the alkylglycoside concentration is between about 0.001% and 10.0% (w/v).

Embodiment A101. The pharmaceutical composition of Embodiment A100, wherein the alkylglycoside concentration is between about 0.05% and 0.5% (w/v).

Embodiment A102. The pharmaceutical composition of Embodiment A96, wherein the composition further comprises a membrane penetration-enhancing agent. The pharmaceutical composition of Embodiment A96, wherein the composition further comprises a membrane penetration-enhancing agent, pH modifier, buffering agents, isotonicity agent, antioxidant, chelator, preservative, or a combination thereof.

Embodiment A103. The pharmaceutical composition of Embodiment A102, wherein the membrane penetration-enhancing agent is a surfactant, a bile salt, a phospholipid, an alcohol, an enamine, a medium and/or long-chain amphipathic molecule, a small hydrophobic molecule, sodium or a salicylic acid derivative, a glycerol ester of acetoacetic acid, a cyclodextrin, a medium-chain or long chain fatty acid, a chelating agent, an amino acid or salt thereof, an enzyme or combination thereof.

Embodiment A104. The pharmaceutical composition of Embodiment A102, wherein the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, sodium hydroxide, and combinations thereof.

Embodiment A105. The pharmaceutical composition of Embodiment A102, wherein the membrane penetration-enhancing agent is benzalkonium chloride, EDTA, or a combination thereof.

Embodiment A106. The pharmaceutical composition of Embodiment A96, wherein the composition provides a Cₘₐₓ for the epinephrine in a subject that is about 2 fold or greater as compared to administration without alkylglycoside.

Embodiment A107. The pharmaceutical composition of Embodiment A96, wherein the composition provides a Tmax for the epinephrine in a subject that is about 2 fold or less as compared to administration without alkylglycoside.

Embodiment A108. The pharmaceutical composition of Embodiment A96, wherein the composition provides a Tmax for the epinephrine of about 0.3 hours or less in a subject.

Embodiment A109. The pharmaceutical composition of Embodiment A96, wherein the composition has a pH of about 2.0 to 5.0.

Embodiment A110. A method of increasing the bioavailability of epinephrine in a subject comprising administering to a subject a composition comprising epinephrine and an alkylglycoside, thereby increasing the bioavailability of the epinephrine in the subject, wherein the composition is administered into the circulatory system of the subject via the intranasal, inhalation, or pulmonary administration route. Alternative Embodiment A110. A method of increasing the bioavailability of epinephrine in a subject comprising administering to a subject a composition comprising epinephrine and an alkylglycoside, thereby increasing the bioavailability of the epinephrine in the subject, wherein the composition is a liquid composition administered intranasally.

Embodiment A111. The method of Embodiment A110, wherein the alkylglycoside has an alkyl chain including between 8 to 20 carbons.

Embodiment A112. The method of Embodiment A111, wherein the alkylglycoside is selected from the group consisting of undecyl maltoside, dodecyl maltoside, tridecyl maltoside, tetradecyl maltoside, sucrose mono-dodecanoate, sucrose mono-tridecanoate, and sucrose mono-tetradecanoate.

Embodiment A113. The method of Embodiment A112, wherein the alkylglycoside is dodecyl-beta-D-maltoside.

Embodiment A114. The method of Embodiment A110, wherein the alkylglycoside concentration is between about 0.001% and 10.0% (w/v).

Embodiment A115. The method of Embodiment A114, wherein the alkylglycoside concentration is between about 0.05% and 0.5% (w/v).

Embodiment A116. The method of Embodiment A110, wherein the composition further comprises a membrane penetration-enhancing agent.

Embodiment A117. The method of Embodiment A116, wherein the membrane penetration-enhancing agent is a surfactant, a bile salt, a phospholipid, an alcohol, an enamine, a long-chain amphipathic molecule, a small hydrophobic molecule, sodium or a salicylic acid derivative, a glycerol ester of acetoacetic acid, a cyclodextrin, a medium-chain fatty acid, a chelating agent, an amino acid or salt thereof, an enzyme or combination thereof.

Embodiment A118. The method of Embodiment A117, wherein the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, sodium hydroxide, and combinations thereof.

Embodiment A119. The method of Embodiment A116, wherein the membrane penetration-enhancing agent is benzalkonium chloride, EDTA, or a combination thereof.

Embodiment A120. The method of Embodiment A110, wherein the composition provides a Cmax for the epinephrine in the subject that is about 2 fold or greater as compared to administration without alkylglycoside.

Embodiment A121. The method of Embodiment A110, wherein the composition provides a Tmax for the epinephrine in the subject that is about 2 fold or less as compared to administration without alkylglycoside.

Embodiment A122. The method of Embodiment A110, wherein the composition provides a Tmax for the epinephrine of about 0.3 hours or less in the subject.

Embodiment A123. The method of Embodiment A110, wherein the composition has a pH of about 2.0 to 6.0. Alternative Embodiment A123. The method of Embodiment A110, wherein the composition has a pH of about 2.0 to 5.0.

Embodiment 1. A method of treating a type-1 hypersensitivity reaction in a human comprising intranasally administering to the human with the type-1 hypersensitivity reaction two or more doses of a pharmaceutical formulation of epinephrine, or a salt thereof; wherein each dose comprises between about 1.0 mg and about 2.0 mg of epinephrine, or a pharmaceutically acceptable salt thereof, and dodecyl maltoside; wherein the intranasal administration of two doses provides epinephrine pharmacokinetics in the human that is at least the same as the intramuscular injection of two 0.3 mg epinephrine doses in the anterolateral thigh; wherein the intranasal administration of each dose after the first dose provides dose proportional epinephrine pharmacokinetics; wherein each dose is intranasally administered in the same nostril; or wherein each dose is intranasally administered in opposite or alternating nostrils.

Embodiment 2. The method of Embodiment 1, wherein: each intranasally administered dose of the pharmaceutical formulation provides plasma epinephrine concentrations in the human that are efficacious for the elimination of, and/or for the prevention of, the further progression of, one, more than one, or all of the symptoms of the type-1 hypersensitivity reaction in the human; or the intranasal administration of the first dose of the pharmaceutical formulation to the human with the type-1 hypersensitivity reaction provides inadequate plasma epinephrine concentrations in the human that are efficacious for the elimination of, and/or for the prevention of, the further progression of, one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the elimination of, and/or for the prevention of the further progression of, one, more than one, or all of the symptoms of the type-1 hypersensitivity reaction in the human.

Embodiment 3. The method of Embodiments 1 or 2, wherein the human with the type-1 hypersensitivity reaction has symptoms of rhinitis; wherein the symptoms of rhinitis comprise nasal edema, congestion, or both.

Embodiment 4. The method of any one of Embodiments 1-3, wherein the type-1 hypersensitivity reaction comprises allergic asthma, allergic conjunctivitis, allergic rhinitis, anaphylaxis, angioedema, urticaria, eosinophilia, or combinations thereof.

Embodiment 5. The method of any one of Embodiments 1-3, wherein the type-1 hypersensitivity reaction comprises allergic asthma, allergic conjunctivitis, allergic rhinitis, anaphylaxis, angioedema, urticaria, eosinophilia, drug allergy or a food allergy.

Embodiment 6. The method of any one of Embodiments 1-3, wherein the type-1 hypersensitivity reaction comprises an antibiotic allergy

Embodiment 7. The method of any one of Embodiments 1-3, wherein the type-1 hypersensitivity reaction comprises anaphylaxis.

Embodiment 8. The method of any one of Embodiments 1-3, wherein the type-1 hypersensitivity reaction comprises an allergic reaction to an insect sting or bite, venom, allergen immunotherapy, foods, drugs, diagnostic testing substances or other allergens, or combinations thereof.

Embodiment 9. The method of any one of Embodiments 1-3, wherein the type-1 hypersensitivity reaction comprises idiopathic anaphylaxis or exercise-induced anaphylaxis.

Embodiment 10. The method of any one of Embodiments 1-3, wherein the type-1 hypersensitivity reaction comprises anaphylaxis; and the symptoms of anaphylaxis are selected from the group consisting of hives, generalized itching, nasal congestion, wheezing, difficulty breathing, cough, cyanosis, lightheadedness, dizziness, confusion, slurred speech, rapid pulse, palpitations, nausea or vomiting, abdominal pain or cramping, skin redness or skin inflammation, nasal flaring, and intercostal retractions.

Embodiment 11. The method of any one of Embodiments 1-3, wherein the type-1 hypersensitivity reaction comprises urticaria, and the symptoms of the type-1 hypersensitivity reaction comprise pruritis, flushing, or a burning sensation of the skin.

Embodiment 12. The method of any one of Embodiments 1-11, wherein intranasal administration of two doses provides pharmacokinetics that are greater than intramuscular injection of two 0.3 mg doses in the anterolateral thigh.

Embodiment 13. The method of any one of Embodiments 1-12, wherein the intranasal administration of the two doses provides plasma epinephrine concentrations with a Cₘₐₓ of at least 100 μg/mL, at least 200 μg/mL, or at least 300 μg/mL.

Embodiment 14. The method of any one of Embodiments 1-13, wherein the intranasal administration of the two doses provides plasma epinephrine concentrations with a time to maximum epinephrine plasma concentrations (Tmax) of less than 45 minutes, less than 35 minutes, less than 25 minutes, or less than 15 minutes.

Embodiment 15. The method of any one of Embodiments 1-14, wherein the pharmaceutical formulation is a nasal spray pharmaceutical formulation, and wherein the pharmaceutical formulation is a nasal spray pharmaceutical formulation comprising between about 1 mg/mL and about 40 mg/mL of epinephrine, or a salt thereof, in a volume between about 25 μL and about 250 μL per dose.

Embodiment 16. The method of any one of Embodiments 1-15, wherein each dose of the pharmaceutical formulation comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL, of epinephrine, or a salt thereof, in a volume between about 25 μL and about 250 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

Embodiment 17. The method of any one of Embodiments 1-15, wherein each dose of the pharmaceutical formulation comprises about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL of epinephrine, or a salt thereof, in a volume between about 50 μL and about 200 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

Embodiment 18. The method of any one of Embodiments 1-15, wherein each dose of the pharmaceutical formulation comprises about 5 mg/mL, about 6 mg/mL, about 7 mg/mL, about 8 mg/mL, about 9 mg/mL, or about 10 mg/mL of epinephrine, or a salt thereof, in a volume of about 200 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

Embodiment 19. The method of any one of Embodiments 1-15, wherein each dose of the pharmaceutical formulation comprises about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof, in a volume of about 100 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

Embodiment 20. The method of any one of Embodiments 1-15, wherein each dose of the pharmaceutical formulation comprises about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL of epinephrine, or a salt thereof, in a volume of about 50 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

Embodiment 21. The method of any one of Embodiments 1-15, wherein each dose of the pharmaceutical formulation comprises about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg of epinephrine, or a salt thereof, in a volume between about 50 μL and about 250 μL per dose; one or more other agents as excipients selected from the group consisting of isotonicity agents, stabilizing agents, antioxidants, preservatives, and pH adjustment agents to adjust the pH to a final pH between about 3.0 and about 5.5; and water.

Embodiment 22. The method of any one of Embodiments 16-21, wherein the isotonicity agents are selected from the group consisting of dextrose, glycerin, mannitol, potassium chloride, and sodium chloride; wherein the stabilizing agent is ethylenediaminetetraacetic acid (EDTA) or disodium salt thereof; wherein the antioxidants are selected from the group consisting of alpha tocopherol, D-α-tocopherol polyethylene glycol 1000 succinate, ascorbic acid, isoascorbic acid, butylated hydroxyanisole, citric acid monohydrate, potassium metabisulfite, sodium bisulfite, sodium metabisulfite, and sodium sulfite; wherein the preservative is benzalkonium chloride; and wherein the pH adjustment agents are selected from the group consisting of adipic acid, ammonium chloride, citric acid, acetic acid, hydrochloric acid, lactic acid, phosphoric acid, propionic acid, sulfuric acid, tartaric acid, sodium hydroxide, sodium citrate, sodium bicarbonate, and sodium carbonate.

Embodiment 23. The method of any one of Embodiments 16-21, wherein the isotonicity agents are selected from the group consisting of dextrose, glycerin, mannitol, potassium chloride, and sodium chloride; wherein the stabilizing agent is ethylenediaminetetraacetic acid (EDTA) or disodium salt thereof; wherein the antioxidants are selected from the group consisting of alpha tocopherol, ascorbic acid, isoascorbic acid, potassium metabisulfite, sodium bisulfite, and sodium metabisulfite, sodium sulfite; wherein the preservative is benzalkonium chloride; and wherein the pH adjustment agents are selected from the group consisting of hydrochloric acid and sodium hydroxide.

Embodiment 24. The method of any one of Embodiments 1-14, wherein each intranasally delivered dose of the pharmaceutical formulation comprises: about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof; dodecyl maltoside; sodium chloride; optionally sodium metabisulfite; optionally ethylenediaminetetraacetic acid (EDTA) or disodium salt thereof; optionally benzalkonium chloride; hydrochloric acid, sodium hydroxide, or both, in amounts to adjust the pH to a final pH between about 3.0 and about 5.5; and water; in a volume between about 100 μL and about 140 μL.

Embodiment 25. The method of any one of Embodiments 1-14, wherein each intranasally delivered dose of the pharmaceutical formulation comprises: about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof; about 2.0 mg/mL to about 3.0 mg/mL of dodecyl maltoside; about 5 to about 10 mg/mL of sodium chloride as an excipient; hydrochloric acid, sodium hydroxide, or both, in amounts to adjust the pH to a final pH between about 3.0 and about 5.5; optionally about 0.1 mg/mL to about 4.0 mg/mL of ethylenediaminetetraacetic acid (EDTA) or disodium salt thereof; optionally about 0.1 mg/mL to about 0.8 mg/mL of benzalkonium chloride as an excipient; optionally about 0.01 mg/mL to about 0.1 mg/mL of sodium metabisulfite as an excipient; and water; in a volume between about 100 μL and about 140 μL.

Embodiment 26. The method of any one of Embodiments 1-14, wherein each intranasally delivered dose of the pharmaceutical formulation comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; dodecyl maltoside; sodium chloride; optionally sodium metabisulfite; optionally ethylenediaminetetraacetic acid (EDTA) or disodium salt thereof; optionally benzalkonium chloride; hydrochloric acid, sodium hydroxide, or both, in amounts to adjust the pH to a final pH between about 3.0 and about 5.5; and water; in a volume between about 100 μL and about 140 μL.

Embodiment 27. The method of any one of Embodiments 1-14, wherein each intranasally delivered dose of the pharmaceutical formulation comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; about 2.0 mg/mL to about 3.0 mg/mL of dodecyl maltoside; about 5 to about 10 mg/mL of sodium chloride as an excipient; hydrochloric acid, sodium hydroxide, or both, in amounts to adjust the pH to a final pH between about 3.0 and about 5.5; optionally about 0.1 mg/mL to about 4.0 mg/mL of ethylenediaminetetraacetic acid (EDTA) or disodium salt thereof; optionally about 0.1 mg/mL to about 0.8 mg/mL of benzalkonium chloride as an excipient; optionally about 0.01 mg/mL to about 0.1 mg/mL of sodium metabisulfite as an excipient; and water; in a volume between about 100 μL and about 140 μL.

Embodiment 28. The method of any one of Embodiments 24-27, wherein: each dose of the nasal spray is intranasally administered with a nasal spray device; and one actuation of the nasal spray device delivers about 100 μL of the pharmaceutical formulation.

Embodiment 29. The method of any one of Embodiments 1-14, wherein each intranasally administered dose comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; dodecyl maltoside; sodium chloride; optionally sodium metabisulfite; optionally ethylenediaminetetraacetic acid (EDTA) or disodium salt thereof; optionally benzalkonium chloride; hydrochloric acid, sodium hydroxide, or both, in amounts to adjust the pH to a final pH between about 3.0 and about 5.5; and water; in a volume between about 100 μL.

Embodiment 30. The method of any one of Embodiments 1-14, wherein each intranasally administered dose comprises: about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof; about 2.0 mg/mL to about 3.0 mg/mL of dodecyl maltoside; about 5 to about 10 mg/mL of sodium chloride as an excipient; hydrochloric acid, sodium hydroxide, or both, in amounts to adjust the pH to a final pH between about 3.0 and about 5.5; optionally about 0.1 mg/mL to about 4.0 mg/mL of ethylenediaminetetraacetic acid (EDTA) or disodium salt thereof; optionally about 0.1 mg/mL to about 0.8 mg/mL of benzalkonium chloride as an excipient; optionally about 0.01 mg/mL to about 0.1 mg/mL of sodium metabisulfite as an excipient; and water; in a volume between about 100 μL.

Embodiment 31. A method of treating a type-1 hypersensitivity reaction in a human comprising intranasally administering to the human with the type-1 hypersensitivity reaction two or more doses of a pharmaceutical formulation of epinephrine, or a salt thereof; wherein epinephrine, or a pharmaceutically acceptable salt thereof, is the only pharmaceutically active ingredient in the nasal spray pharmaceutical formulation; wherein each dose comprises between about 0.1 mg and about 5.0 mg of epinephrine, or a pharmaceutically acceptable salt thereof.

Embodiment 32. The method of Embodiment 31, wherein the two or more doses of the pharmaceutical formulation are intranasally administered in the same nostril.

Embodiment 33. The method of Embodiment 31, wherein the each dose of the pharmaceutical formulation is intranasally administered in opposite or alternating nostrils.

Embodiment 34. A method of treating a type-1 hypersensitivity reaction in a human with an inadequate response to an intranasally administered dose of epinephrine, or a salt thereof, comprising intranasally administering to the human with the type-1 hypersensitivity reaction a second dose of a pharmaceutical formulation of epinephrine, or a salt thereof; wherein each dose comprises between about 0.1 mg and about 5.0 mg of epinephrine, or a pharmaceutically acceptable salt thereof; wherein epinephrine, or a pharmaceutically acceptable salt thereof, is the only pharmaceutically active ingredient in each dose of the nasal spray pharmaceutical formulation.

Embodiment 35. The method of Embodiment 34, wherein the second dose is intranasally administered in the same nostril as the first nostril that received the first dose.

Embodiment 36. The method of Embodiment 34, wherein the second dose is intranasally administered in the opposite nostril as the first nostril that received the first dose.

Embodiment 37. A method of enhancing the exposure to a second intranasally administered dose of epinephrine in a human in need of treatment for a type-1 hypersensitivity reaction comprising intranasally administering to the human with the type-1 hypersensitivity reaction two doses of a pharmaceutical formulation of epinephrine, or a salt thereof; wherein each dose comprises between about 0.1 mg and about 5.0 mg of epinephrine, or a pharmaceutically acceptable salt thereof; wherein epinephrine, or a pharmaceutically acceptable salt thereof, is the only pharmaceutically active ingredient in the nasal spray pharmaceutical formulation; wherein the second dose is intranasally administered in the same nostril as the first nostril.

Embodiment 38. The method of any one of Embodiments 31-37, wherein: the intranasal administration of each dose of the pharmaceutical formulation to the human with the type-1 hypersensitivity reaction provides plasma epinephrine concentrations in the human that are efficacious for the elimination of one, more than one, or all of the symptoms of the type-1 hypersensitivity reaction in the human.

Embodiment 39. The method of any one of Embodiments 31-37, wherein: the intranasal administration of the first dose of the pharmaceutical formulation to the human with the type-1 hypersensitivity reaction provides inadequate plasma epinephrine concentrations in the human that are efficacious for the elimination of one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the elimination of one, more than one, or all of the symptoms of the type-1 hypersensitivity reaction in the human.

Embodiment 40. The method of any one of Embodiments 31-39, wherein: the intranasal administration of each dose of the pharmaceutical formulation to the human with the type-1 hypersensitivity reaction provides plasma epinephrine concentrations in the human that are efficacious for the prevention of the further progression of one, more than one, or all of the symptoms of the type-1 hypersensitivity reaction in the human.

Embodiment 41. The method of any one of Embodiments 31-39, wherein: the intranasal administration of the first dose of the pharmaceutical formulation to the human with the type-1 hypersensitivity reaction provides inadequate plasma epinephrine concentrations in the human that are efficacious for the prevention of the further progression of one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the prevention of the further progression of one, more than one, or all of the symptoms of the type-1 hypersensitivity reaction in the human.

Embodiment 42. The method of any one of Embodiments 31-37, wherein: the intranasal administration of the first dose of the pharmaceutical formulation to the human with the type-1 hypersensitivity reaction provides plasma epinephrine concentrations in the human that are efficacious for the elimination of one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the prevention of the further progression of one, more than one, or all of the symptoms of the type-1 hypersensitivity reaction in the human.

Embodiment 43. The method of any one of Embodiments 31-42, wherein the human with the type-1 hypersensitivity reaction has symptoms of rhinitis; wherein the symptoms of rhinitis comprise nasal edema, congestion, or both.

Embodiment 44. The method of any one of Embodiments 31-43, wherein the type-1 hypersensitivity reaction comprises allergic asthma, allergic conjunctivitis, allergic rhinitis, anaphylaxis, angioedema, urticaria, eosinophilia, or combinations thereof.

Embodiment 45. The method of any one of Embodiments 31-43, wherein the type-1 hypersensitivity reaction comprises allergic asthma, allergic conjunctivitis, allergic rhinitis, anaphylaxis, angioedema, urticaria, eosinophilia, drug allergy or a food allergy.

Embodiment 46. The method of any one of Embodiments 31-43, wherein the type-1 hypersensitivity reaction comprises an antibiotic allergy

Embodiment 47. The method of any one of Embodiments 31-43, wherein the type-1 hypersensitivity reaction comprises anaphylaxis.

Embodiment 48. The method of any one of Embodiments 21-43, wherein the type-1 hypersensitivity reaction comprises an allergic reaction to: an insect sting or bite, venom, allergen immunotherapy, foods, drugs, diagnostic testing substances or other allergens, or combinations thereof.

Embodiment 49. The method of any one of Embodiments 31-43, wherein the type-1 hypersensitivity reaction comprises idiopathic anaphylaxis or exercise-induced anaphylaxis.

Embodiment 50. The method of any one of Embodiments 31-43, wherein the type-1 hypersensitivity reaction comprises anaphylaxis; and the symptoms of anaphylaxis are selected from the group consisting of hives, generalized itching, nasal congestion, wheezing, difficulty breathing, cough, cyanosis, lightheadedness, dizziness, confusion, slurred speech, rapid pulse, palpitations, nausea or vomiting, abdominal pain or cramping, skin redness or skin inflammation, nasal flaring, and intercostal retractions.

Embodiment 51. The method of any one of Embodiments 31-43, wherein the type-1 hypersensitivity reaction comprises urticaria, and the symptoms of the type-1 hypersensitivity reaction comprise pruritis, flushing, or a burning sensation of the skin.

Embodiment 52. The method of any one of Embodiments 31-51, wherein intranasal administration of the two doses provides pharmacokinetics that are at least the same as intramuscular injection of two 0.3 mg doses in the anterolateral thigh.

Embodiment 53. The method of any one of Embodiments 31-51, wherein intranasal administration of the two doses provides pharmacokinetics that are greater than intramuscular injection of two 0.3 mg doses in the anterolateral thigh.

Embodiment 54. The method of any one of Embodiments 31-53, wherein the intranasal administration of the two doses provides plasma epinephrine concentrations with a Cₘₐₓ of at least 100 pg/mL.

Embodiment 55. The method of any one of Embodiments 31-53, wherein the intranasal administration of the two doses provides plasma epinephrine concentrations with a Cₘₐₓ of at least 200 pg/mL.

Embodiment 56. The method of any one of Embodiments 31-53, wherein the intranasal administration of the two doses provides plasma epinephrine concentrations with a Cₘₐₓ of at least 300 pg/mL.

Embodiment 57. The method of any one of Embodiments 31-53, wherein the intranasal administration of the two doses provides plasma epinephrine concentrations with a time to maximum epinephrine plasma concentrations (Tmax) of less than 45 minutes.

Embodiment 58. The method of any one of Embodiments 31-57, wherein the intranasal administration of the two doses provides plasma epinephrine concentrations in the human that are efficacious for the treatment of the type-1 hypersensitivity reaction of with a time to maximum epinephrine plasma concentrations (Tmax) of less than 35 minutes.

Embodiment 59. The method of any one of Embodiments 31-57, wherein the intranasal administration of the two doses provides plasma epinephrine concentrations in the human that are efficacious for the treatment of the type-1 hypersensitivity reaction of with a time to maximum epinephrine plasma concentrations (Tmax) of less than 25 minutes.

Embodiment 60. The method of any one of Embodiments 31-57, wherein the intranasal administration of the two doses plasma epinephrine concentrations in the human that are efficacious for the treatment of the type-1 hypersensitivity reaction of with a time to maximum epinephrine plasma concentrations (Tmax) of less than 15 minutes.

Embodiment 61. The method of any one of Embodiments 31-60, wherein the pharmaceutical formulation is a nasal spray pharmaceutical formulation or a dry powder.

Embodiment 62. The method of any one of Embodiments 31-61, wherein the pharmaceutical formulation is a nasal spray pharmaceutical formulation comprising between about 1 mg/mL and about 40 mg/mL of epinephrine, or a salt thereof, in a volume between about 25 μL and about 250 μL per dose.

Embodiment 63. The method of any one of Embodiments 31-61, wherein each dose of the pharmaceutical formulation comprises between about 5 mg/mL and about 40 mg/mL of epinephrine, or a salt thereof.

Embodiment 64. The method of any one of Embodiments 31-61, wherein each dose of the pharmaceutical formulation comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL, of epinephrine, or a salt thereof.

Embodiment 65. The method of any one of Embodiments 31-61, wherein each dose of the pharmaceutical formulation comprises about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof.

Embodiment 66. The method of any one of Embodiments 31-61, wherein each dose of the pharmaceutical formulation comprises about 10 mg/mL of epinephrine, or a salt thereof, in a volume between about 50 μL and about 200 μL.

Embodiment 67. The method of any one of Embodiments 31-61, wherein each dose of the pharmaceutical formulation comprises about 20 mg/mL of epinephrine, or a salt thereof, in a volume between about 50 μL and about 200 μL.

Embodiment 68. The method of any one of Embodiments 31-67, wherein the pharmaceutical formulation further comprises one or more absorption enhancement agents as excipients.

Embodiment 69. The method of any one of Embodiments 31-67, wherein the pharmaceutical formulation further comprises one or more absorption enhancement agents as excipients, and optionally one or more other agents as excipients selected from the group consisting of isotonicity agents, stabilizing agents, antioxidants, preservatives, and pH adjustment agents to adjust the pH to a final pH between about 3.0 and about 5.0; and water.

Embodiment 70. The method of any one of Embodiments 31-67, wherein the pharmaceutical formulation further comprises one or more other agents as excipients selected from the group consisting of absorption enhancement agents, isotonicity agents, stabilizing agents, antioxidants, preservatives, and pH adjustment agents to adjust the pH to a final pH between about 3.0 and about 5.0; and water.

Embodiment 71. The method of any one of Embodiments 31-67, wherein: the absorption enhancement agents are selected from the group consisting of alkyl glycosides, fatty acids, bile salts, cyclodextrins, phospholipids, and alcohols; the isotonicity agents are selected from the group consisting of dextrose, glycerin, mannitol, potassium chloride, and sodium chloride; the stabilizing agent is ethylenediaminetetraacetic acid (EDTA) or disodium salt thereof; the antioxidants are selected from the group consisting of alpha tocopherol, D-α-tocopherol polyethylene glycol 1000 succinate, ascorbic acid, isoascorbic acid, butylated hydroxyanisole, citric acid monohydrate, potassium metabisulfite, sodium bisulfite, sodium metabisulfite, and sodium sulfite; the preservative is benzalkonium chloride; and the pH adjustment agents are selected from the group consisting of adipic acid, ammonium chloride, citric acid, acetic acid, hydrochloric acid, lactic acid, phosphoric acid, propionic acid, sulfuric acid, tartaric acid, sodium hydroxide, sodium citrate, sodium bicarbonate, and sodium carbonate.

Embodiment 72. The method of any one of Embodiments 68-71, wherein: the absorption enhancement agents are selected from the group consisting of dodecyl maltoside, polysorbate 20, polysorbate 80, oleic acid, sodium lauryl sulfate, sodium glycocholate, sodium taurocholate, and sodium taurodihydrofusidate.

Embodiment 73. The method of any one of Embodiments 68-71, wherein: the absorption enhancement agents are selected from the group consisting of dodecyl maltoside, sodium glycocholate, sodium taurocholate, and sodium taurodihydrofusidate.

Embodiment 74. The method of any one of Embodiments 68-71, wherein:
the absorption enhancement agent is dodecyl maltoside.

Embodiment 75. The method of any one of Embodiments 31-74, wherein the salt of epinephrine is selected from the group consisting of epinephrine acetate, epinephrine hydrochloride, epinephrine tartrate, epinephrine bitartrate, epinephrine hydrogen tartrate, and epinephrine borate.

Embodiment 76. The method of any one of Embodiments 69-75, wherein: the one or more pH adjustment agents are selected from the group consisting of hydrochloric acid, sodium hydroxide, and combination thereof.

Embodiment 77. The method of any one of Embodiments 31-61, wherein each intranasally delivered dose of the pharmaceutical formulation comprises about 10 mg/mL or about 20 mg/mL of epinephrine, or a salt thereof, in a volume between about 50 μL and about 200 μL; dodecyl maltoside, sodium chloride, optionally sodium metabisulfite, optionally ethylenediaminetetraacetic acid (EDTA) or disodium salt thereof, and optionally benzalkonium chloride; hydrochloric acid, sodium hydroxide, or both, in amounts to adjust the pH to a final pH between about 3.0 and about 5.0; and water.

Embodiment 78. The method of any one of Embodiments 31-61, wherein each intranasally delivered dose of the pharmaceutical formulation comprises about 10 mg/mL to about 20 mg/mL of epinephrine, or a salt thereof, in a volume between about 50 μL and about 200 μL; dodecyl maltoside, sodium chloride; hydrochloric acid, sodium hydroxide, or both, in amounts to adjust the pH to a final pH between about 3.0 and about 5.0; and water.

Embodiment 79. The method of any one of Embodiments 31-61, wherein each intranasally delivered dose of the pharmaceutical formulation comprises about 10 mg/mL or about 20 mg/mL of epinephrine, or a salt thereof, in a volume between about 50 μL and about 200 μL; dodecyl maltoside, sodium chloride, optionally sodium metabisulfite, optionally ethylenediaminetetraacetic acid (EDTA) or disodium salt thereof, and optionally benzalkonium chloride; hydrochloric acid, sodium hydroxide, or both, in amounts to adjust the pH to a final pH between about 3.0 and about 5.0; and water.

Embodiment 80. The method of any one of Embodiments 31-79, wherein each intranasally delivered dose of the pharmaceutical formulation comprises about 20 mg/mL of epinephrine, or a salt thereof, in a volume of about 50 μL, about 75 μL, about 100 μL, about 125 μL, about 150 μL, about 175 μL, about 200 μL, or about 250 μL.

Embodiment 81. The method of any one of Embodiments 31-61, wherein each intranasally delivered dose of the pharmaceutical formulation comprises about 10 mg/mL to about 20 mg/mL of epinephrine, or a salt thereof, in a volume between about 100 μL and about 140 μL; dodecyl maltoside, sodium chloride; hydrochloric acid, sodium hydroxide, or both, in amounts to adjust the pH to a final pH between about 3.0 and about 5.0; and water.

Embodiment 82. The method of any one of Embodiments 31-61, wherein each intranasally delivered dose of the pharmaceutical formulation comprises about 10 mg/mL or about 20 mg/mL of epinephrine, or a salt thereof, in a volume between about 100 μL and about 140 μL; dodecyl maltoside, sodium chloride, optionally sodium metabisulfite, optionally ethylenediaminetetraacetic acid (EDTA) or disodium salt thereof, and optionally benzalkonium chloride; hydrochloric acid, sodium hydroxide, or both, in amounts to adjust the pH to a final pH between about 3.0 and about 5.0; and water.

Embodiment 83. The method of any one of Embodiments 31-61, wherein each intranasally delivered dose of the pharmaceutical formulation comprises: about 3 mg/mL, about 5 mg/mL, about 6.5 mg/mL, about 10 mg/mL, about 13 mg/mL, about 15 mg/mL, or about 20 mg/mL of epinephrine in a volume between about 100 μL and about 140 μL; about 2.5 mg/mL of dodecyl maltoside; hydrochloric acid, sodium hydroxide, or combination thereof, to adjust the pH to a final pH between 3.0 and about 5.0; and water.

Embodiment 84. The method of any one of Embodiments 62-82, wherein each intranasally delivered dose of the pharmaceutical formulation further comprises: optionally about 2.0 mg/mL of ethylenediaminetetraacetic acid (EDTA) or disodium salt thereof; optionally about 0.4 mg/mL of benzalkonium chloride as an excipient; about 8.23 mg/mL of sodium chloride as an excipient; optionally about 0.05 mg/mL of sodium metabisulfite as an excipient.

Embodiment 85. The method of any one of Embodiments 62-84, wherein: each dose of the nasal spray is intranasally administered with a nasal spray device; and one actuation of the nasal spray device delivers about 100 μL of the pharmaceutical formulation.

Embodiment 86. The method of any one of Embodiments 62-84, wherein: each dose of the nasal spray is intranasally administered with a single-dose nasal spray device; and one actuation of the nasal spray device delivers about 100 μL of the pharmaceutical formulation.

Embodiment 87. The method of any one of Embodiments 31-61, wherein the pharmaceutical formulation is a dry powder in the form of a solid, amorphous, mono-particulate powder comprising a mixture of:
(a) epinephrine, or a pharmaceutically acceptable salt thereof; and
(b) a pharmaceutically-acceptable carrier material.

Embodiment 88. The method of Embodiments 87, wherein: the carrier material comprises a maltodextrin with a dextrose equivalent (DE) that is above 15; wherein the powder comprises particles of a size whereby, upon intranasal administration of said powder, said powder is delivered to nasal mucosa.

Embodiment 89. The method of any one of Embodiments 57-88, wherein the carrier material further comprises a disaccharide, selected from the group consisting of maltitol, trehalose, sucralose, sucrose, isomalt, maltose and lactose.

Embodiment 90. The method of any one of Embodiments 87-89, wherein the disaccharide comprises lactose and/or trehalose.

Embodiment 91. The method of any one of Embodiments 87-90, wherein the carrier material comprises a combination of trehalose and maltodextrin 19DE.

Embodiment 92. The method of any one of Embodiments 87-91, wherein the ratio of disaccharide:maltodextrin by weight, based on the total weight of the composition, is in the range of about 10:1 to about 1:20.

Embodiment 93. The method of any one of Embodiments 87-91, wherein the ratio of disaccharide:maltodextrin by weight, based on the total weight of the composition, is in the range of about 2:1 to about 1:8.

Embodiment 94. The method of any one of Embodiments 87-93, wherein the composition further comprises a sucrose ester.

Embodiment 95. The method of any one of Embodiments 87-94, wherein the sucrose ester comprises sucrose monolaurate.

Embodiment 96. The method of any one of Embodiments 87-95, wherein each dose comprises between about 0.5 mg and about 3 mg of epinephrine.

Embodiment 97. The method of any one of Embodiments 87-96, wherein the particles of the amorphous powder have a size distribution with a D10 value above about 10 μm and a D90 value below about 500 μm or below about 100 μm.

Embodiment 98. The method of any one of Embodiments 87-97, wherein the particles of said powder have a D90 below about 100 μm.

Embodiment 99. The method of Embodiment 87, wherein the pharmaceutically-acceptable carrier material is lactose.

Embodiment 100. The method of Embodiment 99, wherein each dose comprises between about 0.5 mg and about 4 mg of epinephrine.

Embodiment 101. The method of Embodiment 99, wherein each dose comprises about 1.6 mg or about 3.2 mg.

### Definitions

As used herein, the following terms have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range of numbers between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%. In some embodiments, “about” would encompass the recited value plus or minus 20%. In some embodiments, “about” would encompass the recited value plus or minus 15%. In some embodiments, “about” would encompass the recited value plus or minus 10%. In some embodiments, “about” would encompass the recited value plus or minus 5%.

“Weight per volume” or “w/v” refers to the mass in grams of a dissolved solute divided by the volume in milliliters of the entire solution. Typically, weight by volume is expressed as a percentage.

The term “absorption enhancer,” as used herein, refers to a functional excipient included in formulations to improve the absorption of an active agent such as a pharmacologically active drug. This term usually refers to an agent whose function is to increase absorption by enhancing nasal mucous-membrane permeation, rather than increasing solubility. As such, such agents are sometimes called permeation enhancers or penetration enhancers. In particular, absorption enhancers described herein may improve paracellular transport (i.e., passage through intercellular spaces and tight junctions), transcellular transport (i.e., passive diffusion or active transport across cellular membranes), or transcytosis (i.e., cellular vesicular uptake). Ozsoy et al., Molecules 14:3754-79, 2009.

Examples of absorption enhancers include alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl camitine, sodium lauryl sulfate, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, or salt thereof, oleyl alcohol, palmitic acid, polysorbate 80, propylene glycol, polyoxyethylene alkyl ethers, polyoxylglycerides, pyrrolidone, quillaia saponin, salicylic acid, sodium salt, β-sitosterol β-D-glucoside, sucrose cocoate, taurocholic acid, taurodeoxycholic acid, taurodihydrofusidic acid, thymol, tricaprylin, triolein, and alkylsaccharides, and combinations thereof, including but not limited to dodecyl maltoside, dodecyl-β-D-maltoside, tetradecyl maltoside, tetradecyl-β-D-maltoside and sucrose dodecanoate. Alkylsaccharides (e.g., nonionic alkylsaccharide surfactants such as alkylglycosides and sucrose esters of fatty acids that consist of an aliphatic hydrocarbon chain coupled to a sugar moiety by a glycosidic or ester bond, respectively), cyclodextrins (cyclic oligosaccharides composed of six or more monosaccharide units with a central cavity, which form inclusion complexes with hydrophobic molecules and they have primarily been used to increase drug solubility and dissolution and to enhance low molecular weight drug absorption), chitosans (linear cationic polysaccharides produced from the deacetylation of chitin), and bile salts and their derivatives (such as sodium glycocholate, sodium taurocholate, and sodium taurodihydrofusidate) tend to be amongst the best-tolerated absorption enhancers. See, e.g., Aungst B J, AAPS Journal 14(1):10-8, 2011; and Maggio, ET, Excipients and Food Chem. 5(2):100-12, 2014. Due to their chemical properties, certain absorption enhancers can function as preservatives and/or cationic surfactants in certain circumstances, depending on concentration in the formulation and other factors.

Described herein are compositions comprising epinephrine and at least one absorption enhancer and/or preservative and/or surfactant wherein the at least one absorption enhancer and/or preservative and/or surfactant comprises at least one alkylglycoside and/or at least one saccharide alkyl ester.

As used herein, the term “alkylsaccharide” (also referred to herein as “alkylglycoside”) refers to a type of an absorption enhancer. As used herein, an alkylsaccharide refers to any sugar joined by a linkage to any hydrophobic alkyl, as is known in the art. Alkylsaccharides include, but are not limited to: alkylsaccharides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers. The hydrophobic alkyl can be chosen of any desired size, depending on the hydrophobicity desired and the hydrophilicity of the saccharide moiety. For example, one preferred range of alkyl chains is from about 9 to about 24 carbon atoms. An even more preferred range is from about 9 to about 16 or about 14 carbon atoms. Similarly, some preferred saccharides include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, etc. The alkyl chain of an alkylsaccharide is often linked to the saccharide via a glycosidic bond, and accordingly, alkylsaccharides are often interchangeably referred to as alkylglycosides.

Any “suitable” alkylglycoside means one that fulfills the characteristics contemplated herein, i.e., that the alkylglycoside be nontoxic and nonionic, and that it increases the absorption of a compound (e.g. epinephrine) when it is administered with the compound via the nasal delivery route.

As use herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. The saccharide can be chosen, for example, from any currently commercially available saccharide species or can be synthesized. Some examples of the many possible saccharides to use include glucose, maltose, maltotriose, maltotetraose, sucrose and trehalose. Preferable saccharides include maltose, sucrose and glucose.

In some embodiments, described herein are composition that include at least one alkylglycoside and/or saccharide alkyl ester and epinephrine, methods of administering and using the compositions via the nasal delivery route, and methods of ameliorating a disease state in a subject by administration of such compositions.

In some embodiments, described herein is a method of administering a composition having at least one alkylglycoside and/or saccharide alkyl ester admixed, mixed, or blended with epinephrine and administered or delivered to a subject, wherein the alkyl has from about 10 to 24, 10 to 20, 10 to 16, or 10 to 14 carbon atoms, wherein the at least one alkylglycoside and/or saccharide alkyl ester increases the stability and bioavailability of the therapeutic agent.

In some embodiments, alkylsaccharides contemplated have a hydrophobic alkyl group linked to a hydrophilic saccharide. The linkage between the hydrophobic alkyl group and the hydrophilic saccharide can include, among other possibilities, a glycosidic, thioglycosidic (Horton), amide (Carbohydrates as Organic Raw Materials, F. W. Lichtenthaler ed., VCH Publishers, New York, 1991), ureide (Austrian Pat. 386,414 (1988); Chem. Abstr. 110:137536p (1989); see Gruber, H. and Greber, G., “Reactive Sucrose Derivatives” in Carbohydrates as Organic Raw Materials, pp. 95-116) or ester linkage (Sugar Esters: Preparation and Application, J. C. Colbert ed., (Noyes Data Corp., New Jersey), (1974)). Further, preferred glycosides can include maltose, sucrose, and glucose linked by glycosidic linkage to an alkyl chain of about 9-16 carbon atoms, e.g., nonyl-, decyl-, dodecyl- and tetradecyl sucroside, glucoside, and maltoside. These compositions are amphipathic and nontoxic, because they degrade to an alcohol and an oligosaccharide.

The above examples are illustrative of the types of glycosides contemplated, but the list is not exhaustive. Derivatives of the above compounds which fit the criteria described herein are also contemplated when choosing an alkylsaccharide.

In some embodiments, membrane penetration-enhancing agents contemplated serve as anti-bacterial agents. An agent is an “anti-bacterial” agent or substance if the agent or its equivalent destroy bacteria, or suppress bacterial growth or reproduction.

The term “active ingredient” or “pharmaceutically active compound” is defined in the context of a “formulation” and is intended to mean a component of a pharmaceutical formulation that provides the primary pharmacological effect, as opposed to an “inactive ingredient” which would generally be recognized as providing no pharmaceutical benefit.

The term “actuation,” as used herein, refers to operation of the device such that the pharmaceutical formulation is delivered therefrom.

The term “antimicrobial preservative,” as used herein, refers to a pharmaceutically acceptable excipient with antimicrobial properties which is added to a pharmaceutical formulation to maintain microbiological stability. Antimicrobial preservatives include, but are not limited to, antibacterial agents, antifungal agents, antioxidants, and preservatives.

The term “AUC,” as used herein, refers to the area under the drug plasma concentration-time curve. The term “AUC₀₋ₜ,” as used herein, refers to the area under the drug plasma concentration-time curve from t=0 to the last measured or measurable concentration. The term “AUC_{0-∞},” or equivalently, “AUC_{0-inf},” as used herein, refers to the area under the drug plasma concentration-time curve extrapolated to infinity (∞).

As used here, the term “benzalkonium chloride” (“BZK”) refers to a member of the class of quaternary ammonium compounds having the following structure:

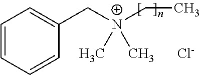

in which n is an integer. Benzalkonium chloride is a mixture of alkylbenzyl dimethylammonium chlorides, where a mixture of more than one n is used. In certain embodiments, n is 8, 10, 12, 14, 16, or 18. In other embodiments, n is 10, 12, or 14. In some embodiments, a mixture of n is 10, 12 and/or 14 predominate. In some embodiments, a mixture of n is 10, 12, 14 and/or 16 predominate. In some embodiments, benzalkonium chloride functions as a preservative (even in low amounts), an antiseptic, a disinfectant, a solubilizing and wetting agent, and/or a cationic surfactant. In some cases, benzalkonium chloride refers to a type of an absorption enhancer.

The term “bioavailability (F),” as used herein, refers to the fraction of a dose of drug that is absorbed from its site of administration and reaches, in an unchanged form, the systemic circulation. The term “absolute bioavailability” is used when the fraction of absorbed drug is related to its IV bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term “relative bioavailability (Fᵣₑₗ)” is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1}}$$

The term “clearance (CL),” as used herein, refers to the rate at which a drug is eliminated divided by its plasma concentration, giving a volume of plasma from which drug is completely removed per unit of time. CL is equal to the elimination rate constant (λ) multiplied by the volume of distribution (V_{d}) wherein “V_{d}” is the fluid volume that would be required to contain the amount of drug present in the body at the same concentration as in the plasma. The term “apparent clearance (CL/F),” as used herein, refers to clearance that does not take into account the bioavailability of the drug. It is the ratio of the dose over the AUC.

The term “Cₘₐₓ,” as used herein, refers to the maximum observed plasma concentration.

The term “coefficient of variation (CV),” as used herein, refers to the ratio of the sample standard deviation to the sample mean. It is often expressed as a percentage.

The term “confidence interval,” as used herein, refers to a range of values which will include the true average value of a parameter a specified percentage of the time.

The term “device,” as used herein, refers to an apparatus capable of delivering a drug to patient in need thereof.

The term “delivery time,” as used herein, refers to the amount of time that elapses between a determination made by a healthcare professional, or an untrained individual that an individual is in need of nasal delivery of epinephrine and completion of the delivery.

The term “disease,” as used herein, is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the “dose dispensed from the device” is typically measured in the nasal spray setting by the difference in weight of a device before and after actuation to release a dose of the formulation contained therein. The volume of liquid formulation and weight in milligrams of the active moiety contained therein may be determined by standard calculations.

The term “elimination rate constant (λ),” as used herein, refers to the fractional rate of drug removal from the body. This rate is constant in first-order kinetics and is independent of drug concentration in the body. λ is the slope of the plasma concentration-time line (on a logarithmic y scale). The term “λ_{∞}” as used herein, refers to the terminal phase elimination rate constant, wherein the “terminal phase” of the drug plasma concentration-time curve is a straight line when plotted on a semi-logarithmic graph. The terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

The term “equal,” as used herein, means essentially the same as (i.e., negligibly different from) in quantity, amount, value, degree, or size. The term “equal” may, in certain embodiments, include “bioequivalent,” but the terms are not coterminous.

The term “bioequivalent,” as used herein, describes the relationship between a reference and a putative equivalent or alternative drug, and per 21 C.F.R. § 320.1, means that there is no significant difference in the rate and extent to which the active ingredient or active moiety in pharmaceutical equivalents or pharmaceutical alternatives becomes available at the site of drug action when administered at the same molar dose under similar conditions in an appropriately designed study. Rate and extent of absorption may be determined from, or informed by, Cₘₐₓ and AUC, respectively. In certain embodiments, statistical criteria may be used, e.g., between 80% and 125% of a reference value, or 90% CI.

The term “molar equivalent,” as used herein, refers to an amount of epinephrine that is equimolar to a specified amount of acid.

The term “excipient,” as used herein, refers to a natural or synthetic substance formulated alongside the active ingredient of a medication. An excipient is included in a formulation for a variety of reasons such as, but not limited to, long-term stabilization, bulking up solid formulations, or to confer a therapeutic enhancement on the active ingredient in the final dosage form, such as facilitating drug absorption, reducing viscosity, or enhancing solubility.

The term “filled,” as used herein, refers to an association between a device and a pharmaceutical formulation, for example, when a pharmaceutical formulation described herein comprising a therapeutically effective amount of epinephrine is present within a reservoir that forms a part of a device described herein.

The term “formulation,” with or without the modifier “pharmaceutical,” as used herein, refers to a composition comprising at least one physiologically active ingredient (e.g., a drug); including but not limited to, salts, solvates and hydrates of epinephrine and related compounds described herein, whereby the formulation is amenable to use for a specified, efficacious outcome in a mammal (for example, without limitation, a human).

The term “pharmaceutical formulation,” as used herein, alone or in combination, refers to a formulation that is suited for use for treatment (or in certain embodiments, prevention) of a disease in a subject.

The term “hydrate,” as used herein, refers to epinephrine described herein or a salt thereof that further includes a stoichiometric or non-stoichiometric amount of water bound by non-covalent intermolecular forces.

The term “in need of treatment” and the term “in need thereof” when referring to treatment are used interchangeably and refer to a judgment made by a caregiver (e.g. physician, nurse, nurse practitioner, that a patient will benefit from treatment.

As used herein, an “intramuscular (IM) injection” of epinephrine is typically administered via an IM epinephrine delivered by auto injector in the thigh, e.g., in the vestus lateralis muscle (referred to herein as “optimal dosing conditions in the thigh”). As such, when comparing pharmacokinetic parameters yielded by IM epinephrine injection to those yielded by IN epinephrine administration, the comparison should be assumed to be as if the IM injection were in the thigh, which is the optimal dosing method for epinephrine. In one embodiment, IM epinephrine injection is achieved with EpiPen® Auto-Injector (0.3 mg/0.3 mL epinephrine injection, USP, pre-filled auto-injector; Mylan Specialty L.P.).

As used herein, an “subcutaneous (SQ) injection” of epinephrine is typically administered by injection into the subcutaneous layer of the deltoid region in the upper arm. Simons et al. Epinephrine absorption in adults: Intramuscular versus subcutaneous injection. J Allergy. Clin. Immunol. 2001; 108:871-3.

As used herein, two embodiments are “mutually exclusive” when one is defined to be something which is different than the other. For example, an embodiment wherein the concentration of epinephrine is specified to be 5 mg/mL is mutually exclusive with an embodiment wherein the amount of epinephrine is specified to be 10 mg/mL. However, an embodiment wherein the amount of epinephrine is specified to be 5 mg/mL is not mutually exclusive with an embodiment in which less than about 10% of the pharmaceutical formulation leaves the nasal cavity via drainage into the nasopharynx or externally.

The term “pharmaceutically acceptable,” as used herein, refers to a component of a formulation, often referred to as a carrier or excipient, that is compatible with the other ingredients of the formulation and not overly deleterious to the recipient thereof.

The term “pre-primed,” as used herein, refers to a device, such as a nasal spray which can deliver a formulation to a patient in need thereof with the first actuation of the spray pump, i.e., without the need to prime the pump prior to dosing, such as by actuating the pump one or more times until a spray appears.

The term “prone,” as used herein, refers to a patient who is lying face down.

As used herein, the term “protective packaging” refers to overwrap.

The term “solvate,” as used herein, refers to epinephrine described herein or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “storage-stable,” as used herein, refers to a formulation in which at least about 90% to 115% of the active ingredient remains within acceptable regulatory specifications after storage of the formulation at specified temperature and humidity for a specified time, for example, for at least 12 months at 25° C. and 60% relative humidity and about six months at about 40° C. and about 75% relative humidity.

The term “subject,” as used herein, is intended to be synonymous with “patient,” and refers to any mammal (preferably human) afflicted with a condition likely to benefit from treatment with a therapeutically effective amount epinephrine, e.g., a subject experiencing a type-1 hypersensitivity reaction (systemic allergic reaction) such as anaphylaxis.

The term “supine,” as used herein, refers to a patient who is lying face up.

The term “nostril,” as used herein, is synonymous with “naris.”

The term “therapeutically effective amount” or “therapeutically effective dose,” as used herein, refers to the amount or dose of active compound or pharmaceutical agent that elicits the biological or medicinal response in a tissue, system, or individual that is being sought by a researcher, healthcare provider or individual. In some embodiments, a therapeutically effective amount eliminates or reduces the severity of one, more, or all symptoms of a disease, disorder, or condition being treated. In some embodiments, a therapeutically effective amount prevents the further progression of one, more than one, or all of the symptoms of the disease, disorder, or condition being treated.

The term “treat” or “treating” with reference to a disease, disorder, or condition and the use of epinephrine refers to providing plasma epinephrine concentrations in the human that are efficacious for the elimination of one, more than one, or all of the symptoms of the disease, disorder, or condition in the human, or for the prevention of the further progression of one, more than one, or all of the symptoms of the disease, disorder, or condition in the human. In some embodiments, “treat” or “treating” with reference to a disease, disorder, or condition and the use of epinephrine refers to providing plasma epinephrine concentrations in the human that are efficacious for the elimination of one, more than one, or all of the symptoms of the disease, disorder, or condition in the human. In some embodiments, “treat” or “treating” with reference to a disease, disorder, or condition and the use of epinephrine refers to providing plasma epinephrine concentrations in the human that are efficacious for prevention of the further progression of one, more than one, or all of the symptoms of the disease, disorder, or condition in the human.

The term “t_{1/2}” or “half-life,” as used herein, refers to the amount of time required for half of a drug or other analyte of interest (for example, an adrenergic receptor agonist) to be eliminated from the body or the time required for a drug concentration to decline by half.

The term “tonicity agent,” as used herein, refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like. In some embodiments, formulations contemplated herein include one or more tonicity agents selected from dextrose, glycerin, mannitol, potassium chloride and sodium chloride. In some embodiments, formulations contemplated herein include sodium chloride as a tonicity agent.

The term “tomography,” as used herein, refers to a process of imaging by sections. The images may be looked at individually, as a series of two-dimensional slices or together, as a computer-generated three-dimensional representation.

The term “Tₘₐₓ,” as used herein, refers to the time, from administration, for a drug or other analyte to reach maximum drug plasma concentration (Cₘₐₓ).

### Epinephrine

The term “epinephrine” as used herein refers to the compound (R)-4-(1-Hydroxy-2-(methylamino)ethyl)benzene-1,2-diol, also known as adrenaline, shown below and having the following structure, elemental makeup, molecular weight, and CAS Registry Number:

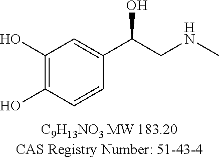

The term include any metabolite, salt, ester, hydrate, anhydride, solvate, isomer, isotope, enantiomer, free acid form, free base form, crystalline form, co-crystalline form, complexes, amorphous form, pro-drug (including ester pro-drug) form, racemate, polymorph, chelate, isomer, tautomer, or optically active form thereof, or mixture of any two or more of the foregoing.

Provided are drug products adapted for nasal delivery of epinephrine, including formulations and devices. Epinephrine acts by binding to a variety of adrenergic receptors. Epinephrine is a nonselective agonist of all adrenergic receptors, including the major subtypes α1, α2, β1, β2, and β3. Its actions vary by tissue type and tissue expression of adrenergic receptors. For example, high levels of epinephrine causes smooth muscle relaxation in the airways but causes contraction of the smooth muscle that lines most arterioles.

Provided are formulations, devices adapted for nasal delivery of a formulation to a patient, kits comprising the foregoing, and methods of using the same in treatment, each comprising a therapeutically effective amount of epinephrine.

Epinephrine may be present in the formulations administered herein at concentrations between 1 mg/mL and 40 mg/mL, for example, at concentrations of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL.

Epinephrine may be present in the formulations administered herein at doses between 0.1 mg and 4 mg, for example, at doses of about 0.5 mg, about 1.0 mg, or about 2.0 mg. These doses may be scaled based on molecular weight of a counterion if a salt is used to prepare the formulation.

Epinephrine may optionally exist as a pharmaceutically acceptable salt including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent. Due to the perceived insolubility of epinephrine base, finished dosage forms of epinephrine used in healthcare (solutions, aerosols, etc.) are typically salts, e.g. hydrochloride, bitartrate, or borate salts. In certain embodiments, formulations contemplated herein include a salt form of epinephrine that is epinephrine acetate, epinephrine hydrochloride, epinephrine tartrate, epinephrine bitartrate, epinephrine hydrogen tartrate or epinephrine borate.

Accordingly, provided herein are pharmaceutical formulations for intranasal administration comprising epinephrine. In certain embodiments, the formulation is an aqueous solution. In certain embodiments, the formulation comprises, per dose, between about 25 and about 250 μL of the aqueous solution. In certain embodiments, the formulation comprises, per dose, between about 50 and about 250 μL of the aqueous solution. In certain embodiments, the formulation comprises, per dose, between about 50 and about 200 μL of the aqueous solution. In certain embodiments, the formulation comprises, per dose, not more than about 140 μL. In certain embodiments, the formulation comprises, per dose, not more than about 100 μL. In certain embodiments, the formulation comprises, per dose, about 100 μL. The formulation may comprise, per dose, about 25 μL, about 50 μL, about 75 μL, about 100 μL, about 125 μL, about 150 μL, about 175 μL, about 200 μL, or about 250 μL of the aqueous solution.

The pharmaceutical formulations for intranasal administration comprising epinephrine described herein bypass potential metabolic conversion in the gastrointestinal tract and hepatic first-pass metabolism, and reach the systemic circulation in a pharmacologically active form. Epinephrine is extensively metabolized after oral administration by the catechol-O-methyltransferase in the gastrointestinal tract and by monoamine oxidase in the gastrointestinal tract and in the liver. Avoiding first pass clearance assures that more of the epinephrine that is administered will be available to treat anaphylaxis. By avoiding first pass liver clearance, the bioavailability of the epinephrine is increased.

### Formulations

Also provided are pharmaceutical formulations comprising epinephrine. Certain embodiments of the present disclosure include a method of producing a formulation comprising admixing epinephrine and a pharmaceutically acceptable carrier. Pharmaceutical formulations are applied directly to the nasal cavity using the devices described herein. In the case of a spray, this may be achieved for example by means of a metering atomizing spray pump, e.g. a single, bi-dose or multiuse spray device, with or without a propellant.

Liquid preparations include solutions, suspensions and emulsions, for example, water, or water-ethanol, or water-propylene glycol solutions. Typically, the formulation is an aqueous liquid solution. Additional ingredients in liquid preparations may include preservatives, stabilizing agents, tonicity agents, absorption enhancers, pH-adjusting agents, antioxidants, buffers, sweetners/flavoring agents/task-masking agents, and optionally other ingredients. Ingredients in liquid preparations may serve different functions. The function(s) of a particular ingredient will depend on a number of factors including, but not limited to, presence or absence of other ingredients, concentration(s), and other factors.

Preservatives include: benzalkonium chloride, methylparaben, sodium benzoate, benzoic acid, phenyl ethyl alcohol, and the like, and mixtures thereof. Due to their chemical properties, certain preservatives can function as a surfactants and/or absorption enhancers in certain circumstances, depending on concentration in the formulation and other factors.

Other preservatives include: alcohol, benzalkonium chloride, benzethonium chloride, benzoic acid, benzyl alcohol, boric acid, bronopol, butylated hydroxyanisole (BHA), butylene glycol, butylparaben, calcium acetate, calcium chloride, calcium lactate, carbon dioxide, bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, magnesium trisilicate, isopropyl alcohol, lactic acid, methylparaben, monothioglycerol, parabens (methyl, ethyl and propyl), pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, propylparaben, propylparaben sodium, sodium acetate, sodium benzoate, sodium borate, sodium lactate, sodium metabisulfite, sodium propionate, sodium sulfite, sorbic acid, sulfobutyletherb-cyclodextrin, sulfur dioxide, edetic acid, thimerosal, and xylitol.

In some embodiments, preservatives include, but are not limited to, antibacterial agents, antifungal agents, and antioxidants.

Antibacterial agents include, but are not limited to, chlorocresol, diazolidinyl urea, dimethyl sulfoxide, glacial acetic acid, imidurea, iodine/edetic acid, phenylmercuric acetate, phenylmercuric borate, phenylmercuric hydroxide, potassium sorbate, sodium hydroxide, sorbic acid, thymol, antiseptics, and disinfectants.

Antifungal agents include, but are not limited to, benzoic acid, butylene glycol, butylparaben, chlorocresol, coconut oil, dimethyl sulfoxide, ethylparaben, glacial acetic acid, imidurea, methylparabens, phenylmercuric acetate, phenylmercuric borate, phenylmercuric hydroxide, potassium sorbate, propylparaben, sodium propionate, sodium thiosulfate, thymol, and vanillin.

Surfactants include but are not limited to: Poly sorbate 80 NF, poly oxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof. Due to their chemical properties, certain surfactants can function as a preservatives and/or absorption enhancers in certain circumstances, depending on concentration in the formulation and other factors.

Surfactants include but are not limited to: cationic, anionic, nonionic and zwitterionic surfactants.

Surfactants also include: anionic surfactants (e.g. carboxylates sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols, ethoxylated and sulphated), nonionic surfactants (e.g. ethoxylated aliphatic alcohol, polyoxyethylene surfactants, carboxylic esters, polyethylene glycol esters, anhydrosorbitol ester and it's ethoxylated derivatives, glycol esters of fatty acids, carboxylic amides, monoalkanolamine condensates, polyoxyethylene fatty acid amides), cationic surfactants (e.g. quaternary ammonium salts, amines with amide linkages, polyoxyethylene alkyl and alicyclic amines, 4.n,n,n′,n′ tetrakis substituted ethylenediamines, 2-alkyl 1-hydroxethyl 2-imidazolines), amphoteric surfactants (amphoteric surfactants contains both an acidic and a basic hydrophilic moiety in their surface e.g., n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, sodium salt, etc.).

Antioxidants include, but are not limited to, tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, or salt thereof, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof.

Buffers include, but are not limited to, phosphate buffers, acetate buffers, and citrate buffers.

In some embodiments, the nasal spray formulation comprises a buffering agent. Buffering agents include, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

Isotonicity agents include sodium chloride, calcium chloride, magnesium chloride, sorbitol, sucrose, dextrose, lactose, mannitol, trehalose, raffinose, polyethylene glycol, hydroxyethyl starch, glycerine, glycine, and the like, and mixtures thereof. In certain embodiments, the isotonicity agent is chosen from dextrose, glycerin, mannitol, potassium chloride, and sodium chloride. In certain embodiments, the isotonicity agent is sodium chloride. In certain embodiments, the formulations disclosed herein contain sodium chloride in an amount sufficient to cause the final composition to have a nasally acceptable osmolality, preferably 240-350 mOsm/kg. In certain embodiments, the formulations contain 0.3-1.9% sodium chloride.

Sweetners/flavoring agents/task-masking agents include, but are not limited to, sucrose, dextrose, lactose, sucralose, acesulfame-K, aspartame, saccharin, sodium saccharin, citric acid, aspartic acid, eucalyptol, mannitol, glycerin, xylitol, menthol, glycyrrhizic acid, cinnamon oils, oil of wintergreen, peppermint oils, clover oil, bay oil, anise oil, eucalyptus, vanilla, citrus oil such as lemon oil, orange oil, grape and grapefruit oil, fruit essences including apple, peach, pear, strawberry, raspberry, cherry, plum, pineapple, apricot, etc. and combinations thereof. In some embodiments, the formulations contain from about 0.0001 percent to about 1 percent of a sweetener/flavoring agent/task-masking agent, and may be present at lower or higher amounts as a factor of one or more of potency of the effect on flavor, solubility of the flavorant, effects of the flavorant on solubility or other physicochemical or pharmacokinetic properties of other formulation components, or other factors.

In certain embodiments, the pharmaceutical formulation additionally comprises an isotonicity agent. The intranasal formulation may comprise between about 0.2% (w/v) and about 1.2% (w/v) isotonicity agent, such as about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.10% (w/v), or about 1.2% (w/v). The intranasal formulation may comprise more than about 0.10% (w/v) isotonicity agent. The intranasal formulation may comprise less than about 1.2% (w/v) isotonicity agent. In other embodiments, the intranasal formulation may comprise between about 0.2% (w/v) and about 1.9% (w/v) isotonicity agent, such as about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), or about 1.9% (w/v). The intranasal formulation may comprise less than about 1.9% (w/v) isotonicity agent.

In certain embodiments, the formulation additionally comprises an absorption enhancer. In certain embodiments, the pharmaceutical formulation comprises between about 0.005% (w/v) to about 2.5% (w/v) of the absorption enhancer. In certain embodiments, the pharmaceutical formulation comprises between about 0.05% (w/v) to about 2.5% (w/v) of the absorption enhancer. In certain embodiments, the pharmaceutical formulation comprises between about 0.10% (w/v) to about 0.5% (w/v) of the absorption enhancer. In certain embodiments, the pharmaceutical formulation comprises about 0.25% (w/v) of the absorption enhancer. In certain embodiments, the pharmaceutical formulation comprises about 0.18% (w/v) of the absorption enhancer.

In certain embodiments, the absorption enhancer is selected from benzalkonium chloride, cyclodextrins, chitosan, deoxycholic acid, an alkylsaccharide (e.g., a nonionic alkylsaccharide surfactant such as an alkylglycoside and a sucrose ester of fatty acids that consists of an aliphatic hydrocarbon chain coupled to a sugar moiety by a glycosidic or ester bond, respectively), fusidic acid derivatives, glycocholic acid, laureth-9, phosphatidylcholines, taurocholic acid, taurodihydrofusidic acid, microspheres and liposomes, and bile salts. In certain embodiments, the absorption enhancer is benzalkonium chloride. The formulation may comprise about 0.010% (w/v) to about 1% (w/v) benzalkonium chloride. In certain embodiments, the pharmaceutical formulation comprises about 0.005% (w/v) to about 0.015% (w/v) benzalkonium chloride. In certain embodiments, the pharmaceutical formulation comprises about 0.01% (w/v), about 0.02% (w/v), about 0.03% (w/v), or about 0.04% (w/v) of benzalkonium chloride. In certain embodiments, the pharmaceutical formulation comprises about 0.01% (w/v) benzalkonium chloride. In certain embodiments, the pharmaceutical formulation comprises about 0.02% (w/v) benzalkonium chloride. In certain embodiments, the pharmaceutical formulation comprises about 0.04% benzalkonium chloride.

In certain embodiments, the pharmaceutical formulation comprises benzalkonium chloride in an amount between about 0.001% (w/v) and about 1% (w/v). In certain other embodiments, the pharmaceutical formulation comprises benzalkonium chloride in an amount between about 0.001% (w/v) and about 0.5% (w/v). In certain other embodiments, the pharmaceutical formulation comprises benzalkonium chloride in an amount between about 0.001% (w/v) and about 0.2% (w/v). In some embodiments, the pharmaceutical formulation comprises 0.001% (w/v), 0.003% (w/v), 0.0050% (w/v), 0.007% (w/v), 0.009% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.2% (w/v), 0.31% (w/v), 0.22% (w/v), 0.23% (w/v), 0.24% (w/v), 0.25% (w/v), 0.26% (w/v), 0.27% (w/v), 0.28% (w/v), 0.29% (w/v), 0.3% (w/v), 0.31% (w/v), 0.32% (w/v), 0.33% (w/v), 0.34% (w/v), 0.35% (w/v), 0.36% (w/v), 0.37% (w/v), 0.38% (w/v), 0.39% (w/v), 0.4% (w/v), 0.41% (w/v), 0.42% (w/v), 0.43% (w/v), 0.44% (w/v), 0.45% (w/v), 0.46% (w/v), 0.47% (w/v), 0.48% (w/v), 0.49% (w/v), 0.5% (w/v), 0.51% (w/v), 0.52% (w/v), 0.53% (w/v), 0.54% (w/v), 0.55% (w/v), 0.56% (w/v), 0.57% (w/v), 0.58% (w/v), 0.59% (w/v), 0.6% (w/v), 0.61% (w/v), 0.62% (w/v), 0.63% (w/v), 0.64% (w/v), 0.65% (w/v), 0.66% (w/v), 0.67% (w/v), 0.68% (w/v), 0.69% (w/v), 0.7% (w/v), 0.71% (w/v), 0.72% (w/v), 0.73% (w/v), 0.74% (w/v), 0.75% (w/v), 0.76% (w/v), 0.77% (w/v), 0.78% (w/v), 0.79% (w/v), 0.8% (w/v), 0.81% (w/v), 0.82% (w/v), 0.83% (w/v), 0.84% (w/v), 0.85% (w/v), 0.86% (w/v), 0.87% (w/v), 0.88% (w/v), 0.89% (w/v), 0.9% (w/v), 0.91% (w/v), 0.92% (w/v), 0.93% (w/v), 0.94% (w/v), 0.95% (w/v), 0.96% (w/v), 0.97% (w/v), 0.98% (w/v), 0.99% (w/v), or 1% (w/v) benzalkonium chloride.

In certain embodiments, the absorption enhancer is an alkylsaccharide. In certain embodiments, the alkylsaccharide is chosen from dodecyl maltoside, tetradecyl maltoside (TDM) and sucrose dodecanoate.

In certain embodiments, the alkylsaccharide is dodecyl maltoside (the alkylglycoside 1-O-n-dodecyl-β-D-maltopyranoside, alternately referred to as lauryl-β-D-maltopyranoside, dodecyl maltopyranoside, and DDM; C₂₄H₄₆Q₁₁, often referred to by the trade name Intravail®). Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Alkylsaccharides increase absorption by increasing paracellular permeability, as indicated by a decrease in transepithelial electrical resistance; they may also increase transcytosis. The effect is short-lived. Other alkylsaccharides include tetradecyl maltoside (TDM) and sucrose dodecanoate.

In certain embodiments, an intranasal formulation comprises between about 0.05% (w/v) and about 2.5% (w/v) Intravail®. In certain embodiments, an intranasal formulation comprises between about 0.1% (w/v) and about 0.5% (w/v) Intravail®. In certain embodiments, an intranasal formulation comprises between about 0.15% (w/v) and about 0.35% (w/v) Intravail®. In certain embodiments, an intranasal formulation comprises between about 0.15% (w/v) and about 0.2% (w/v) Intravail®. In certain embodiments, an intranasal formulation comprises about 0.18% (w/v) Intravail®. In certain embodiments, an intranasal formulation comprises about 0.2% (w/v) to about 0.3% (w/v) Intravail®. In certain embodiments, an intranasal formulation comprises about 0.25% (w/v) Intravail®.

In certain embodiments, the absorption enhancer is Intravail® (dodecyl maltoside).

In certain embodiments, the absorption enhancer in the intranasal formulation is a combination of dodecyl maltoside and benzalkonium chloride. While the use of dodecyl maltoside or benzalkonium chloride as an absorption enhancer in the intranasal formulations described herein provides bioavailability of intranasal epinephrine, the combination of dodecyl maltoside and benzalkonium chloride as absorption enhancers in the intranasal formulations described herein provides pharmacokinetics that closely match pharmacokinetics obtained through intramuscular injection of epinephrine.

In certain embodiments, each dose dispensed from the device of the pharmaceutical formulation comprises between about 0.4 mg and about 2.40 mg per dose epinephrine, or a salt thereof, and between 0.1 and 0.50 mg Intravail® (dodecyl maltoside).

In certain embodiments, each dose dispensed from the device of the formulation comprises between about 0.5 mg and about 2.0 mg per dose epinephrine, or a salt thereof, and about between 0.1 and 0.50 mg Intravail® (dodecyl maltoside).

In certain embodiments, each dose dispensed from the device of the formulation comprises between about 0.75 mg and about 1.5 mg per dose epinephrine, or a salt thereof, and between 0.1 and 0.50 mg Intravail® (dodecyl maltoside).

In certain embodiments, each dose dispensed from the device of the formulation comprises between about 0.9 mg and about 1.15 mg per dose epinephrine, or a salt thereof, and about 0.25 mg Intravail® (dodecyl maltoside).

In certain embodiments, each dose dispensed from the device of the formulation comprises about 2.5 mg per dose epinephrine, or a salt thereof, and about 0.25 mg Intravail® (dodecyl maltoside). In certain embodiments, each dose dispensed from the device of the formulation comprises about 2.0 mg per dose epinephrine, or a salt thereof, and about 0.25 mg Intravail® (dodecyl maltoside). In certain embodiments, each dose dispensed from the device of the formulation comprises about 1.5 mg per dose epinephrine, or a salt thereof, and about 0.25 mg Intravail® (dodecyl maltoside). In certain embodiments, each dose dispensed from the device of the formulation comprises about 1.0 mg per dose epinephrine, or a salt thereof, and about 0.25 mg Intravail® (dodecyl maltoside). In certain embodiments, each dose dispensed from the device of the formulation comprises about 0.5 mg per dose epinephrine, or a salt thereof, and about 0.25 mg Intravail® (dodecyl maltoside).

In certain embodiments, the pharmaceutical formulation additionally comprises a chelating agent or antioxidant (stabilizing agent) to improve stability. In certain embodiments, the chelating/stabilizing agent is EDTA.

Examples of additional stabilizing agents include: acacia, agar, albumin, alginic acid, aluminum stearate, ammonium alginate, ascorbic acid, ascorbyl palmitate, bentonite, butylated hydroxytoluene (BHT), calcium alginate, calcium stearate, carboxymethylcellulose calcium, carboxymethylcellulose sodium, carrageenan, cellulose, microcrystalline, carboxymethylcellulose sodium, ceratonia, colloidal silicon dioxide, cyclodextrins, diethanolamine, edetates, ethylcellulose, ethylene glycol palmitostearate, glycerin monostearate, guar gum, hectorite, hydroxpropyl betadex, hydroxypropyl cellulose, hypromellose, inulin, invert sugar, lauric acid, lecithin, magnesium aluminum silicate, mineral oil and lanolin alcohols, monoethanolamine, pectin, pentetic acid, phospholipids, polacrilin potassium, poloxamer, polyvinyl alcohol, potassium alginate, potassium chloride, povidone, propyl gallate, propylene glycol, propylene glycol alginate, raffinose, sodium acetate, sodium alginate, sodium borate, sodium stearyl fumarate, sorbitol, stearyl alcohol, sulfobutylether b-cyclodextrin, tagatose, trehalose, triethanolamine, white wax, xanthan gum, xylitol, yellow wax, and zinc acetate.

Examples of additional chelating agents include: citric acid monohydrate, disodium edetate, edetate calcium disodium, edetic acid, fumaric acid, malic acid, maltol, pentetic acid, sodium edetate, and trisodium edetate.

In certain embodiments, the pharmaceutical formulation comprises benzalkonium chloride.

In certain embodiments, the pharmaceutical formulation comprises about 0.005% (w/v) to about 1% (w/v) benzalkonium chloride.

In its capacity as a surfactant, benzalkonium chloride can affect the surface tension of droplets from a delivered nasal spray plume, producing spherical or substantially spherical particles having a narrow droplet size distribution (DSD), as well as the viscosity of a liquid formulation.

In certain embodiments, the absorption enhancer is an alkylsaccharide, for example, a nonionic alkylsaccharide surfactant such as an alkylglycoside and a sucrose ester of fatty acids that consists of an aliphatic hydrocarbon chain coupled to a sugar moiety by a glycosidic or ester bond, respectively. In certain embodiments, the absorption enhancer is an alkylmaltoside (e.g., a tetradecyl maltoside (TDM), a dodecyl maltoside (DDM), etc.). In certain embodiments, the absorption enhancer is sucrose dodecanoate. Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Without being bound to any theory, it is believed that alkylsaccharides increase absorption by increasing paracellular permeability, as indicated by a decrease in transepithelial electrical resistance; they may also increase transcytosis. The effect may be short-lived. In its capacity as an absorption enhancer, alkylmaltosides (e.g., a tetradecyl maltoside (TDM), a dodecyl maltoside (DDM), etc.) can affect the surface tension of droplets from a delivered nasal spray plume, producing spherical or substantially spherical particles having a narrow droplet size distribution (DSD), as well as the viscosity of a liquid formulation.

In certain embodiments, the absorption enhancer is the alkylsaccharide 1-O-n-dodecyl-β-D-maltopyranoside (alternately referred to as lauryl-β-D-maltopyranoside, dodecyl maltopyranoside, dodecyl maltoside, and DDM; C₂₄H₄₆Q₁₁; often referred to by the trade name Intravail®). In certain embodiments, an intranasal formulation comprises about 0.01% (w/v) to about 2.5% (w/v) DDM. In certain embodiments, an intranasal formulation comprises about 0.1% (w/v) to about 0.5% (w/v) DDM. In certain embodiments, an intranasal formulation comprises about 0.15% (w/v) to about 0.35% (w/v) DDM. In certain embodiments, an intranasal formulation comprises about 0.15% (w/v) to about 0.2% (w/v) DDM. In certain embodiments, an intranasal formulation comprises about 0.18% (w/v) DDM. In certain embodiments, an intranasal formulation comprises about 0.2% (w/v) to about 0.3% (w/v) DDM. In certain embodiments, an intranasal formulation comprises about 0.25% (w/v) DDM.

In sugar chemistry, an anomer is either of a pair of cyclic stereoisomers (designated a or (3) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, α-D-glucopyranose and β-D-glucopyranose, the two cyclic forms of glucose, are anomers. Likewise, alkylglycosides occur as anomers. For example, dodecyl β-D-maltoside and dodecyl α-D-maltoside are two cyclic forms of dodecyl maltoside. The two different anomers are two distinct chemical structures, and thus have different physical and chemical properties. In one aspect of the invention, the alkylglycoside of the present invention is a β anomer. In an exemplary aspect, the alkylglycoside is a β anomer of an alkylmaltoside, such as tetradecyl-β-D-maltoside (TDM).

In some embodiments, the alkylglycoside used is a substantially pure alkylglycoside. As used herein a “substantially pure” alkylglycoside refers to one anomeric form of the alkylglycoside (either the α or β anomeric forms) with less than about 2% of the other anomeric form, preferably less than about 1.5% of the other anomeric form, and more preferably less than about 1% of the other anomeric form. In one aspect, a substantially pure alkylgycoside contains greater than 98% of either the α or β anomer. In another aspect, a substantially pure alkylgycoside contains greater than 99% of either the α or β anomer. In another aspect, a substantially pure alkylgycoside contains greater than 99.5% of either the α or β anomer. In another aspect, a substantially pure alkylgycoside contains greater than 99.9% of either the α or β anomer.

In certain embodiments, described herein is an aqueous formulation suitable for intranasal administration comprising: epinephrine; water; and one or more ingredients selected from absorption enhancers, chelating agents, antioxidants, stabilizing agents, surfactants, isotonicity agents, and pH adjusting agents.

In certain embodiments, described herein is an aqueous formulation suitable for intranasal administration comprising: epinephrine; water; and one or more ingredients selected from alkylglycosides, chitosan, alkylcyclodextrins, benzalkonium chloride, sodium chloride, and EDTA.

In certain embodiments, described herein is an aqueous formulation suitable for intranasal administration comprising: epinephrine; water; and one or more ingredients selected from dodecyl maltoside (DDM), tetradecyl maltoside (TDM), benzalkonium chloride, sodium chloride, hydrochloric acid, and EDTA. In certain other embodiments, described herein is an aqueous formulation suitable for intranasal administration comprising: epinephrine; water; and one or more ingredients selected from dodecyl maltoside (DDM), benzalkonium chloride, sodium chloride, and EDTA.

In certain embodiments, described herein is an aqueous formulation suitable for intranasal administration comprising: epinephrine; water; dodecyl maltoside (DDM); and one or more ingredients selected from benzalkonium chloride, sodium chloride, pH adjusting agents, and EDTA.

In certain embodiments, described herein is an aqueous formulation suitable for intranasal administration comprising: epinephrine; water; benzalkonium chloride; and one or more ingredients selected from dodecyl maltoside (DDM), sodium chloride, pH adjusting agents, and EDTA.

In certain embodiments, described herein is an aqueous formulation suitable for intranasal administration comprising: epinephrine; water; dodecyl maltoside (DDM) or benzalkonium chloride or a combination of dodecyl maltoside (DDM) and benzalkonium chloride; and one or more additional ingredients selected from sodium chloride, pH adjusting agents, and EDTA.

pH adjusting agents include acids described herein (e.g. hydrochloric acid, citric acid), buffers (e.g. phosphate, acetate, and citrate buffers), and bases (e.g. sodium hydroxide, sodium citrate, sodium bicarbonate, sodium carbonate).

In certain embodiments, described herein is an aqueous formulation suitable for intranasal administration comprising: about 0.5% (w/v) to about 2.5% (w/v) epinephrine; water; and one or more ingredients selected from: about 0.05% (w/v) to about 2.5% (w/v) dodecyl maltoside (DDM); about 0.005% (w/v) to about 1% (w/v) benzalkonium chloride; about 0.2% (w/v) to about 1.2% (w/v) sodium chloride, optional hydrochloric acid or sodium hydroxide in a sufficient amount to adjust the pH to a final pH of about 4.0 to about 5.0; and about 0.05% (w/v) to about 2.0% (w/v) EDTA.

In certain embodiments, described herein is an aqueous formulation suitable for intranasal administration comprising: i) about 0.5% (w/v) to about 2.5% (w/v) epinephrine; ii) water; iii) about 0.05% (w/v) to about 2.5% (w/v) dodecyl maltoside (DDM) or about 0.005% (w/v) to about 1% (w/v) benzalkonium chloride, or a combination of about 0.05% (w/v) to about 2.5% (w/v) dodecyl maltoside (DDM) and about 0.005% (w/v) to about 1% (w/v) benzalkonium chloride; and iv) one or more ingredients selected from a) about 0.2% (w/v) to about 1.2% (w/v) sodium chloride; b) optional hydrochloric acid or sodium hydroxide in an amount sufficient to adjust the pH to a final pH of about 4.0 to about 5.0; and c) about 0.05% (w/v) to about 2.0% (w/v) EDTA.

In certain embodiments, described herein is an aqueous formulation suitable for intranasal administration comprising: i) about 0.9% (w/v) to about 2.0% (w/v) epinephrine; ii) water; iii) about 0.05% (w/v) to about 2.5% (w/v) dodecyl maltoside (DDM), or about 0.005% (w/v) to about 1% (w/v) benzalkonium chloride, or a combination of about 0.05% (w/v) to about 2.5% (w/v) dodecyl maltoside (DDM) and about 0.005% (w/v) to about 1% (w/v) benzalkonium chloride; and iv) one or more ingredients selected from a) about 0.2% (w/v) to about 1.2% (w/v) sodium chloride; b) optional hydrochloric acid or sodium hydroxide hydrochloric acid in an amount sufficient to adjust the pH to a final pH of about 4.0 to about 5.0; and c) about 0.05% (w/v) to about 2.0% (w/v) EDTA.

In certain embodiments, described herein is an aqueous formulation suitable for intranasal administration comprising: about 0.5% (w/v) to about 2.5% (w/v) epinephrine; water; about 0.05% (w/v) to about 2.5% (w/v) dodecyl maltoside (DDM); about 0.005% (w/v) to about 1% (w/v) benzalkonium chloride; about 0.2% (w/v) to about 1.2% (w/v) sodium chloride, hydrochloric acid in a sufficient amount to adjust the pH to a final pH of about 4.0 to about 5.0; and about 0.05% (w/v) to about 2.0% (w/v) EDTA.

In certain embodiments, described herein is an aqueous formulation suitable for intranasal administration comprising: epinephrine; water; one or more absorption enhancement agents; an isotonicity agent; a stabilizing agent; a preservative; and optional pH adjustment agents to adjust pH to pH 3 to 6. In certain embodiments, described herein is an aqueous formulation suitable for intranasal administration comprising: epinephrine; water; one or more absorption enhancement agents (e.g. dodecyl maltoside; benzalkonium chloride; or a combination of dodecyl maltoside and benzalkonium chloride); an isotonicity agent (e.g. sodium chloride); a stabilizing agent (e.g. EDTA or disodium EDTA); a preservative (e.g. benzalkonium chloride); and optional pH adjustment agents to adjust pH to pH 3 to 6. In certain embodiments, described herein is an aqueous formulation suitable for intranasal administration comprising: epinephrine; water; one or more absorption enhancement agents (e.g. dodecyl maltoside; benzalkonium chloride; or a combination of dodecyl maltoside and benzalkonium chloride); an isotonicity agent (e.g. sodium chloride); a stabilizing agent (e.g. EDTA or disodium EDTA); a preservative (e.g. benzalkonium chloride); an antioxidant; a buffering agent; and optional pH adjustment agents to adjust pH to pH 3 to 6.

In certain embodiments, described herein is an aqueous formulation suitable for intranasal administration comprising: epinephrine; water; dodecyl maltoside or benzalkonium chloride or a combination of dodecyl maltoside and benzalkonium chloride; sodium chloride; EDTA or disodium EDTA; and optional pH adjustment agents to adjust pH to pH 3 to 6.

In certain embodiments, described herein is an aqueous formulation suitable for intranasal administration comprising: about 0.5% (w/v) to about 2.5% (w/v) epinephrine; water; about 0.05% (w/v) to about 2.5% (w/v) dodecyl maltoside or about 0.005% (w/v) to about 1% (w/v) benzalkonium chloride or a combination of about 0.05% (w/v) to about 2.5% (w/v) dodecyl maltoside and about 0.005% (w/v) to about 1% (w/v) benzalkonium chloride; about 0.2% (w/v) to about 1.2% (w/v) sodium chloride; about 0.05% (w/v) to about 2.0% (w/v) EDTA or disodium EDTA; and optional pH adjustment agents to adjust pH to pH 3 to 6.

In some embodiments, a 100 μL sample of the aqueous formulation suitable for intranasal administration comprises less than about 2.5 mg of epinephrine. In some embodiments, a 100 μL sample of the aqueous formulation suitable for intranasal administration comprises about 0.5 mg to about 2.5 mg of epinephrine. In some embodiments, a 100 μL sample of the aqueous formulation suitable for intranasal administration comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg of epinephrine.

In some embodiments, a 100 μL sample of the aqueous formulation suitable for intranasal administration comprises less than about 2.5 mg and greater than about 0.5 mg of epinephrine, and less than about 0.5 mg greater than about 0.01 mg of dodecyl maltoside. In some embodiments, a 100 μL sample of the aqueous formulation suitable for intranasal administration comprises about 0.5 mg to about 2.5 mg of epinephrine, and about 0.01 mg to about 0.5 mg of dodecyl maltoside. In some embodiments, a 100 μL sample of the aqueous formulation suitable for intranasal administration comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg about 1.7 mg, about 1.8 mg, about 1.9 mg, about 2.0 mg, about 2.1 mg, about 2.2 mg, about 2.3 mg about 2.4 mg, or about 2.5 mg of epinephrine; and about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17 mg, about 0.18 mg, about 0.19 mg, about 0.2 mg, about 0.21 mg, about 0.22 mg, about 0.23 mg, about 0.24 mg, about 0.25 mg, about 0.275 mg, about 0.3 mg, about 0.325 mg, about 0.35 mg, about 0.4 mg, about 0.45 mg, or about 0.5 mg of dodecyl maltoside. In some embodiments, a 100 μL sample of the aqueous formulation suitable for intranasal administration comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg of epinephrine; and about 0.2 mg, about 0.21 mg, about 0.22 mg, about 0.23 mg, about 0.24 mg, about 0.25 mg, about 0.275 mg, or about 0.3 mg of dodecyl maltoside. In some embodiments, a 100 μL sample of the aqueous formulation suitable for intranasal administration comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg of epinephrine; and about 0.25 mg.

In some embodiments, a 100 μL sample of the aqueous formulation suitable for intranasal administration comprises less than about 2.5 mg of epinephrine. In some embodiments, a 100 μL sample of the aqueous formulation suitable for intranasal administration comprises about 0.5 mg to about 2.5 mg of epinephrine. In some embodiments, a 100 μL sample of the aqueous formulation suitable for intranasal administration comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg of epinephrine.

### Nasal Drug Delivery Devices and Kits

Also provided are nasal drug delivery devices comprising a formulation described herein. In certain embodiments, the device is pre-primed. In certain embodiments, the device can be primed before use. In certain embodiments, the device can be actuated with one hand.

Nasal delivery is considered an attractive, safe, and easy-to-administer route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions, emulsions, liposomes, and microspheres can also be delivered. Other liquid formulations can comprise liposomes, microspheres, mixed aqueous-organic formulations, non-aqueous formulations, dry powder and retentive formulations (gels). In traditional spray pump systems, antimicrobial preservatives are typically required to maintain microbiological stability in liquid formulations. Metered spray pumps have dominated the nasal drug delivery market since they were introduced. The pumps typically deliver 100 μL (25-250 μL) per spray, and they offer high reproducibility of the emitted dose and plume geometry in in vitro tests.

Examples of standard metered spray pumps include those offered by Aptar Pharma, Inc., such as the multi-dose “classic technology platform” nasal spray devices, and by BD Medical-Pharmaceutical Systems, such as the Accuspray™ system. Such devices comprise a reservoir which holds multiple doses of the nasal spray formulation (e.g., 50, 100, 150, 200, 60, or 120 doses), a closure (e.g., screw, crimp, or snap-on), and an actuator which delivers anywhere from 45 to 1000 μL (e.g. 50, 100, 140, 150, or 200 μL) of fluid per actuation to comprise a single dose. The actuator may be configured to count doses, deliver gel formulations, deliver in an upside-down configuration, etc.

In traditional multi-use spray pump systems, antimicrobial preservatives are typically required to maintain microbiological stability in liquid formulations. However, preservative-free systems are also available, e.g. the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc. Additional nasal spray devices are propellant driven. Yet additional nasal spray devices include dry powder inhalers.

The particle size and plume geometry can vary within certain limits and depend on the properties of the pump, the formulation, the orifice of the actuator, and the force applied. The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 μm. If the droplets are too large (>about 120 μm), deposition takes place mainly in the anterior parts of the nose, and if the droplets are too small (<about 10 μm), they can possibly be inhaled and reach the lungs and oral cavity, which should be avoided because of safety reasons. In its capacity as a surfactant, benzalkonium chloride and alkylmaltosides (e.g., a tetradecyl maltoside (TDM), a dodecyl maltoside (DDM), etc.) can affect the surface tension of droplets from a delivered nasal spray plume, producing spherical or substantially spherical particles having a narrow droplet size distribution (DSD), as well as the viscosity of a liquid formulation.

Plume geometry, droplet size and DSD of the delivered plume subsequent to spraying may be measured under specified experimental and instrumental conditions by appropriate and validated and/or calibrated analytical procedures known in the art. These include photography, laser diffraction, and impaction systems (cascade impaction, NGI). Plume geometry, droplet size and DSD can affect pharmacokinetic outcomes such as Cₘₐₓ, Tₘₐₓ, and dose proportionality.

Droplet size distribution can be controlled in terms of ranges for the D10, D50, D90, span [(D90-D10)/D50], and percentage of droplets less than 10 mm. In certain embodiments, the formulation will have a narrow DSD. In certain embodiments, the formulation will have a D(v,50) of 30-70 μm and a D(v, 90)<100 μm.

In certain embodiments, the percent of droplets less than 10 μm will be less than 10%. In certain embodiments, the percent of droplets less than 10 μm will be less than 5%. In certain embodiments, the percent of droplets less than 10 μm will be less than 2%. In certain embodiments, the percent of droplets less than 10 μm will be less than 1%.

In certain embodiments, the formulation when dispensed by actuation from the device will produce a uniform circular plume with an ovality ratio close to 1. Ovality ratio is calculated as the quotient of the maximum diameter (Dₘₐₓ) and the minimum diameter (Dₘᵢₙ) of a spray pattern taken orthogonal to the direction of spray flow (e.g., from the “top”). In certain embodiments, the ovality ratio is less than ±2.0. In certain embodiments, the ovality ratio is less than ±1.5. In certain embodiments, the ovality ratio is less than ±1.3. In certain embodiments, the ovality ratio is less than ±1.2. In certain embodiments, the ovality ratio is less than ±1.1.

The details and mechanical principles of particle generation for different types of nasal aerosol devices has been described. See, Vidgren and Kublik, Adv. Drug Deliv. Rev. 29:157-77, 1998. Traditional spray pumps replace the emitted liquid with air, and preservatives are therefore required to prevent contamination. However, driven by the studies suggesting possible negative effects of preservatives, pump manufacturers have developed different spray systems that avoid the need for preservatives. These systems use a collapsible bag, a movable piston, or a compressed gas to compensate for the emitted liquid volume (on the World Wide Web at aptar.com and on the World Wide Web at rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip. More recently, pumps have been designed with side-actuation. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (on the World Wide Web at rexam.com and on the World Wide Web at aptar.com).

Traditional, simple single, bi-dose and multi-use metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window of time in which to use the device, particularly if they are not used often. For expensive drugs and drugs intended for single administration or sporadic use and where tight control of the dose and formulation is of importance, single-dose (UDS) or bi-dose spray (BDS) devices are preferred (on the World Wide Web at aptar.com). A simple variant of a single-dose spray device (MAD™) is offered by LMA (LMA, Salt Lake City, UT, USA; on the World Wide Web at lmana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, NC, USA; on the World Wide Web at bdpharma.com) is used to deliver the influenza vaccine FluMist™ (on the World Wide Web at flumist.com), approved for both adults and children in the US market. A similar device for two doses was marketed by a Swiss company for delivery of another influenza vaccine a decade ago.

Pre-primed single- and bi-dose devices are also available, and consist of a reservoir, a piston, and a swirl chamber (see, e.g., the UDS UnitDose™ and BDS BiDose™ devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (on the World Wide Web at gsk.com) and Zomig® (on the World Wide Web at az.com; Pfeiffer/Aptar single-dose device), the marketed influenza vaccine Flu-Mist (on the World Wide Web at flumist.com; Becton Dickinson single-dose spray device), and the intranasal formulation of naloxone for opioid overdose rescue, Narcan Nasal® (on the World Wide Web at narcan.com; Adapt Pharma) are delivered with this type of device.

In certain embodiments, the 90% confidence interval for dose delivered per actuation is about 2%. In certain embodiments, the 95% confidence interval for dose delivered per actuation is ±about 2.5%.

Historically, intranasal administration of drugs in large volume, such as from syringes adapted with mucosal atomizer devices (MADs), has encountered difficulty due to the tendency of some of the formulation to drip back out of the nostril or down the nasopharynx. Accordingly, in certain embodiments, upon nasal delivery of said pharmaceutical formulation to said patient, less than about 20% of said pharmaceutical formulation leaves the nasal cavity via drainage into the nasopharynx or externally. In certain embodiments, upon nasal delivery of said pharmaceutical formulation to said patient, less than about 10% of said pharmaceutical formulation leaves the nasal cavity via drainage into the nasopharynx or externally. In certain embodiments, upon nasal delivery of said pharmaceutical formulation to said patient, less than about 5% of said pharmaceutical formulation leaves the nasal cavity via drainage into the nasopharynx or externally.

Current container closure system designs for inhalation spray drug products include both pre-metered and device-metered presentations using mechanical or power assistance and/or energy from patient inspiration for production of the spray plume. Pre-metered presentations contain previously measured doses or a dose fraction in some type of units (e.g., single or multiple blisters or other cavities) that are subsequently inserted into the device during manufacture or by the patient before use. Typical device-metered units have a reservoir containing formulation sufficient for multiple doses that are delivered as metered sprays by the device itself when activated by the patient.

With aseptic techniques, the use of preservatives may not be required in pre-primed devices, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex™ (sumatriptan) and Zomig™ (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat, irradiation, or chemical (gas). In an aseptic process, the drug product, container, and closure are first subjected to sterilization methods separately, as appropriate, and then brought together. Because there is no process to sterilize the product in its final container, it is critical that containers be filled and sealed in an efficient quality environment. Aseptic processing involves more variables than terminal sterilization. Before aseptic assembly into a final product, the individual parts of the final product generally can be subjected to various sterilization processes. For example, glass containers are subjected to dry heat; rubber closures are subjected to moist heat; and liquid dosage forms are subjected to filtration. Each of these manufacturing processes requires validation and control.

Devices recited herein may employ any of the pharmaceutical formulations, and are useful in the methods disclosed herein.

Accordingly, provided herein are devices adapted for nasal delivery of a pharmaceutical formulation to a patient, comprising a reservoir with a therapeutically effective amount of epinephrine.

In certain embodiments, epinephrine is the only pharmaceutically active compound in the pharmaceutical formulation. In certain embodiments, the pharmaceutical formulation comprises epinephrine and does not include a second pharmaceutically active compound that is a vasodilator or a COMT inhibitor.

In certain embodiments, the volume of the pharmaceutical formulation in the reservoir is not more than about 140 μL.

In certain embodiments, the volume of the pharmaceutical formulation in the reservoir is above about 125 μL and less than 140 μL.

In certain embodiments, about 100 μL of the pharmaceutical formulation in the reservoir is delivered to the patient in one actuation.

In certain embodiments, the volume of the pharmaceutical formulation in the reservoir is above about 125 μL and less than 140 μL and about 100 μL of the pharmaceutical formulation in the reservoir is delivered to the patient in one actuation. In certain embodiments, the volume of the pharmaceutical formulation in the reservoir is above about 120 μL and less than 140 μL and about 100 μL of the pharmaceutical formulation in the reservoir is delivered to the patient in one actuation. In some embodiments, about 100 μL of the pharmaceutical formulation in the reservoir is delivered to the patient in one actuation and comprises less than about 2.5 mg of epinephrine. In some embodiments, about 100 μL of the pharmaceutical formulation in the reservoir is delivered to the patient in one actuation and comprises about 0.5 mg to about 2.5 mg of epinephrine. In some embodiments, about 100 μL of the pharmaceutical formulation in the reservoir is delivered to the patient in one actuation and comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg about 1.6 mg, about 1.7 mg, about 1.8 mg, about 1.9 mg, about 2.0 mg, about 2.1 mg, about 2.2 mg about 2.3 mg, about 2.4 mg, or about 2.5 mg of epinephrine.

In certain embodiments, the pharmaceutical formulation further comprises one or more excipients selected from water, EDTA, and sodium chloride. In certain embodiments, the pharmaceutical formulation further comprises benzalkonium chloride.

In some embodiments, about 100 μL of the aqueous pharmaceutical formulation in the reservoir is delivered to the patient in one actuation and comprises epinephrine, dodeclymaltoside or benzalkonium chloride or a combination of dodeclymaltoside and benzalkonium chloride, EDTA, and NaCl.

In certain embodiments, the pharmaceutical formulation is substantially free of antimicrobial preservatives.

In certain embodiments, the pharmaceutical formulation further comprises a compound which acts as a preservative, absorption enhancer and/or a cationic surfactant; an isotonicity agent; a stabilizing agent; and an amount of acid or base sufficient to achieve a pH of about 3.5 to about 6.0. The use of absorption enhancers, such as alkylsaccharides, cyclodextrins, and chitosans may increase the rate at which epinephrine is absorbed. In general, absorption enhancers provide improved pharmacokinetic outcomes such as increased Cₘₐₓ, reduced Tₘₐₓ, and dose proportionality compared to both intramuscular formulations and intranasal formulations that do not contain an absorption enhancer. Without being bound to any theory, such absorption enhancers typically operate by affecting two primary mechanisms for nasal absorption: paracellular transport via opening of tight junctions between cells, and transcellular transport or transcytosis through cells via vesicle carriers.

Some absorption enhancing excipients can alter the paracellular and/or transcellular pathways, others can extend residence time in the nasal cavity or prevent metabolic changes. Without an absorption enhancer, the molecular-weight limit for nasal absorption is about 1 kDa, while administration of drugs in conjunction with absorption enhancers can enable the absorption of molecules from 1-30 kDa. Intranasal administration of most absorption enhancers, however, can cause nasal mucosa damage. Maggio, J Excipients and Food Chem. 5(2):100-12, 2014. Examples of absorption enhancers include aprotinin, benzalkonium chloride, benzyl alcohol, capric acid, ceramides, cetylpyridinium chloride, chitosan, cyclodextrins, deoxycholic acid, decanoyl camitine, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl camitine, lauryl sulfate, lysophosphatidylcholine, menthol, poloxamer 407, poloxamer F68, poly-L-arginine, polyoxyethylene-9-lauryl ether, polysorbate 80, propylene glycol, quillaia saponin, salicylic acid, β-sitosterol-β-D-glucoside, sucrose cocoate, taurocholic acid, taurodeoxycholic acid, taurodihydrofusidic acid, and alkylsaccharides, such as dodecyl maltoside, tetradecyl maltoside and sucrose dodecanoate.

Epinephrine may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. The acid addition salts may be obtained as the direct products of compound synthesis. In the alternative, the free base may be dissolved in a suitable solvent containing the appropriate acid and the salt isolated by evaporating the solvent or otherwise separating the salt and solvent. The salt may form solvates with standard low molecular weight solvents using methods known to the skilled artisan.

In certain embodiments, the device is filled with the pharmaceutical formulation using sterile filling.

In certain embodiments, the pharmaceutical formulation is chemically storage-stable for about twelve months at about 25° C. and about 60% relative humidity and about six months at about 40° C. and about 75% relative humidity.

In some embodiments, intranasal epinephrine is delivered as an aqueous solution, aqueous suspension, aqueous emulsion, non-aqueous solution, non-aqueous suspensions, non-aqueous emulsion, a solution with halogenated hydrocarbon propellant(s), or as a dry powder. In some embodiments, aqueous formulations are sprayed into the nostril. In some embodiments, aqueous formulations are aerosolized by liquid nebulizers employing either hydraulic or ultrasonic atomization. Propellant-based systems may use suitable pressurized metered-dose inhalers (pMDIs). Dry powders may use dry powder inhaler devices (DPIs), which are capable of dispersing the drug substance effectively.

Propellants typically used include chlorofluorocarbons, hydrochlorofluorocarbons, hydrofluorocarbons, hydrocarbons, and compressed gases.

In some embodiments, intranasal epinephrine is delivered as a nasal aerosol produced by a nasal pressurized metered-dose inhalers (pMDIs). In some embodiments, the pMDI is a hydrofluoroalkane (HFA)-based pMDI for nasal use. Like spray pumps, nasal pMDIs produce a localized deposition on the anterior non-ciliated epithelium of the nasal vestibule and in the anterior parts of the narrow nasal valve, but due to quick evaporation of the spray delivered with a pMDI, noticeable “drip-out” may be less of an issue.

In some embodiments, epinephrine is delivered with a nebulizer. Nebulizers use compressed gasses (air, oxygen, and nitrogen) or ultrasonic or mechanical power to break up medical solutions and suspensions into small aerosol droplets that can be directly inhaled into the nose. The smaller particles and slow speed of the nebulized aerosol increase penetration to the target sites in the middle and superior meatuses and the paranasal sinuses.

In some embodiments, epinephrine is delivered with a pulsating aerosol generated via a perforated vibrating membrane. In some embodiments, the pulsation membrane nebulizer is VibrENT (PARI Pharma GmbH). In some embodiments, epinephrine is delivered with a pulsating aerosol in combination with breathing techniques

In some embodiments, epinephrine is delivered with Bi-Directional™ delivery technology (e.g. Bi-Directional™ Exhalation Delivery Systems (EDS); OptiNose).

In some embodiments, epinephrine is delivered with an atomizer. In some embodiments, the atomizer is a handheld battery-driven atomizer intended for nasal drug delivery. In some embodiments, the atomizer atomizes liquids by producing a vortical flow on the droplets as they exit the device. Such devices include the ViaNase™ atomizer (by Kurve Technology Inc., Lynnwood, WA, USA). In some embodiments, the atomizer is a nasal atomizer driven by highly pressurized nitrogen gas.

In some embodiments, intranasal epinephrine is delivered with a nasal powder device. In some embodiments, the nasal powder device is a nasal powder inhaler, nasal powder sprayer, or nasal powder insufflator. Powder sprayers typically have a compressible compartment to provide a pressure that when released creates a plume of powder particles fairly similar to that of a liquid spray. Breath-actuated inhalers require the user to use his or her own breath to inhale the powder into the nostril from a blister or capsule. Nasal insufflator devices consist of a mouthpiece and a nosepiece that are fluidly connected. Delivery occurs when the subject exhales into the mouthpiece to close the velum, and the airflow carries the powder particles into the nose through the device nosepiece.

In some embodiments, the nasal powder inhaler is a blister based powder inhaler. Typically, the blister is pierced before use and the device nosepiece placed into one nostril. The subject closes the other nostril with the finger and inhales the powder into the nose. Representative devises include BiDose™/Prohaler™, and Twin-lizer™.

Representative nasal powder sprayers include, but are not limited to, UnidoseDP™, Fit-lizer™, Monopowder™, SoluVent™)

In some embodiments, the nasal powder sprayer is a capsule-based, single-dose powder devices. In one such embodiment, the capsule-based, single-dose powder device consist of a chamber that cuts off the top and bottom of the capsule when inserted. A plastic chamber is compressed by hand, compressed air passes through a one-way valve and the capsule during actuation, and the powder is emitted.

In some embodiments, the nasal powder sprayer consists of an air-filled compartment that is compressed until a pin ruptures a membrane to release pressure that emits a plume of powder.

In some embodiments, the nasal powder sprayer consists of a plunger that when pressed creates a positive pressure that ruptures a membrane to expel the powder.

In some embodiments, the nasal powder insufflator requires the subject to blow into one end of the tube while the other end is inserted into the vestibule of the nostril.

In some embodiments, intranasal epinephrine is delivered with a breath-powered Bi-Directional™ delivery device. A breathpowered Bi-Directional™ nasal delivery device utilizes the exhaled breath to deliver the drug into the nose. Breath-powered Bi-Directional™ devices consist of a mouthpiece and a sealing nosepiece with an optimized frusto-conical shape and comfortable surface that mechanically expands the first part of the nasal valve. The user slides a sealing nosepiece into one nostril until it forms a seal with the flexible soft tissue of the nostril opening, at which point, it mechanically expands the narrow slit-shaped part of the nasal triangular valve. The user then exhales through an attached mouthpiece. When exhaling into the mouthpiece against the resistance of the device, the soft palate (or velum) is automatically elevated by the positive oropharyngeal pressure, isolating the nasal cavity from the rest of the respiratory system. Owing to the sealing nosepiece, the dynamic pressure that is transferred from the mouth through the device to the nose further expands the slit-like nasal passages. This “breath-powered” mechanism enables release of liquid or powder particles into an air stream that enters one nostril, passes entirely around the nasal septum, and exits through the opposite nostril. Actuation of drug release in devices employing this approach use manual triggering or mechanisms automatically triggered by flow and/or pressure.

### Single-Dose Devices

In certain embodiments, the device is a single-dose device, wherein the pharmaceutical formulation is present in one reservoir, and wherein the therapeutically effective amount of the epinephrine is delivered essentially by one actuation of the device.

Also provided herein is a single-use, pre-primed device adapted for nasal delivery of a pharmaceutical formulation to a patient by one actuation of the device into one nostril of the patient, having a single reservoir comprising about 100 μL of a pharmaceutical formulation as disclosed herein.

In certain embodiments, the device is actuatable with one hand.

In certain embodiments, the delivery time is less than about 30 seconds. In certain embodiments, the delivery time is less than about 25 seconds. In certain embodiments, the delivery time is less than about 20 seconds. In certain embodiments, the delivery time is less than about 15 seconds.

In certain embodiments, the 90% confidence interval for dose delivered per actuation is about 2%. In certain embodiments, the 95% confidence interval for dose delivered per actuation is ±about 2.5%.

In certain embodiments, upon nasal delivery of the formulation to the patient, less than about 20%, less than about 15%, less than about 10%, or less than about 5%, of the formulation leaves the nasal cavity via drainage into the nasopharynx or externally, as provided above.

In certain embodiments, said formulation is chemically storage-stable for about twelve months at about 25° C. and about 60% relative humidity and/or about six months at about 40° C. and about 75% relative humidity.

### Bi-Dose Devices

In certain embodiments, said device is a bi-dose device, wherein a first volume of said formulation is present in a first reservoir and a second volume of said formulation is present in a second reservoir, and wherein said therapeutically effective amount is delivered essentially by a first actuation of said device into a first nostril of said patient and a second actuation of said device into a second nostril of said patient.

In certain embodiments, said first volume and said second volume combined is equal to not more than about 380 μL.

In certain embodiments, about 100 μL of said first volume of said formulation is delivered by said first actuation.

In certain embodiments, about 100 μL of said second volume of said formulation is delivered by said second actuation.

In certain embodiments, said bi-dose device is actuatable with one hand.

In certain embodiments, the delivery time is less than about 30 seconds. In certain embodiments, the delivery time is less than about 25 seconds. In certain embodiments, the delivery time is less than about 20 seconds. In certain embodiments, the delivery time is less than about 15 seconds.

In certain embodiments, the 90% confidence interval for dose delivered per actuation is about 2%. In certain embodiments, the 95% confidence interval for dose delivered per actuation is ±about 2.5%.

In certain embodiments, upon nasal delivery of the formulation to the patient, less than about 20%, less than about 15%, less than about 10%, or less than about 5%, of the formulation leaves the nasal cavity via drainage into the nasopharynx or externally.

Also provided are embodiments wherein any embodiment above may be combined with any one or more of these embodiments, provided the combination is not mutually exclusive.

### Indications

Also provided are formulations and devices for use in treating conditions mediated by adrenergic receptors, and/or one or more symptoms thereof, and methods of treatment of such conditions comprising administering the formulations and using the devices disclosed herein.

In certain embodiments, the condition is (1) treatment of acute hypersensitivity, such as a type-1 hypersensitivity reaction (for example such as an anaphylactoid reaction (systemic allergic reaction) to foods, drugs, animal serums, insect bites and stings, and other allergens, see below), (2) treatment of acute asthmatic attacks to relieve bronchospasm not controlled by inhalation or subcutaneous administration of other solutions of the drug, (3) treatment and prophylaxis of cardiac arrest and/or attacks of transitory atrioventricular (A-V) heart block with syncopal seizures (Stokes-Adams Syndrome), (4) to increase mean arterial blood pressure in adult patients with hypotension associated with septic shock, (5) for induction and maintenance of mydriasis during intraocular surgery.

In certain embodiments, the type-1 hypersensitivity reaction (systemic allergic reaction) is chosen from allergic asthma, allergic conjunctivitis, allergic rhinitis (hay fever), anaphylaxis, angioedema, urticaria (hives), eosinophilia, antibiotic allergy (e.g. to penicillin or cephalosporin), and food allergy (e.g. to peanuts or shellfish).

In certain embodiments, the type-1 hypersensitivity reaction (systemic allergic reaction) is anaphylaxis.

Symptoms of anaphylaxis include hives, generalized itching, nasal congestion, wheezing, difficulty breathing, cough, cyanosis, lightheadedness, dizziness, confusion, slurred speech, rapid pulse, palpitations, nausea and vomiting, abdominal pain or cramping, skin redness or inflammation, nasal flaring, and intercostal retractions.

In certain embodiments, the symptom of the type-1 hypersensitivity reaction (systemic allergic reaction) is chosen from generalized hives (urticatria), itching (pruritis), flushing, swelling (angioedema) of the afflicted tissues, a burning sensation of the skin (common in those with angioedema), swelling of the tongue or throat, respiratory symptoms such as shortness of breath, wheezes, or stridor shortness of breath, coronary artery spasm, myocardial infarction, dysrhythmia, or cardiac arrest (those with underlying coronary disease are at greater risk of cardiac effects), tachycardia, bradycardia, and a Bezold-Jarisch reflex.

In certain embodiments, the type-1 hypersensitivity reaction (systemic allergic reaction) is caused by stinging insects (e.g., order Hymenoptera, which include bees, wasps, hornets, yellow jackets and fire ants), biting insects (e.g., triatoma, mosquitoes), allergen immunotherapy, foods, drugs, diagnostic testing substances (e.g. radiocontrast media) and other allergens, as well as idiopathic anaphylaxis or exercise-induced anaphylaxis.

In certain embodiments, the cardiac arrest is out-of-hospital cardiac arrest.

In certain embodiments, the type-1 hypersensitivity reaction (systemic allergic reaction) is urticaria. In certain embodiments, the type-1 hypersensitivity reaction (systemic allergic reaction) is a periodic acute exacerbation of urticaria. In certain embodiments, the type-1 hypersensitivity reaction (systemic allergic reaction) is a periodic acute exacerbation of urticaria in a patient whose symptoms of chronic spontaneous urticaria are typically controlled with antihistamines and/or anti-IgE antibodies.

Chronic spontaneous urticaria (CSU), also known as chronic hives with no known external trigger, is a condition characterized by hives, swelling, or both for more than 6 weeks. Chronic idiopathic urticaria is defined by the presence of wheals, angioedema, or both for more than six weeks. The most common symptoms of chronic spontaneous urticaria are angioedema and hives that are accompanied by itchiness. With severe or long-lasting CSU, other symptoms like headache; fatigue; joint pain or swelling; sudden reddening of your face, neck, or upper chest; wheezing; stomach symptoms like diarrhea; or a rapid heartbeat, may be experienced. Chronic spontaneous urticaria is typically treated with antihistamines (e.g., cetirizine, fexofenadine, loratadine, cyproheptadine, diphenhydramine, doxepin, or hydroxyzine), steroids followed by antihistamines; or antacid pills, anti-inflammatory antibiotics, or biologics (e.g., anti-IgE antibodies), followed by optional steroids and/or optional antihistamines.

While it is natural to look for causes for your chronic hives, CSU isn't caused by an allergy or external trigger, so what you ate, wore, or touched does not start or trigger your flares, but some may worsen symptoms. Chronic hives is thought to be linked to an overactive immune system. It is different from other forms of hives or swelling that can be triggered by external factors such as foods, cold, heat, and sweat.

Chronic hives can show up anywhere on the body. They are sometimes accompanied by swelling, also called angioedema, of the lips, eyelids, hands, feet, and other areas. Both hives and swelling can make people feel miserable and impact many aspects of their lives.

Also provided are embodiments wherein any embodiment above may be combined with any one or more of these embodiments, provided the combination is not mutually exclusive.

### EXAMPLES

The following examples are provided for illustrative purposes only and not to limit the scope of the claims provided herein.

### Example 1. Epinephrine Formulations for Clinical Use

A representative procedure for the preparation of formulations for clinical use is described. The Formulation Excipient Solution (FES) can be made in advance (up to 7 days) and stored at room temperature. The epinephrine stock solution (ESS) should be made fresh within 72 hours of dosing, protected from light and excessive oxidation and stored at 2-8° C. until 2 hours before use. A mixture of equal volumes of sterile filtered FES and ESS will result in a solution of epinephrine, dodecylmaltoside (DDM), EDTA, benzalkonium chloride (BZK) in saline for use in the clinical protocols below.

A 200 mL batch of Formulation Excipient Solution (FES) is prepared by weighing 0.80 g (0.75-0.85 g) of EDTA into a 200 mL volumetric flask and dissolving in −150 mL of Sterile Saline; weighing 1.00 g (0.95-1.05 g) of Intravail® DDM, quantitatively transferring to the EDTA solution, and mixing until dissolved (solution should be clear and colorless); if necessary, using gentle heating (40-60° C.) aid solution, then cooling to room temperature once dissolved; adding the desired amount of a BZK solution (or adding BZK as a solid) and adding to the mixing Intravail®/EDTA mixture; adding the appropriate amount of 1 N HCl to attain a pH of 4 (e.g. approximately 20 mL), and diluting QS to volume with Sterile Saline, and stirring until the mixture is uniform. The pH of the FES solution may be measured and recorded.

Epinephrine Stock Solution (ESS) 10 mg/mL should be freshly prepared, protected from light (e.g. with foil, the use of brown colored lights, etc.), and use within 72 hours of dosing. To formulate a 100 mL batch of final 10 mg/mL product: ensure 100 mL volumetric flask is wrapped in foil prior to adding FES Solution; add 50 mL of FES Solution to each of two foil wrapped 100 mL flasks (50 mL per flask); weigh and add 1.0 g (0.95-1.05 g) of epinephrine (E4250 Sigma Aldrich) into each of the two 100 mL flasks; mix each until uniform; measure the pH of each flask and record.

Final Dosing Formulations (FDF) are prepared by filling appropriate sprayers capable of delivering 100 μL per spray with appropriate amounts of ESS (e.g. about 5.0 mL of ESS for Aptar multi-dose spray devices or about 125 μL of ESS for uni-dose spray devices).

Representative epinephrine formulations for clinical use are presented in Table 2, Table 3, and Table 4.

TABLE 2
Representative Epinephrine Formulations for Clinical Use.

| Ingredients | Quantity per mL | | | | | |
|---|---|---|---|---|---|---|
| (−)-Epinephrine | 3 | 5 | 10 | 10 | 10 | 20 |
| USP (mg) | | | | | | |
| DDM (mg) | 2.5 | 2.5 | 2.5 | 2.5 | 2.5 | 2.5 |
| Disodium EDTA | 2.0 | 2.0 | 2.0 | 2.0 | 2.0 | 2.0 |
| USP (mg) | | | | | | |
| BZK USP (mg) | 0.1 | 0.2 | 0.2 | 0.4 | 0.6 | 0.2 |
| Sodium chloride | 8.23 | 8.23 | 8.23 | 8.23 | 8.23 | 8.23 |
| USP (mg) | | | | | | |
| 1N HCl (mL) | 0.051 | 0.051 | 0.051 | 0.051 | 0.051 | 0.051 |
| 0.1N HCl and/or | Adjust to | Adjust to | Adjust to | Adjust to | Adjust to | Adjust to |
| 0.1 NaOH | pH 3.8-4.2 | pH 3.8-4.2 | pH 3.8-4.2 | pH 3.8-4.2 | pH 3.8-4.2 | pH 3.8-4.2 |
| Purified water, | QS to 1 mL | QS to 1 mL | QS to 1 mL | QS to 1 mL | QS to 1 mL | QS to 1 mL |
| Millipore, Type I | | | | | | |

TABLE 3
Representative Epinephrine Formulations for Clinical Use.

| Ingredients | Quantity per mL | | | | | | |
|---|---|---|---|---|---|---|---|
| (−)-Epinephrine | 3 | 5 | 6.5 | 10 | 13 | 15 | 20 |
| USP (mg) | | | | | | | |
| DDM (mg) | 2.5 | 2.5 | 2.5 | 2.5 | 2.5 | 2.5 | 2.5 |
| Disodium EDTA | 2.0 | 2.0 | 2.0 | 2.0 | 2.0 | 2.0 | 2.0 |
| USP (mg) | | | | | | | |
| BZK USP (mg) | 0.4 | 0.4 | 0.4 | 0.4 | 0.4 | 0.4 | 0.4 |
| Sodium chloride | 8.23 | 8.23 | 8.23 | 8.23 | 8.23 | 8.23 | 8.23 |
| USP (mg) | | | | | | | |
| 1N HCl (mL) | 0.051 | 0.051 | 0.051 | 0.051 | 0.051 | 0.051 | 0.051 |
| 0.1N HCl and/or | Adjust to | Adjust to | Adjust to | Adjust to | Adjust to | Adjust to | Adjust to |
| 0.1 NaOH | pH 3.8-4.2 | pH 3.8-4.2 | pH 3.8-4.2 | pH 3.8-4.2 | pH 3.8-4.2 | pH 3.8-4.2 | pH 3.8-4.2 |
| Purified water, | QS to 1 mL | QS to 1 mL | QS to 1 mL | QS to 1 mL | QS to 1 mL | QS to 1 mL | QS to 1 mL |
| Millipore, Type I | | | | | | | |

TABLE 4
Representative Epinephrine Formulations for Clinical Use.

| Ingredients | Quantity per mL | | | | | | | |
|---|---|---|---|---|---|---|---|---|
| (−)-Epinephrine USP | 10 | 10 | 10 | 10 | 10 | 10 | 10 | 10 |
| (mg) | | | | | | | | |
| Dodecylmaltoside | 2.5 | 2.5 | 2.5 | 2.5 | 2.5 | 2.5 | 2.5 | 2.5 |
| (DDM) (mg) | | | | | | | | |
| Disodium EDTA USP | 2.0 | 2.0 | 2.0 | 2.0 | 2.0 | 2.0 | 2.0 | 2.0 |
| (mg) | | | | | | | | |
| Benzalkonium Chloride | 0.4 | 0.4 | 0.4 | 0.4 | 0.4 | 0.4 | 0.4 | 0.4 |
| USP (mg) | | | | | | | | |
| Sodium chloride USP | 8.23 | 8.23 | 8.23 | 8.23 | 8.23 | 8.23 | 8.23 | 8.23 |
| (mg) | | | | | | | | |
| Butylated | — | 0.1 | 0.1 | — | — | — | — | — |
| hydroxyanisole (BHA) | | | | | | | | |
| (mg) | | | | | | | | |
| Citric acid | — | — | 0.42 | — | 4.2 | — | 4.2 | — |
| monohydrate (mg) | | | | | | | | |
| Isoascorbic Acid (mg) | — | — | — | 0.1 | 0.1 | — | — | |
| D-α-Tocopherol | — | — | — | — | — | 5.0 | 5.0 | — |
| polyethylene glycol | | | | | | | | |
| 1000 succinate (mg) | | | | | | | | |
| Sodium metabisulfite | — | — | — | — | — | — | — | 0.05 |
| (mg) | | | | | | | | |
| 1N HCl | 0.051 mL | 0.051 mL | 0.051 mL | 0.051 mL | 0.051 mL | 0.051 mL | 0.051 mL | 0.051 mL |
| 0.1N HCl | Adjust | Adjust | Adjust | Adjust | Adjust | Adjust | Adjust | Adjust |
| | to pH | to pH | to pH | to pH | to pH | to pH | to pH | to pH |
| | 3.8-4.2 | 3.8-4.2 | 3.8-4.2 | 3.8-4.2 | 3.8-4.2 | 3.8-4.2 | 3.8-4.2 | 3.8-4.2 |
| 0.1 NaOH | Adjust | Adjust | Adjust | Adjust | Adjust | Adjust | Adjust | Adjust |
| | to pH | to pH | to pH | to pH | to pH | to pH | to pH | to pH |
| | 3.8-4.2 | 3.8-4.2 | 3.8-4.2 | 3.8-4.2 | 3.8-4.2 | 3.8-4.2 | 3.8-4.2 | 3.8-4.2 |
| Purified water, | QS to 1 | QS to 1 | QS to 1 | QS to 1 | QS to 1 | QS to 1 | QS to 1 | QS to 1 |
| Millipore, Type I | mL | mL | mL | mL | mL | mL | mL | mL |

TABLE 5
Representative Epinephrine Formulations for Clinical Use.

| Ingredients | Quantity per mL | | | | | | | |
|---|---|---|---|---|---|---|---|---|
| (−)-Epinephrine USP | 20 | 20 | 20 | 20 | 20 | 20 | 20 | 20 |
| (mg) | | | | | | | | |
| Dodecylmaltoside | 2.5 | 2.5 | 2.5 | 2.5 | 2.5 | 2.5 | 2.5 | 2.5 |
| (DDM) (mg) | | | | | | | | |
| Disodium EDTA USP | 2.0 | 2.0 | 2.0 | 2.0 | 2.0 | 2.0 | 2.0 | 2.0 |
| (mg) | | | | | | | | |
| Benzalkonium Chloride | 0.4 | 0.4 | 0.4 | 0.4 | 0.4 | 0.4 | 0.4 | 0.4 |
| USP (mg) | | | | | | | | |
| Sodium chloride USP | 8.23 | 8.23 | 8.23 | 8.23 | 8.23 | 8.23 | 8.23 | 8.23 |
| (mg) | | | | | | | | |
| Butylated | — | 0.1 | 0.1 | — | — | — | — | — |
| hydroxyanisole (BHA) | | | | | | | | |
| (mg) | | | | | | | | |
| Citric acid | — | — | 0.42 | — | 4.2 | — | 4.2 | — |
| monohydrate (mg) | | | | | | | | |
| Isoascorbic Acid (mg) | — | — | — | 0.1 | 0.1 | — | — | |
| D-α-Tocopherol | — | — | — | — | — | 5.0 | 5.0 | — |
| polyethylene glycol | | | | | | | | |
| 1000 succinate (mg) | | | | | | | | |
| Sodium metabisulfite | — | — | — | — | — | — | — | 0.05 |
| (mg) | | | | | | | | |
| 1N HCl | 0.051 mL | 0.051 mL | 0.051 mL | 0.051 mL | 0.051 mL | 0.051 mL | 0.051 mL | 0.051 mL |
| 0.1N HCl | Adjust | Adjust | Adjust | Adjust | Adjust | Adjust | Adjust | Adjust |
| | to pH | to pH | to pH | to pH | to pH | to pH | to pH | to pH |
| | 3.8-4.2 | 3.8-4.2 | 3.8-4.2 | 3.8-4.2 | 3.8-4.2 | 3.8-4.2 | 3.8-4.2 | 3.8-4.2 |
| 0.1 NaOH | Adjust | Adjust | Adjust | Adjust | Adjust | Adjust | Adjust | Adjust |
| | to pH | to pH | to pH | to pH | to pH | to pH | to pH | to pH |
| | 3.8-4.2 | 3.8-4.2 | 3.8-4.2 | 3.8-4.2 | 3.8-4.2 | 3.8-4.2 | 3.8-4.2 | 3.8-4.2 |
| Purified water, | QS to 1 | QS to 1 | QS to 1 | QS to 1 | QS to 1 | QS to 1 | QS to 1 | QS to 1 |
| Millipore, Type I | mL | mL | mL | mL | mL | mL | mL | mL |

### Example 2: Previous Clinical Studies

Prior studies with neffy in healthy volunteers, in patients with Type I allergies, in patients with allergic rhinitis, in patients with upper respiratory tract infection (URTI), and pediatric patients with Type 1 allergies.

### Pharmacokinetics of Neffy 1.0 mg

neffy is a solution formulation of epinephrine intended for nasal administration. neffy contains 10 mg/mL of epinephrine, 2.5 mg/mL of dodecylmaltoside (DDM), 2.0 mg/mL of disodium EDTA, 0.4 mg/mL of benzalkonium chloride, in 0.9% saline, water for injection QS to 1.0 mL, HCl/NaOH to adjust pH 4.0-4.5). neffy is administered by IN administration using the commercial sprayer device at a dose of 1.0 mg epinephrine per spray delivered in a volume of 100 μL aqueous solution. neffy will be provided in the Unit Dose Sprayer (UDS).

Blood samples for the measurement of epinephrine plasma concentrations were collected at the following timepoints for each treatment period: predose (at −10 and −5 minutes) and at 2, 4, 6, 8, 10, 12.5, 15, 20, 30, 45, 60, 90, 120, 150, 180, 240, 360, and 480 minutes after dosing. For all studies, only the first dose of neffy 1.0 mg dose was used.

The PK parameters were calculated without the subtraction of the pre-dose epinephrine concentrations as the primary PK analysis. The rationale for using epinephrine plasma levels without subtraction of the baseline values is based on both clinical and PK considerations. First, the endogenous levels of epinephrine change based on both the diurnal cycle and due to stimuli. Second, clinically, the absolute plasma levels are considered more important to elicit an efficacious response.

Mean epinephrine versus time curves and a summary of key PK parameters are provided in Table 6.

TABLE 6
Comparison of PK Parameters

| | | | Median tₘₐₓ | | AUC₀₋ₜ |
|---|---|---|---|---|---|
| | | Cₘₐₓ (pg/mL) | (minutes) | | (min*pg/mL) |
| Product | N | Mean (CV %) | (range) | | Mean (CV %) |
| neffy 1.0 mg | 135 | 258 (69.8) | 30.0 | (0.00-150) | 23700 (59.1) |
| Epinephrine 0.3 mg IM | 104 | 254 (58.4) | 45.0 | (0.00-360) | 27200 (38.4) |
| Symjepi 0.3 mg | 36 | 438 (64.6) | 30.0 | (4.00-90.0) | 23700 (37.5) |
| EpiPen 0.3 mg | 71 | 503 (73.5) | 20.0 | (3.00-154) | 27900 (43.9) |

The highest mean concentration occurred after administration via EpiPen, followed by Symjepi, manual epinephrine 0.3 mg IM with needle and syringe (Epinephrine 0.3 mg IM), and neffy. The highest maximum plasma concentration (Cₘₐₓ) values were observed following EpiPen followed by Symjepi. The Cₘₐₓ values were comparable between following neffy 1.0 mg and Epinephrine 0.3 mg IM. The longest median time to maximum plasma concentration (tₘₐₓ) occurred following Epinephrine 0.3 mg IM (45.0 minutes). The shortest median tₘₐₓ occurred following EpiPen (20.0 minutes). The area under the plasma concentration-time curve (AUC₀₋ₜ) value appeared comparable among all treatments.

Although the PK profile of neffy was comparable to that of Epinephrine 0.3 mg IM.

### Pharmacokinetics of Neffy 2.0 Mg

neffy is a solution formulation of epinephrine intended for nasal administration. neffy contains 20 mg/mL of epinephrine, 2.5 mg/mL of dodecylmaltoside (DDM), 2.0 mg/mL of disodium EDTA, 0.4 mg/mL of benzalkonium chloride, in 0.9% saline, water for injection QS to 1.0 mL, HCl/NaOH to adjust pH 4.0-4.5). neffy is administered by IN administration using the commercial sprayer device at a dose of 2.0 mg epinephrine per spray delivered in a volume of 100 μL aqueous solution. neffy will be provided in the Unit Dose Sprayer (UDS).

Blood samples for the measurement of epinephrine plasma concentrations were collected at the following timepoints for each treatment period: predose (at −10 and −5 minutes) and at 2, 4, 6, 8, 10, 12.5, 15, 20, 30, 45, 60, 90, 120, 150, 180, 240, 360, and 480 minutes after dosing. For all studies, only the first dose of neffy was used.

The PK summary statistics including pediatric, self-administration and rhinitis condition for once and/or twice-dosed treatments are presented in Table 7 and Table 8, respectively.

TABLE 7
Summary Statistics of Total Epinephrine PK Parameters: Once-Dosed Treatments

| | | | | tₘₐₓ (min) | | | |
|---|---|---|---|---|---|---|---|
| | | Cₘₐₓ (pg/mL) | | median | | AUCₗₐₛₜ (min*pg/mL) | |
| Treatment | N | Mean(% CV) | Geo. mean | (range) | | Mean(% CV) | Geo. mean |
| neffy 2.0 mg IN | 78 | 485 (70.6) | 361 | 20.5 | (2--150) | 40900 (67.5) | 32600 |
| Epinephrine 0.3 mg | 178 | 277 (65.4) | 234 | 45 | (3.9-360) | 27900 (38.7) | 26100 |
| IM | | | | | | | |
| EpiPen 0.3 mg | 77 | 581 (75.6) | 447 | 10 | (2--45) | 31600 (39.3) | 29200 |
| neffy 2.0 mg IN | 42 | 421 (66.4) | 332 | 30 | (6--240) | 46776 (55.9) | 38884 |
| (self-administration) | | | | | | | |
| neffy 2.0 mg IN | 16 | 540 (70.7) | 433 | 25.0 | (2.5--120) | 35500 (76.3) | 27800 |
| (pediatrics) | | | | | | | |
| neffy 2.0 mg IN | 33 | 309 (66.2) | 260 | 6 | (2--90) | 23500 (69.1) | 19700 |
| (with rhinitis) | | | | | | | |

TABLE 8
Summary Statistics of Total Epinephrine PK Parameters: Twice-Dosed Treatments

| | | | | | tₘₐₓ (min) | | | |
|---|---|---|---|---|---|---|---|---|
| | | Cₘₐₓ (pg/mL) | | | median | AUCₗₐₛₜ (min*pg/mL) | | |
| Treatment | N | Mean(% CV) | | Geo. mean | (range) | Mean (% CV) | | Geo. mean |
| neffy 2.0 mg | 39 | 1000 | (93.1) | 706 | 30 (6--150) | 86000 | (77) | 66700 |
| twice (L/R) | | | | | | | | |
| neffy 2.0 mg | 39 | 992 | (75.3) | 729 | 30 (4--150) | 86500 | (60.5) | 69900 |
| twice (R/R) | | | | | | | | |
| Epinephrine 0.3 | 70 | 436 | (48.8) | 386 | 45 (6--180) | 47500 | (32.6) | 45300 |
| mg IM twice | | | | | | | | |
| EpiPen 0.3 mg | 78 | 754 | (64.7) | 630 | 20 (4--360) | 55000 | (47.9) | 29200 |
| twice | | | | | | | | |

The PK profile of neffy 2.0 mg single dose was within the range of that of injection products. In twice dosing (Table 8), neffy 2 mg was dose proportional and similar between once in each nostril (L/R) or twice in one nostril (R/R). The Cₘₐₓ from neffy 2 mg dosed twice were similar to 0.3 mg EpiPen dosed twice. In all studies, IM injection regardless of device used did not result in proportional increases in exposures.

### Example 3: A Five-Treatment, Five-Period, Randomized Crossover Study of the Pharmacokinetics and Pharmacodynamics of Epinephrine after Repeat Administration of Neffy and Intramuscular Epinephrine Injection in Type I Allergy Patients with Seasonal Rhinitis

### Objectives

### Primary Objectives

To evaluate the comparative pharmacokinetics (PK) of two doses of neffy 2.0 mg with IM 0.3 mg under allergic rhinitis conditions compared to two dose administration under normal conditions.

To evaluate the comparative pharmacodynamics (PD) of two doses of neffy 2.0 mg with IM 0.3 mg based on systolic blood pressure (SBP), diastolic blood pressure (DBP) and pulse rate (PR) using an automated blood pressure measuring (ABPM) device under allergic rhinitis conditions compared to two dose administration under normal conditions.

### Secondary Objectives

To evaluate the comparative PK/PD relationship of neffy 2 mg after two doses under normal and allergic rhinitis conditions.

To evaluate the comparative PK/PD relationship of IM 0.3 mg after two doses under normal and allergic rhinitis conditions.

To evaluate the safety and tolerability of neffy in subjects as compared to IM 0.3 mg.

### Study Design

This is a Phase 1, five-treatment period, randomized, cross-over study that will consist of a screening period, baseline period, and an open-label treatment period. Subjects enrolled will have diagnosed and confirmed seasonal rhinitis (Type 1 allergy patients) with known etiology.

Comparative PKs of two doses of neffy and IM 0.3 mg will be assessed in subjects both in a normal state and under allergic rhinitis.

Approximately 46 eligible subjects, 18 to 64 years of age, with seasonal allergies (positive case history of seasonal allergic rhinitis related to tree or grass allergens with positive skin prick test within 12 months of enrollment and positive nasal allergen challenge [NAC] with Allergen Panel at Screening between 21 and 60 days prior to treatment in the study) will be enrolled in the five treatment periods. Subjects will have a Total Nasal Symptom Score (TNSS) of 5 out of 12 and a congestion score of ≥2 out of 3 on at least one test allergen during the screening NAC.

A diagram of the study design is below.

Screening Period: Subjects will have an assessment for response to their previously diagnosed target antigen (standardized HollisterStier allergen solutions) based on TNSS between 21 and 60 days in advance of study treatment. An initial nasal wash with saline and phenol will be used to exclude subjects with nonspecific hyperresponsiveness. Subjects reporting a TNSS response greater than 2 from the diluent dose will be excluded. Allergen concentration will be prepared from stock solutions (HollisterStier) in serial dilutions, using a diluent solution of saline and phenol, from 1:128 to 1:2; each concentration will be a 4-fold dilution of the previous. Subjects are challenged in both nostrils with escalating (fourfold) increments of allergen at 15 min (±5 min) intervals until a positive reaction (i.e., TNSS of 5 out of 12 and a congestion score of 2 out of 3) is reached. If the subject reaches the highest concentration of 1:2 without qualifying, the subject will be excluded. Once a positive reaction is confirmed, any further dosing will be stopped, and rescue medicine may be given. The qualifying concentration will be used for the Treatment Periods 3, 4, and 5. Subjects who meet criteria (qualified) by TNSS score at screening at a dilution of 1:128 will receive a dilution of 1:25.6 upon dosing in Treatment Periods 3, 4, and 5. Subjects who meet criteria (qualified) by TNSS score at screening at a dilution of 1:32 will receive a dilution of 1:6.1 upon dosing in Treatment Periods 3, 4, and 5.

All subjects will undergo safety screening evaluations within 60 days prior to treatment in the study.

Baseline/Open-Label Treatment Period: Approximately forty-six (46) eligible subjects will be enrolled in the Treatment Periods 1-5.

In Treatment Periods 1-2, the PKs of two doses of neffy 2.0 mg (4.0 mg total dose) will be assessed as compared to two doses of IM 0.3 mg under normal nasal conditions. Each subject will be randomized to receive each of the following treatments and then cross-over to the other product treatment: Two doses of neffy 2.0 mg (4.0 mg total dose) in the right and left naris administered 10 minutes apart, Two doses of IM 0.3 mg in the right and left anterolateral thigh administered 10 minutes apart.

In Treatment Periods 3-5, the PKs of two doses of neffy 2.0 mg (4.0 mg total dose) will be assessed as compared to two doses of IM 0.3 mg after induction of rhinitis via NAC. Each subject will receive each of the following treatments in Periods 3-5: Two doses of IM 0.3 mg in the left and right anterolateral thigh administered 10 minutes apart, Two doses of neffy 2.0 mg (4.0 mg total dose) in the left and right naris administered 10 minutes apart, Two doses of neffy 2.0 mg (4.0 mg total dose) in the right and right naris administered 10 minutes apart.

NAC will be conducted with the cumulative dilution concentration that was established based on the Screening visit. The cumulative dilution concentration will be calculated per the table below.

Treatments will be separated by a 24-hour wash out period in the Treatment Periods 1-3 and by 3 weeks between the Treatment Periods 3 and 4 as well as 4 and 5.

Blood samples (4 mL each) for the measurement of plasma concentrations of epinephrine will be collected at pre-dose and post dose.

During Treatment Periods 1-5, TNSS will be evaluated but subjects will not be discontinued based on TNSS.

PD measurements will include SBP, DBP, and PR after each dosing arm. ABPM equipment capable of measurements each 4-5 minutes with printout capability will be employed.

Safety assessments will be performed on each study day for 6 hours after each dosing. Safety assessments include adverse event (AE) and vital signs. Concomitant medications will be recorded.

Serum samples at pre-NAC (at −45 or −30 minutes before dosing) and at 4 and 30 minutes post dose will be collected for serum tryptase levels for exploratory purposes in Treatment Periods 3, 4, and 5.

### Study Endpoints

### Primary Endpoints

The primary endpoints of this study are as follows: Comparative PK of a 4 mg dose of neffy in subjects with and without induced allergic rhinitis and to evaluate the impact of rhinitis condition on the absorption of epinephrine. Comparative PD of twice dosing of neffy and IM 0.3 mg based on SBP, DBP and PR in subjects with and without induced allergic rhinitis. Comparative PK of twice dosing of neffy 2.0 mg (4 mg total) with twice dosing of IM 0.3 mg (0.6 mg total) under allergic rhinitis conditions. Comparative PD of twice dosing of neffy 2.0 mg (4 mg total) with twice dosing of IM 0.3 mg (0.6 mg total) based on SBP, DBP and PR using an ABPM device under allergic rhinitis conditions.

### Secondary Endpoints

The secondary endpoints of this study are as follows: Comparative PK/PD relationship of neffy 2 mg after twice (4 mg) dosing under normal and allergic rhinitis conditions. Comparative PK/PD relationship of IM 0.3 mg after twice (0.6 mg) dosing under normal and allergic rhinitis conditions. Safety and tolerability of neffy in subjects as compared to IM 0.3 mg.

### Study Drugs

### Test Product

neffy is a solution formulation of epinephrine intended for nasal administration. neffy contains 20 mg/mL of epinephrine, 2.5 mg/mL of dodecylmaltoside (DDM), 2.0 mg/mL of disodium EDTA, 0.4 mg/mL of benzalkonium chloride, in 0.9% saline, water for injection QS to 1.0 mL, HCl/NaOH to adjust pH 4.0-4.5). neffy is administered by IN administration using the commercial sprayer device at a dose of 2.0 mg epinephrine per spray delivered in a volume of 100 μL aqueous solution. neffy will be provided in the Unit Dose Sprayer (UDS).

Intramuscular (IM) Epinephrine 0.3 mg injection with needle and syringe (IM 0.3 mg) was sourced from a commercial supplier for this study.

Dosing of neffy 2.0 mg and IM 0.3 mg will be administered according to the Instructions for Use (IFU) provided for the product or label. If there is excessive nasal drip after NAC, the nose can be wiped before dosing of study drugs.

### Sample Size

46 subjects (patients) diagnosed with seasonal allergic rhinitis and an identifiable antigen

### Study Population

Type I allergy patients with seasonal allergic rhinitis to tree or grass allergens age 18 to 64 years (positive case history with positive skin prick within 12 months of enrollment and NAC between 21 and 60 days prior to study treatment).

### Main Inclusion Criteria

Subjects are eligible to be included in the study only if all of the following criteria are met: is a subject between the ages of 18 and 64 years, inclusive.

Has positive screen for seasonal rhinitis: History of seasonal allergic rhinitis. Positive skin prick (wheal≥3 mm over negative control) and/or intradermal test to allergen(s) within 12 months of enrollment. Subjects will have a TNSS of ≥5 out of 12 and a congestion score of ≥2 out of 3 on at least one card with NAC at screening.

Has body weight more than 50 kg and body mass index between 18 and 35 kg/m², inclusive. Has no medical history of hypertension and cardiovascular disease in the last 10 years. At screening has stable vital signs in the following ranges (after 5 minutes of rest): SBP≥90 and ≤140 mmHg, DBP≥50 and ≤90 mmHg, Heart rate (HR)≥45 and ≤100 beats per minute (bpm).

Is a nonsmoker within the previous 2 months and does not use nicotine-containing products.

Is willing and able to provide written informed consent prior to participating in the study.

### Main Exclusion Criteria

Subjects must not meet any of the following Exclusion criteria to be eligible for enrollment: Has nasal conditions that could interfere with nasal spray administration. Has mucosal inflammatory disorders (e.g., pemphigus or Sjogren's syndrome or fungal sinusitis). Known hypersensitivity to any compound in the test product, or any other closely related compound (e.g., dihydropyridine-derived molecules). Has participated in a clinical trial within 30 days prior to the first dose of study drug. Has had treatment with any epinephrine or norepinephrine containing products within 7 days of Day 1. Has any clinically significant medical condition or physical exam (PE) finding as deemed inappropriate by the Investigator.

### Restrictions

Subjects are excluded from the study if any of the following criteria apply: Take treatment with any known strong or moderate inhibitors or inducers of metabolizing enzymes (e.g., CYP-P450 enzymes or MAO) within fourteen (14) days of Day −1 and during the study. Use nasal decongestants within three (3) days prior to Day −1 and the duration of the study. Has used any systemic steroid containing products (excluding topical steroids) in the past 30 days prior to Day −1 and the duration of the study.

### PK/PD Study Results

Data from the primary analysis of the completed study showed that responses on pharmacodynamic (PD) surrogate markers for efficacy in anaphylaxis, such as systolic blood pressure and heart rate, correlated well with pharmacokinetic (PK) exposures and were consistently higher for repeat doses of neffy, irrespective of dosing in the same nostril (R/R) or opposite nostrils (L/R), compared to repeat doses of IM injection. Dosing in the same nostril (R/R) resulted in higher PD than injection at all time points measured, while dosing in the opposite nostril (L/R) was higher than injection until the 60 minute time point, after which PD was indistinguishable from injection.

Consistent with prior studies, significant responses on these PD surrogate markers of efficacy with neffy were observed even at one minute after dosing. The PD responses demonstrate that the epinephrine exposures achieved with repeat doses of neffy (R/R or L/R) fully activate the receptors involved in reversing anaphylaxis symptoms.

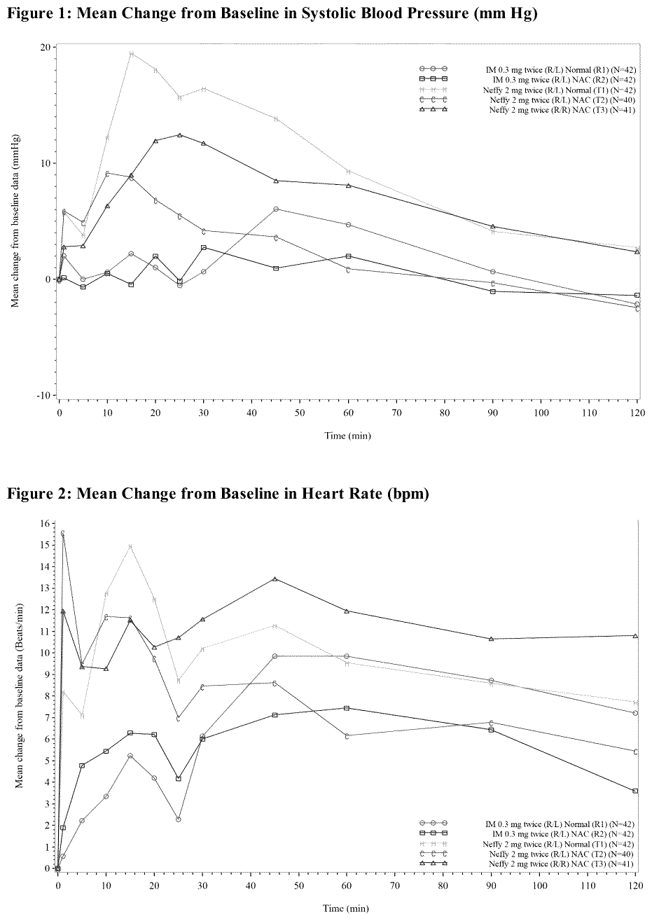

FIG. 1 shows the mean change from baseline in systolic blood pressure (mm Hg).

FIG. 2 shows the mean change from baseline in heart rate (bpm).

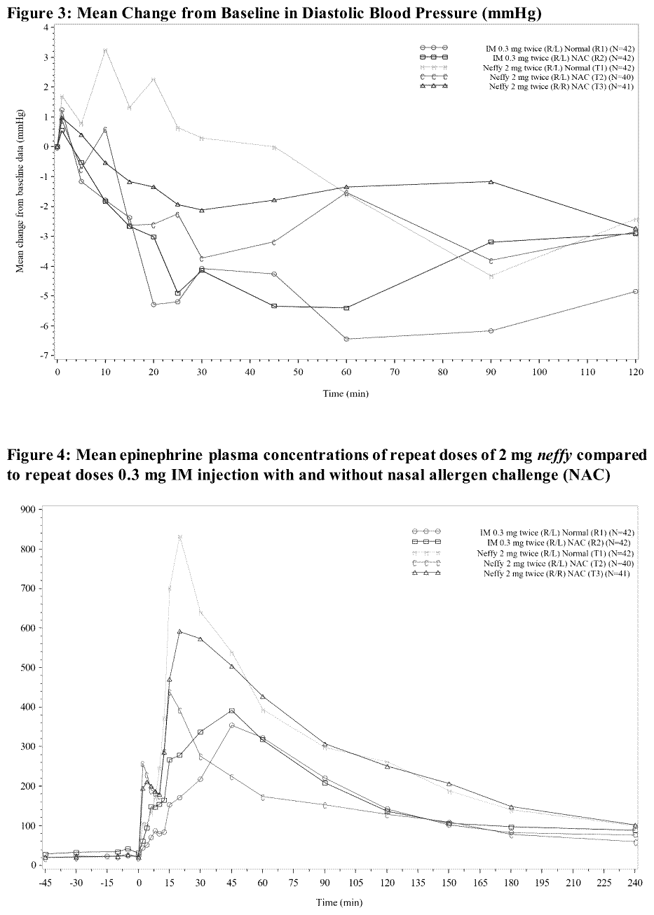

FIG. 3 shows the mean change from baseline in diastolic blood pressure (mmHg).

The clinical data also demonstrated that mean epinephrine concentrations following repeat doses of 2 mg neffy in the same nostril (R/R) were numerically higher than repeat doses of 0.3 mg injection at all time points through at least 240 minutes under NAC conditions.

Mean epinephrine concentrations of 2 mg neffy in the opposite nostril (L/R) were also numerically higher than repeat doses of 0.3 mg injection for approximately 30 minutes and during the time period when clinical response would be expected to be observed after dosing epinephrine (i.e., within 10 minutes).

The PK profile of repeat doses of neffy during normal nasal conditions was shown in prior studies to be highly similar to repeat doses of EpiPen, which serves as the upper bracket for exposures established to be safe. In this study, the PK profile of repeat doses of neffy in the same nostril (R/R) during nasal allergen challenge was highly similar to repeat doses of neffy during normal nasal conditions, and therefore also in the range of exposures established to be safe.

FIG. 4 shows the mean epinephrine plasma concentrations of repeat doses of 2 mg neffy compared to repeat doses 0.3 mg IM injection with and without nasal allergen challenge (NAC).

Other clinical studies have evaluated neffy PK/PD in people with the same TNSS and congestion severity scores as the experimental NAC studies, but to real-world conditions such as upper respiratory tract infections or acute allergic rhinitis from natural causes, showed no meaningful difference on PK/PD compared to dosing neffy under normal nasal conditions.

Repeat doses of neffy were considered safe and well-tolerated. All adverse events were mild and continue to be consistent with those observed in prior studies of neffy across more than 700 subjects. There were no serious adverse events.

Although the present invention has been described with reference to specific details of certain embodiments thereof in the above examples, it will be understood that modifications and variations are encompassed within the spirit and scope of the invention.

What is claimed is:

1. A method of treating a type-1 hypersensitivity reaction in a human comprising intranasally administering to the human with the type-1 hypersensitivity reaction two or more doses of a pharmaceutical formulation of epinephrine, or a salt thereof;
wherein each dose comprises between about 1.0 mg and about 2.0 mg of epinephrine, or a pharmaceutically acceptable salt thereof, and dodecyl maltoside;
wherein the intranasal administration of two doses provides epinephrine pharmacokinetics in the human that is at least the same as the intramuscular injection of two 0.3 mg epinephrine doses in the anterolateral thigh;
wherein the intranasal administration of each dose after the first dose provides dose proportional epinephrine pharmacokinetics;
wherein each dose is intranasally administered in the same nostril; or
wherein each dose is intranasally administered in opposite or alternating nostrils.

2. The method of claim 1, wherein:
each intranasally administered dose of the pharmaceutical formulation provides plasma epinephrine concentrations in the human that are efficacious for the elimination of, and/or for the prevention of, the further progression of, one, more than one, or all of the symptoms of the type-1 hypersensitivity reaction in the human; or
the intranasal administration of the first dose of the pharmaceutical formulation to the human with the type-1 hypersensitivity reaction provides inadequate plasma epinephrine concentrations in the human that are efficacious for the elimination of, and/or for the prevention of, the further progression of, one, more than one, or all of the symptoms of the type-1 hypersensitivity reaction in the human; and
the intranasal administration of the second dose of the pharmaceutical formulation to the human with the type-1 hypersensitivity reaction provides plasma epinephrine concentrations in the human that are efficacious for the elimination of, and/or for the prevention of the further progression of, one, more than one, or all of the symptoms of the type-1 hypersensitivity reaction in the human.

3. The method of claim 1 or claim 2, wherein the human with the type-1 hypersensitivity reaction has symptoms of rhinitis; wherein the symptoms of rhinitis comprise nasal edema, congestion, or both.

4. The method of any one of claims 1-3, wherein the type-1 hypersensitivity reaction comprises allergic asthma, allergic conjunctivitis, allergic rhinitis, anaphylaxis, angioedema, urticaria, eosinophilia, or combinations thereof.

5. The method of any one of claims 1-3, wherein the type-1 hypersensitivity reaction comprises allergic asthma, allergic conjunctivitis, allergic rhinitis, anaphylaxis, angioedema, urticaria, eosinophilia, drug allergy or a food allergy.

6. The method of any one of claims 1-3, wherein the type-1 hypersensitivity reaction comprises an antibiotic allergy

7. The method of any one of claims 1-3, wherein the type-1 hypersensitivity reaction comprises anaphylaxis.

8. The method of any one of claims 1-3, wherein the type-1 hypersensitivity reaction comprises an allergic reaction to an insect sting or bite, venom, allergen immunotherapy, foods, drugs, diagnostic testing substances or other allergens, or combinations thereof.

9. The method of any one of claims 1-3, wherein the type-1 hypersensitivity reaction comprises idiopathic anaphylaxis or exercise-induced anaphylaxis.

10. The method of any one of claims 1-3, wherein the type-1 hypersensitivity reaction comprises anaphylaxis; and the symptoms of anaphylaxis are selected from the group consisting of hives, generalized itching, nasal congestion, wheezing, difficulty breathing, cough, cyanosis, lightheadedness, dizziness, confusion, slurred speech, rapid pulse, palpitations, nausea or vomiting, abdominal pain or cramping, skin redness or skin inflammation, nasal flaring, and intercostal retractions.

11. The method of any one of claims 1-3, wherein the type-1 hypersensitivity reaction comprises urticaria, and the symptoms of the type-1 hypersensitivity reaction comprise pruritis, flushing, or a burning sensation of the skin.

12. The method of any one of claims 1-11, wherein intranasal administration of two doses provides pharmacokinetics that are greater than intramuscular injection of two 0.3 mg doses in the anterolateral thigh.

13. The method of any one of claims 1-12, wherein the intranasal administration of the two doses provides plasma epinephrine concentrations with a Cₘₐₓ of at least 100 pg/mL, at least 200 pg/mL, or at least 300 pg/mL.

14. The method of any one of claims 1-13, wherein the intranasal administration of the two doses provides plasma epinephrine concentrations with a time to maximum epinephrine plasma concentrations (Tmax) of less than 45 minutes, less than 35 minutes, less than 25 minutes, or less than 15 minutes.

15. The method of any one of claims 1-14, wherein the pharmaceutical formulation is a nasal spray pharmaceutical formulation, and wherein the pharmaceutical formulation is a nasal spray pharmaceutical formulation comprising between about 1 mg/mL and about 40 mg/mL of epinephrine, or a salt thereof, in a volume between about 25 μL and about 250 μL per dose.

16. The method of any one of claims 1-15, wherein each dose of the pharmaceutical formulation comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL, of epinephrine, or a salt thereof, in a volume between about 25 μL and about 250 μL per dose;
one or more other agents as excipients selected from the group consisting of isotonicity agents, stabilizing agents, antioxidants, preservatives, and pH adjustment agents to adjust the pH to a final pH between about 3.0 and about 5.5; and water.

17. The method of any one of claims 1-15, wherein each dose of the pharmaceutical formulation comprises about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL of epinephrine, or a salt thereof, in a volume between about 50 μL and about 200 μL per dose;
one or more other agents as excipients selected from the group consisting of isotonicity agents, stabilizing agents, antioxidants, preservatives, and pH adjustment agents to adjust the pH to a final pH between about 3.0 and about 5.5; and water.

18. The method of any one of claims 1-15, wherein each dose of the pharmaceutical formulation comprises about 5 mg/mL, about 6 mg/mL, about 7 mg/mL, about 8 mg/mL, about 9 mg/mL, or about 10 mg/mL of epinephrine, or a salt thereof, in a volume of about 200 μL per dose;
one or more other agents as excipients selected from the group consisting of isotonicity agents, stabilizing agents, antioxidants, preservatives, and pH adjustment agents to adjust the pH to a final pH between about 3.0 and about 5.5; and water.

19. The method of any one of claims 1-15, wherein each dose of the pharmaceutical formulation comprises about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof, in a volume of about 100 μL per dose;
one or more other agents as excipients selected from the group consisting of isotonicity agents, stabilizing agents, antioxidants, preservatives, and pH adjustment agents to adjust the pH to a final pH between about 3.0 and about 5.5; and water.

20. The method of any one of claims 1-15, wherein each dose of the pharmaceutical formulation comprises about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL of epinephrine, or a salt thereof, in a volume of about 50 μL per dose;
one or more other agents as excipients selected from the group consisting of isotonicity agents, stabilizing agents, antioxidants, preservatives, and pH adjustment agents to adjust the pH to a final pH between about 3.0 and about 5.5; and water.

21. The method of any one of claims 1-15, wherein each dose of the pharmaceutical formulation comprises about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg of epinephrine, or a salt thereof, in a volume between about 50 μL and about 250 μL per dose;
one or more other agents as excipients selected from the group consisting of isotonicity agents, stabilizing agents, antioxidants, preservatives, and pH adjustment agents to adjust the pH to a final pH between about 3.0 and about 5.5; and water.

22. The method of any one of claims 16-21, wherein the isotonicity agents are selected from the group consisting of dextrose, glycerin, mannitol, potassium chloride, and sodium chloride;
wherein the stabilizing agent is ethylenediaminetetraacetic acid (EDTA) or disodium salt thereof;
wherein the antioxidants are selected from the group consisting of alpha tocopherol, D-α-tocopherol polyethylene glycol 1000 succinate, ascorbic acid, isoascorbic acid, butylated hydroxyanisole, citric acid monohydrate, potassium metabisulfite, sodium bisulfite, sodium metabisulfite, and sodium sulfite;
wherein the preservative is benzalkonium chloride; and
wherein the pH adjustment agents are selected from the group consisting of adipic acid, ammonium chloride, citric acid, acetic acid, hydrochloric acid, lactic acid, phosphoric acid, propionic acid, sulfuric acid, tartaric acid, sodium hydroxide, sodium citrate, sodium bicarbonate, and sodium carbonate.

23. The method of any one of claims 16-21, wherein the isotonicity agents are selected from the group consisting of dextrose, glycerin, mannitol, potassium chloride, and sodium chloride;
wherein the stabilizing agent is ethylenediaminetetraacetic acid (EDTA) or disodium salt thereof;
wherein the antioxidants are selected from the group consisting of alpha tocopherol, ascorbic acid, isoascorbic acid, potassium metabisulfite, sodium bisulfite, and sodium metabisulfite, sodium sulfite;
wherein the preservative is benzalkonium chloride; and
wherein the pH adjustment agents are selected from the group consisting of hydrochloric acid and sodium hydroxide.

24. The method of any one of claims 1-14, wherein each intranasally delivered dose of the pharmaceutical formulation comprises:
about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof;
dodecyl maltoside;
sodium chloride;
optionally sodium metabisulfite;
optionally ethylenediaminetetraacetic acid (EDTA) or disodium salt thereof;
optionally benzalkonium chloride;
hydrochloric acid, sodium hydroxide, or both, in amounts to adjust the pH to a final pH between about 3.0 and about 5.5; and
water;
in a volume between about 100 μL and about 140 μL.

25. The method of any one of claims 1-14, wherein each intranasally delivered dose of the pharmaceutical formulation comprises:
about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof;
about 2.0 mg/mL to about 3.0 mg/mL of dodecyl maltoside;
about 5 to about 10 mg/mL of sodium chloride as an excipient;
hydrochloric acid, sodium hydroxide, or both, in amounts to adjust the pH to a final pH between about 3.0 and about 5.5;
optionally about 0.1 mg/mL to about 4.0 mg/mL of ethylenediaminetetraacetic acid (EDTA) or disodium salt thereof;
optionally about 0.1 mg/mL to about 0.8 mg/mL of benzalkonium chloride as an excipient;
optionally about 0.01 mg/mL to about 0.1 mg/mL of sodium metabisulfite as an excipient; and
water;
in a volume between about 100 μL and about 140 μL.

26. The method of any one of claims 1-14, wherein each intranasally delivered dose of the pharmaceutical formulation comprises:
about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof;
dodecyl maltoside;
sodium chloride;
optionally sodium metabisulfite;
optionally ethylenediaminetetraacetic acid (EDTA) or disodium salt thereof;
optionally benzalkonium chloride;
hydrochloric acid, sodium hydroxide, or both, in amounts to adjust the pH to a final pH between about 3.0 and about 5.5; and
water;
in a volume between about 100 μL and about 140 μL.

27. The method of any one of claims 1-14, wherein each intranasally delivered dose of the pharmaceutical formulation comprises:
about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof;
about 2.0 mg/mL to about 3.0 mg/mL of dodecyl maltoside;
about 5 to about 10 mg/mL of sodium chloride as an excipient;
hydrochloric acid, sodium hydroxide, or both, in amounts to adjust the pH to a final pH between about 3.0 and about 5.5;
optionally about 0.1 mg/mL to about 4.0 mg/mL of ethylenediaminetetraacetic acid (EDTA) or disodium salt thereof;
optionally about 0.1 mg/mL to about 0.8 mg/mL of benzalkonium chloride as an excipient;
optionally about 0.01 mg/mL to about 0.1 mg/mL of sodium metabisulfite as an excipient; and
water;
in a volume between about 100 μL and about 140 μL.

28. The method of any one of claims 24-27, wherein:
each dose of the nasal spray is intranasally administered with a nasal spray device; and one actuation of the nasal spray device delivers about 100 μL of the pharmaceutical formulation.

29. The method of any one of claims 1-14, wherein each intranasally administered dose comprises:
about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof;
dodecyl maltoside;
sodium chloride;
optionally sodium metabisulfite;
optionally ethylenediaminetetraacetic acid (EDTA) or disodium salt thereof;
optionally benzalkonium chloride;
hydrochloric acid, sodium hydroxide, or both, in amounts to adjust the pH to a final pH between about 3.0 and about 5.5; and
water;
in a volume between about 100 μL.

30. The method of any one of claims 1-14, wherein each intranasally administered dose comprises:
about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of epinephrine, or a salt thereof;
about 2.0 mg/mL to about 3.0 mg/mL of dodecyl maltoside;
about 5 to about 10 mg/mL of sodium chloride as an excipient;
hydrochloric acid, sodium hydroxide, or both, in amounts to adjust the pH to a final pH between about 3.0 and about 5.5;
optionally about 0.1 mg/mL to about 4.0 mg/mL of ethylenediaminetetraacetic acid (EDTA) or disodium salt thereof;
optionally about 0.1 mg/mL to about 0.8 mg/mL of benzalkonium chloride as an excipient;
optionally about 0.01 mg/mL to about 0.1 mg/mL of sodium metabisulfite as an excipient; and
water;
in a volume between about 100 μL.

31. A method of treating a type-1 hypersensitivity reaction in a human comprising intranasally administering to the human with the type-1 hypersensitivity reaction two or more doses of a pharmaceutical formulation of epinephrine, or a salt thereof; wherein epinephrine, or a pharmaceutically acceptable salt thereof, is the only pharmaceutically active ingredient in the nasal spray pharmaceutical formulation;
wherein each dose comprises between about 0.1 mg and about 5.0 mg of epinephrine, or a pharmaceutically acceptable salt thereof.

32. The method of claim 31, wherein the two or more doses of the pharmaceutical formulation are intranasally administered in the same nostril.

33. The method of claim 31, wherein the each dose of the pharmaceutical formulation is intranasally administered in opposite or alternating nostrils.

34. A method of treating a type-1 hypersensitivity reaction in a human with an inadequate response to an intranasally administered dose of epinephrine, or a salt thereof, comprising intranasally administering to the human with the type-1 hypersensitivity reaction a second dose of a pharmaceutical formulation of epinephrine, or a salt thereof;
wherein each dose comprises between about 0.1 mg and about 5.0 mg of epinephrine, or a pharmaceutically acceptable salt thereof;
wherein epinephrine, or a pharmaceutically acceptable salt thereof, is the only pharmaceutically active ingredient in each dose of the nasal spray pharmaceutical formulation.

35. The method of claim 34, wherein the second dose is intranasally administered in the same nostril as the first nostril that received the first dose.

36. The method of claim 34, wherein the second dose is intranasally administered in the opposite nostril as the first nostril that received the first dose.

37. A method of enhancing the exposure to a second intranasally administered dose of epinephrine in a human in need of treatment for a type-1 hypersensitivity reaction comprising intranasally administering to the human with the type-1 hypersensitivity reaction two doses of a pharmaceutical formulation of epinephrine, or a salt thereof;
wherein each dose comprises between about 0.1 mg and about 5.0 mg of epinephrine, or a pharmaceutically acceptable salt thereof;
wherein epinephrine, or a pharmaceutically acceptable salt thereof, is the only pharmaceutically active ingredient in the nasal spray pharmaceutical formulation;
wherein the second dose is intranasally administered in the same nostril as the first nostril.

38. The method of any one of claims 31-37, wherein: the intranasal administration of each dose of the pharmaceutical formulation to the human with the type-1 hypersensitivity reaction provides plasma epinephrine concentrations in the human that are efficacious for the elimination of one, more than one, or all of the symptoms of the type-1 hypersensitivity reaction in the human.

39. The method of any one of claims 31-37, wherein: the intranasal administration of the first dose of the pharmaceutical formulation to the human with the type-1 hypersensitivity reaction provides inadequate plasma epinephrine concentrations in the human that are efficacious for the elimination of one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the elimination of one, more than one, or all of the symptoms of the type-1 hypersensitivity reaction in the human.

40. The method of any one of claims 31-39, wherein: the intranasal administration of each dose of the pharmaceutical formulation to the human with the type-1 hypersensitivity reaction provides plasma epinephrine concentrations in the human that are efficacious for the prevention of the further progression of one, more than one, or all of the symptoms of the type-1 hypersensitivity reaction in the human.

41. The method of any one of claims 31-39, wherein: the intranasal administration of the first dose of the pharmaceutical formulation to the human with the type-1 hypersensitivity reaction provides inadequate plasma epinephrine concentrations in the human that are efficacious for the prevention of the further progression of one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the prevention of the further progression of one, more than one, or all of the symptoms of the type-1 hypersensitivity reaction in the human.

42. The method of any one of claims 31-37, wherein: the intranasal administration of the first dose of the pharmaceutical formulation to the human with the type-1 hypersensitivity reaction provides plasma epinephrine concentrations in the human that are efficacious for the elimination of one, more than one, or all of the symptoms of the type-1 hypersensitivity reaction in the human; and the intranasal administration of the second dose of the pharmaceutical formulation to the human with the type-1 hypersensitivity reaction provides plasma epinephrine concentrations in the human that are efficacious for the prevention of the further progression of one, more than one, or all of the symptoms of the type-1 hypersensitivity reaction in the human.

43. The method of any one of claims 31-42, wherein the human with the type-1 hypersensitivity reaction has symptoms of rhinitis; wherein the symptoms of rhinitis comprise nasal edema, congestion, or both.

44. The method of any one of claims 31-43, wherein the type-1 hypersensitivity reaction comprises allergic asthma, allergic conjunctivitis, allergic rhinitis, anaphylaxis, angioedema, urticaria, eosinophilia, or combinations thereof.

45. The method of any one of claims 31-43, wherein the type-1 hypersensitivity reaction comprises allergic asthma, allergic conjunctivitis, allergic rhinitis, anaphylaxis, angioedema, urticaria, eosinophilia, drug allergy or a food allergy.

46. The method of any one of claims 31-43, wherein the type-1 hypersensitivity reaction comprises an antibiotic allergy

47. The method of any one of claims 31-43, wherein the type-1 hypersensitivity reaction comprises anaphylaxis.

48. The method of any one of claims 21-43, wherein the type-1 hypersensitivity reaction comprises an allergic reaction to: an insect sting or bite, venom, allergen immunotherapy, foods, drugs, diagnostic testing substances or other allergens, or combinations thereof.

49. The method of any one of claims 31-43, wherein the type-1 hypersensitivity reaction comprises idiopathic anaphylaxis or exercise-induced anaphylaxis.

50. The method of any one of claims 31-43, wherein the type-1 hypersensitivity reaction comprises anaphylaxis; and the symptoms of anaphylaxis are selected from the group consisting of hives, generalized itching, nasal congestion, wheezing, difficulty breathing, cough, cyanosis, lightheadedness, dizziness, confusion, slurred speech, rapid pulse, palpitations, nausea or vomiting, abdominal pain or cramping, skin redness or skin inflammation, nasal flaring, and intercostal retractions.

51. The method of any one of claims 31-43, wherein the type-1 hypersensitivity reaction comprises urticaria, and the symptoms of the type-1 hypersensitivity reaction comprise pruritis, flushing, or a burning sensation of the skin.

52. The method of any one of claims 31-51, wherein intranasal administration of the two doses provides pharmacokinetics that are at least the same as intramuscular injection of two 0.3 mg doses in the anterolateral thigh.

53. The method of any one of claims 31-51, wherein intranasal administration of the two doses provides pharmacokinetics that are greater than intramuscular injection of two 0.3 mg doses in the anterolateral thigh.

54. The method of any one of claims 31-53, wherein the intranasal administration of the two doses provides plasma epinephrine concentrations with a Cₘₐₓ of at least 100 pg/mL.

55. The method of any one of claims 31-53, wherein the intranasal administration of the two doses provides plasma epinephrine concentrations with a Cₘₐₓ of at least 200 pg/mL.

56. The method of any one of claims 31-53, wherein the intranasal administration of the two doses provides plasma epinephrine concentrations with a Cₘₐₓ of at least 300 pg/mL.

57. The method of any one of claims 31-53, wherein the intranasal administration of the two doses provides plasma epinephrine concentrations with a time to maximum epinephrine plasma concentrations (Tmax) of less than 45 minutes.

58. The method of any one of claims 31-57, wherein the intranasal administration of the two doses provides plasma epinephrine concentrations in the human that are efficacious for the treatment of the type-1 hypersensitivity reaction of with a time to maximum epinephrine plasma concentrations (Tmax) of less than 35 minutes.

59. The method of any one of claims 31-57, wherein the intranasal administration of the two doses provides plasma epinephrine concentrations in the human that are efficacious for the treatment of the type-1 hypersensitivity reaction of with a time to maximum epinephrine plasma concentrations (Tmax) of less than 25 minutes.

60. The method of any one of claims 31-57, wherein the intranasal administration of the two doses plasma epinephrine concentrations in the human that are efficacious for the treatment of the type-1 hypersensitivity reaction of with a time to maximum epinephrine plasma concentrations (Tmax) of less than 15 minutes.

61. The method of any one of claims 31-60, wherein the pharmaceutical formulation is a nasal spray pharmaceutical formulation or a dry powder.

62. The method of any one of claims 31-61, wherein the pharmaceutical formulation is a nasal spray pharmaceutical formulation comprising between about 1 mg/mL and about 40 mg/mL of epinephrine, or a salt thereof, in a volume between about 25 μL and about 250 μL per dose.

63. The method of any one of claims 31-61, wherein each dose of the pharmaceutical formulation comprises between about 5 mg/mL and about 40 mg/mL of epinephrine, or a salt thereof.

64. The method of any one of claims 31-61, wherein each dose of the pharmaceutical formulation comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL, of epinephrine, or a salt thereof.

65. The method of any one of claims 31-61, wherein each dose of the pharmaceutical formulation comprises about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of epinephrine, or a salt thereof.

66. The method of any one of claims 31-61, wherein each dose of the pharmaceutical formulation comprises about 10 mg/mL of epinephrine, or a salt thereof, in a volume between about 50 μL and about 200 μL.

67. The method of any one of claims 31-61, wherein each dose of the pharmaceutical formulation comprises about 20 mg/mL of epinephrine, or a salt thereof, in a volume between about 50 μL and about 200 μL.

68. The method of any one of claims 31-67, wherein the pharmaceutical formulation further comprises one or more absorption enhancement agents as excipients.

69. The method of any one of claims 31-67, wherein the pharmaceutical formulation further comprises one or more absorption enhancement agents as excipients, and optionally one or more other agents as excipients selected from the group consisting of isotonicity agents, stabilizing agents, antioxidants, preservatives, and pH adjustment agents to adjust the pH to a final pH between about 3.0 and about 5.0; and water.

70. The method of any one of claims 31-67, wherein the pharmaceutical formulation further comprises one or more other agents as excipients selected from the group consisting of absorption enhancement agents, isotonicity agents, stabilizing agents, antioxidants, preservatives, and pH adjustment agents to adjust the pH to a final pH between about 3.0 and about 5.0; and water.

71. The method of any one of claims 31-67, wherein:
the absorption enhancement agents are selected from the group consisting of alkyl glycosides, fatty acids, bile salts, cyclodextrins, phospholipids, and alcohols;
the isotonicity agents are selected from the group consisting of dextrose, glycerin, mannitol, potassium chloride, and sodium chloride;
the stabilizing agent is ethylenediaminetetraacetic acid (EDTA) or disodium salt thereof;
the antioxidants are selected from the group consisting of alpha tocopherol, D-α-tocopherol polyethylene glycol 1000 succinate, ascorbic acid, isoascorbic acid, butylated hydroxyanisole, citric acid monohydrate, potassium metabisulfite, sodium bisulfite, sodium metabisulfite, and sodium sulfite;
the preservative is benzalkonium chloride; and
the pH adjustment agents are selected from the group consisting of adipic acid, ammonium chloride, citric acid, acetic acid, hydrochloric acid, lactic acid, phosphoric acid, propionic acid, sulfuric acid, tartaric acid, sodium hydroxide, sodium citrate, sodium bicarbonate, and sodium carbonate.

72. The method of any one of claims 68-71, wherein:
the absorption enhancement agents are selected from the group consisting of dodecyl maltoside, polysorbate 20, polysorbate 80, oleic acid, sodium lauryl sulfate, sodium glycocholate, sodium taurocholate, and sodium taurodihydrofusidate.

73. The method of any one of claims 68-71, wherein:
the absorption enhancement agents are selected from the group consisting of dodecyl maltoside, sodium glycocholate, sodium taurocholate, and sodium taurodihydrofusidate.

74. The method of any one of claims 68-71, wherein:
the absorption enhancement agent is dodecyl maltoside.

75. The method of any one of claims 31-74, wherein the salt of epinephrine is selected from the group consisting of epinephrine acetate, epinephrine hydrochloride, epinephrine tartrate, epinephrine bitartrate, epinephrine hydrogen tartrate, and epinephrine borate.

76. The method of any one of claims 69-75, wherein:
the one or more pH adjustment agents are selected from the group consisting of hydrochloric acid, sodium hydroxide, and combination thereof.

77. The method of any one of claims 31-61, wherein each intranasally delivered dose of the pharmaceutical formulation comprises about 10 mg/mL or about 20 mg/mL of epinephrine, or a salt thereof, in a volume between about 50 μL and about 200 μL; dodecyl maltoside, sodium chloride, optionally sodium metabisulfite, optionally ethylenediaminetetraacetic acid (EDTA) or disodium salt thereof, and optionally benzalkonium chloride; hydrochloric acid, sodium hydroxide, or both, in amounts to adjust the pH to a final pH between about 3.0 and about 5.0; and water.

78. The method of any one of claims 31-61, wherein each intranasally delivered dose of the pharmaceutical formulation comprises about 10 mg/mL to about 20 mg/mL of epinephrine, or a salt thereof, in a volume between about 50 μL and about 200 μL; dodecyl maltoside, sodium chloride; hydrochloric acid, sodium hydroxide, or both, in amounts to adjust the pH to a final pH between about 3.0 and about 5.0; and water.

79. The method of any one of claims 31-61, wherein each intranasally delivered dose of the pharmaceutical formulation comprises about 10 mg/mL or about 20 mg/mL of epinephrine, or a salt thereof, in a volume between about 50 μL and about 200 μL; dodecyl maltoside, sodium chloride, optionally sodium metabisulfite, optionally ethylenediaminetetraacetic acid (EDTA) or disodium salt thereof, and optionally benzalkonium chloride; hydrochloric acid, sodium hydroxide, or both, in amounts to adjust the pH to a final pH between about 3.0 and about 5.0; and water.

80. The method of any one of claims 31-79, wherein each intranasally delivered dose of the pharmaceutical formulation comprises about 20 mg/mL of epinephrine, or a salt thereof, in a volume of about 50 μL, about 75 μL, about 100 μL, about 125 μL, about 150 μL, about 175 μL, about 200 μL, or about 250 μL.

81. The method of any one of claims 31-61, wherein each intranasally delivered dose of the pharmaceutical formulation comprises about 10 mg/mL to about 20 mg/mL of epinephrine, or a salt thereof, in a volume between about 100 μL and about 140 μL; dodecyl maltoside, sodium chloride; hydrochloric acid, sodium hydroxide, or both, in amounts to adjust the pH to a final pH between about 3.0 and about 5.0; and water.

82. The method of any one of claims 31-61, wherein each intranasally delivered dose of the pharmaceutical formulation comprises about 10 mg/mL or about 20 mg/mL of epinephrine, or a salt thereof, in a volume between about 100 μL and about 140 μL; dodecyl maltoside, sodium chloride, optionally sodium metabisulfite, optionally ethylenediaminetetraacetic acid (EDTA) or disodium salt thereof, and optionally benzalkonium chloride; hydrochloric acid, sodium hydroxide, or both, in amounts to adjust the pH to a final pH between about 3.0 and about 5.0; and water.

83. The method of any one of claims 31-61, wherein each intranasally delivered dose of the pharmaceutical formulation comprises:
about 3 mg/mL, about 5 mg/mL, about 6.5 mg/mL, about 10 mg/mL, about 13 mg/mL,
about 15 mg/mL, or about 20 mg/mL of epinephrine in a volume between about 100 μL and about 140 μL;
about 2.5 mg/mL of dodecyl maltoside;
hydrochloric acid, sodium hydroxide, or combination thereof, to adjust the pH to a final pH between 3.0 and about 5.0; and
water.

84. The method of any one of claims 62-82, wherein each intranasally delivered dose of the pharmaceutical formulation further comprises:
optionally about 2.0 mg/mL of ethylenediaminetetraacetic acid (EDTA) or disodium salt thereof;
optionally about 0.4 mg/mL of benzalkonium chloride as an excipient;
about 8.23 mg/mL of sodium chloride as an excipient;
optionally about 0.05 mg/mL of sodium metabisulfite as an excipient.

85. The method of any one of claims 62-84, wherein:
each dose of the nasal spray is intranasally administered with a nasal spray device; and
one actuation of the nasal spray device delivers about 100 μL of the pharmaceutical formulation.

86. The method of any one of claims 62-84, wherein:
each dose of the nasal spray is intranasally administered with a single-dose nasal spray device; and
one actuation of the nasal spray device delivers about 100 μL of the pharmaceutical formulation.

87. The method of any one of claims 31-61, wherein the pharmaceutical formulation is a dry powder in the form of a solid, amorphous, mono-particulate powder comprising a mixture of:
(a) epinephrine, or a pharmaceutically acceptable salt thereof; and
(b) a pharmaceutically-acceptable carrier material.

88. The method of claim 87, wherein:
the carrier material comprises a maltodextrin with a dextrose equivalent (DE) that is above 15;
wherein the powder comprises particles of a size whereby, upon intranasal administration of said powder, said powder is delivered to nasal mucosa.

89. The method of any one of claims 57-88, wherein the carrier material further comprises a disaccharide, selected from the group consisting of maltitol, trehalose, sucralose, sucrose, isomalt, maltose and lactose.

90. The method of any one of claims 87-89, wherein the disaccharide comprises lactose and/or trehalose.

91. The method of any one of claims 87-90, wherein the carrier material comprises a combination of trehalose and maltodextrin 19DE.

92. The method of any one of claims 87-91, wherein the ratio of disaccharide:maltodextrin by weight, based on the total weight of the composition, is in the range of about 10:1 to about 1:20.

93. The method of any one of claims 87-91, wherein the ratio of disaccharide:maltodextrin by weight, based on the total weight of the composition, is in the range of about 2:1 to about 1:8.

94. The method of any one of claims 87-93, wherein the composition further comprises a sucrose ester.

95. The method of any one of claims 87-94, wherein the sucrose ester comprises sucrose monolaurate.

96. The method of any one of claims 87-95, wherein each dose comprises between about 0.5 mg and about 3 mg of epinephrine.

97. The method of any one of claims 87-96, wherein the particles of the amorphous powder have a size distribution with a D10 value above about 10 μm and a D90 value below about 500 μm or below about 100 μm.

98. The method of any one of claims 87-97, wherein the particles of said powder have a D90 below about 100 μm.

99. The method of claim 87, wherein the pharmaceutically-acceptable carrier material is lactose.

100. The method of claim 99, wherein each dose comprises between about 0.5 mg and about 4 mg of epinephrine.

101. The method of claim 99, wherein each dose comprises about 1.6 mg or about 3.2 mg.
